(19) United States
(12) Patent Application Publication
(10) Pub. No.: US 2008/0214606 A1
(43) Pub. Date: Sep. 4, 2008

(54) Methods for the Identification and Use of Compounds Suitable for the Treatment of Drug Resistant Cancer Cells

(75) Inventors: Gergely Szakacs, Budapest (HU); Jean-Phillipe Annereau, Toulouse (FR); Samir Lababidi, Rockville, MD (US); Michael M. Gottesman, Bethesda, MA (US); John Weinstein, Chevy Chase, MD (US)

Correspondence Address: KLARQUIST SPARKMAN, LLP, 121 S.W. SALMON STREET, SUITE #1600, PORTLAND, OR, 97204-2988, US

(73) Assignee: The Government of the United States of America as represented by The Secretary of the Dept. of ....., Rockville, MD (US)

(21) Appl. No.: 11/629,233

(22) PCT Filed: Jun. 16, 2005
(86) PCT No.: PCT/US05/21253

§ 371 Date: Dec. 7, 2006

Related U.S. Application Data
(60) Provisional application No. 60/580,397, filed on Jun. 18, 2004.
(60) Provisional application No. 60/602,640, filed on Aug. 19, 2004.

(51) Int. Cl. A61K 31/404; A61K 31/47; C12Q 1/68; A61P 35/00

(52) U.S. Cl. 514/313; 514/418; 435/6

(57) ABSTRACT

The present invention relates to novel methods for the identification of compounds useful for the treatment of drug resistance, and to novel treatment methods using the identified compounds.

### CROSS-REFERENCE TO RELATED APPLICATIONS

This application claims the benefit of U.S. Patent Application Ser. Nos. 60/602,640 (filed on Aug. 19, 2004) and 60/580,397 (filed on Jun. 18, 2004), both of which applications are herein incorporated by reference in their entirety.

### FIELD OF THE INVENTION

The present invention relates to novel methods for the identification of compounds useful for the treatment of drug resistant cells, and to novel treatment methods using the identified compounds.

### BACKGROUND OF THE INVENTION

Drug resistance is one of the primary causes of treatment failure in cancer therapy. ATP-binding cassette (ABC) transporters are a family of transporter proteins that contribute to drug resistance via ATP-dependent drug efflux pumps (Gottesman et al., 2002, Multidrug resistance in cancer: role of ATP-dependent transporters, Nat. Rev. Cancer 2(1):48-58). P-glycoprotein (P-gp), encoded by the ABCB1 gene (also referred to as the MDR1 gene), is an ABC transporter that normally functions to excrete xenobiotics from cells. Expression of the ABCB1 protein also confers resistance to certain chemotherapeutic agents including vinca alkaloids, anthracyclines, epipodophyllotoxines, actinomycin D and taxanes. P-gp is over-expressed at diagnosis in certain chemotherapy resistant tumors and is upregulated after disease progression following chemotherapy in other malignancies.

Other ABC transporter proteins known to mediate clinical drug resistance include the multidrug-resistance-associated-protein 1 (MRP1, or ABCC1) and ABCG2, also known as MXR (mitoxantrone-resistance gene), BCRP (breast cancer resistance protein) and ABC-P (ABC transporter in placenta).

One approach to overcome drug resistance in cancer therapy includes the development of inhibitors of ABC transporters to be used in conjunction with chemotherapy. Although a considerable amount of resources have been expended in the identification and development of inhibitors of ABCB1 (MDR1) for use in cancer therapy, this approach has not proven to be clinically successful to date.

Anti-cancer therapy that mitigates the development of drug resistance is an unmet public health need. The present invention is directed to address this need.

### SUMMARY OF THE INVENTION

In one aspect, the invention relates to a method of inhibiting the growth of neoplastic cells in a subject comprising administering to the subject an antiproliferative agent, wherein the antiproliferative effect of the agent is potentiated by the ABCB1 transporter.

Particularly, the invention relates to a method of inhibiting the growth of a cancer in a subject comprising administering to the subject an antiproliferative agent, wherein the antiproliferative effect of the agent is potentiated by the ABCB1 transporter, and wherein the cancer exhibits a multidrug resistance phenotype.

In another aspect, the invention relates to a method of inhibiting the growth of a cancer in a subject comprising administering to the subject an antiproliferative agent, wherein the antiproliferative effect of the agent is potentiated by the ABCB1 transporter, and wherein the subject has previously been treated with at least one anti-cancer therapeutic agent that is an ABCB1 substrate.

In another aspect, the invention relates to a method of inhibiting the development of multidrug resistance in a cancer in a subject comprising administering to the subject an antiproliferative agent, wherein the antiproliferative effect of the antiproliferative agent is potentiated by the ABCB1 transporter.

In another aspect, the invention relates to a method of identifying therapeutic compounds having a therapeutic activity that is potentiated by the expression of an ABC gene comprising the steps of: (a) determining the expression level of at least one ABC gene in a panel of cell lines; (b) determining the level of therapeutic activity of at least one test compound on the panel of cell lines; and (c) correlating the level of therapeutic activity with the expression level of the ABC gene, wherein a positive correlation between the level of therapeutic activity and the expression level of the ABC gene identifies the test compound as having an activity that is potentiated by the expression of the ABC gene.

In another aspect, the invention relates to a method of identifying therapeutic compounds as substrates for ABC transporters comprising the steps of: (a) determining the expression level of at least one ABC gene in a panel of cell lines; (b) determining the level of therapeutic activity of at least one test compound on the panel of cell lines; (c) comparing the level of therapeutic activity with the expression level of the ABC gene, wherein a negative correlation between the level of therapeutic activity and the expression level of the ABC gene identifies the test compound as a substrate of the ABC transporter encoded by the ABC gene.

In another aspect, the invention relates to a method of inhibiting the growth of neoplastic cells in a subject comprising administering to the subject an antiproliferative agent, wherein the antiproliferative effect of the agent is potentiated by the ABCB1 transporter, wherein the antiproliferative agent is a compound of Structure Y or Structure Z:

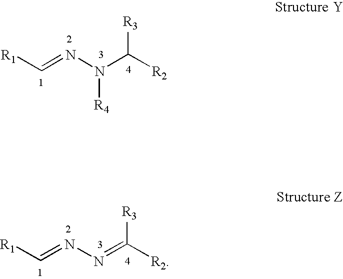

wherein R₁ may comprise one or two substituents on the carbon atom in position 1;

wherein each of R₁ are independently selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;

wherein when R₁ comprises two substituents on the carbon atom in position 1, the two substituents may cyclize to form a ring structure;

wherein each of R₁ may independently cyclize to form a ring structure;

wherein R₂ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;

wherein R₂ may cyclize to form a ring structure;

wherein R₃ comprises 0 or 1 substituents on the carbon atom at position 4;

wherein R₃ may be double bonded or single bonded to the carbon atom at position 4 of Structure Y or single bonded to the carbon atom at position 4 of Structure Z;

wherein R₃ is selected from the group consisting of a heteroatom, hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;

wherein R₃ may cyclize to form a ring structure;

wherein R₄ comprises 0 or 1 substituents on the nitrogen atom at position 3 of Structure Y or Structure Z;

wherein R₄ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;

wherein R₄ may cyclize to form a ring structure.

### BRIEF DESCRIPTION OF THE FIGURES

FIG. 1 is a clustered image map of ABC transporter gene expression in the NCI-60 human cancer cell panel. Gene expression is assessed by real-time RT-PCR. Medium gray and light gray indicate high and low expression, respectively. Hierarchical clustering on each axis is done using the average-linkage algorithm with 1−r as the distance metric, where r is the Pearson's correlation coefficient, after subtracting row and column means. The inset highlights ABC transporters characteristically expressed in melanoma cells. The data presented graphically in FIG. 1 is presented numerically in Table 3.

FIG. 2 depicts the relationship between drug sensitivity and ABCB1 expression in the NCI-60 for a set of 118 drugs having putatively known mechanisms of action. Dotted/dashed bars indicate known ABCB1 substrates; dashed bars indicate compounds shown in previous studies not to be substrates of ABCB1; solid bars indicate compounds for which data were not available from the literature. Commonly used names for representative agents of the classes are shown in the boxes.

FIG. 3 shows further experimental results demonstrating the identification of novel ABCB1 substrates using the NCI-60 correlation analysis. Panel A is a scatter plot showing the correlation (r) of ABCB1 expression with sensitivity of the 60 cells to NSC 363997 (r=−0.59; 99.99% two-tailed bootstrap confidence interval −0.8488 to −0.1130). Panel B shows MIT assay dose response curves for treatment of KB-3-1 parental cancer cells and the selected resistant variant KB-V-1 with increasing concentrations of NSC 363997. The dashed lines indicate the same experiment performed in the presence of 2 μM of the ABCB1 inhibitor, PSC 833 (for KB-3-1, the solid and dashed lines overlap). Values are means ±SE. for representative experiments performed in triplicate. Panel C shows a summary of further, analogous cytotoxicity assays performed using five other compounds. Concentrations resulting in 50% cell death (IC₅₀) in the absence and presence (values in parentheses) of 2 μM PSC 833 are shown in μmoles/liter. The effect of PSC 833 on IC₅₀ values in KB-V1 cells is expressed as a dose modifying factor, DMF=[IC₅₀/IC₅₀+(IC₅₀)PSC833)], where (IC₅₀)PSC833 is the value obtained in the presence of the inhibitor. Panel D shows an analysis of the accumulation of the intrinsically fluorescent compound NSC 634791 in MDR1-overexpressing KB-V1 cells. Cells are incubated with 1.74 μM NSC 634791 for 10 min at 37° C. in the presence (peak on the right) or absence (peak on the left) of 2 μM PSC 833.

FIG. 4 shows experimental results demonstrating the identification of a new substrate for ABCC2 (MRP2) with the NCI-60 correlation analysis. Panel A is a scatter plot showing the correlation (r) of ABCC2 expression with sensitivity of the 60 cells to NSC 641281 (r=−0.46; 99.99% two-tailed bootstrap confidence interval −0.7987 to −0.0440). Panel B shows dose response curves for treatment of sham-transfected and ABCC2-transfected MDCCKII dog kidney cells with NSC 641281. The ABCC2-expressing cells showed no signs of toxicity even at maximal concentrations. Panel C shows the structure of NSC 641281.

FIG. 5 shows experimental results demonstrating the identification of a new substrate for ABCC11 (MRP8) with the NCI-60 correlation analysis. Panel A is a scatter plot showing the correlation (r) of ABCC11 expression with sensitivity of the 60 cells to NSC 671136 (r=−0.4; 99.99% two-tailed bootstrap confidence interval −0.6726 to −0.0141). Removal of the single, high-expressing cell line (T47D) from the analysis does not significantly reduce the observed correlation (r=−0.38; 99.99% confidence interval −0.7233 to −0.03915). Panel B shows dose response curves for treatment of sham-transfected and ABCC11-transfected LLCPK1 non-small cell lung cancer cells with NSC 671136. Values are means ±S.E. of triplicate MTT assays. Panel C shows the structure of NSC 671136.

FIG. 6 shows experimental results demonstrating the identification via the NCI-60 correlation analysis of antiproliferative agents that are potentiated, rather than inhibited, by the expression of ABCB1. Panel A is a scatter plot showing positive correlation (r=+0.54; 95% confidence interval 0.259 to 0.713) of ABCB1 expression with sensitivity of the 60 cell lines to NSC 73306. Panel B shows dose-response curves indicating that, in an MTT assay, selected resistant KB-V-1 cells are approximately four-fold more sensitive to NSC 73306 than are parental KB-3-1 cells. Dashed lines indicate the corresponding results in the presence of 2 μM PSC 833, which completely abolished the heightened sensitivity of KB-V-1. Panel C shows dose-response curves of KB Hela cells expressing ABCB1(MDR1) under tetracycline control exposed to NSC 73306. Cells are grown in the absence (ABCB1(MDR1)-On) or presence (ABCB1(MDR1)-Off) of 2 μg/ml tetracycline for at least seven days before starting the MTT assay. Cell surface expression and function of ABCB1 (MDR1) are verified prior to the assay by staining with anti-MDR1 monoclonal antibody (MRK-16) and by a performing a functional assay based on MDR1-controlled accumulation of the fluorescent dye Calcein (Homolya et al., 1996, Br. J. Cancer 73:849-855). The MTT assay shows an approximately two-fold higher sensitivity to NSC 73306 with upregulation of ABCB1(MDR1). Values are means ±S.E. of triplicate measurements.

### DESCRIPTION OF THE PREFERRED EMBODIMENTS

In one aspect, the invention relates to the recognition that certain antiproliferative compounds have an antiproliferative activity that is potentiated (i.e., enhanced, greater, improved or rendered more potent) rather than inhibited by expression of ABCB1 (MDR1) (see, Szakács, G. et al. (2004) “Predicting Drug Sensitivity and Resistance: Profiling ABC Transporter Genes in Cancer Cells,” Cancer Cell, 6:129-137 (and Supplementary Files thereof, http://discover.nci.nih. gov/abc/2004_cancercell_abstractjsp#supplement), herein incorporated by reference). Thus, the invention relates to methods of treating neoplastic disease in a subject in need of such treatment through the administration of such compounds. The methods and compositions of the present invention may be used in any species affected by neoplastic disease, including humans and non-human animals (e.g., non-human mammals and birds).

An “ABCB1 potentiated compound”, as used herein, refers to any compound whose antiproliferative effect on a cell is potentiated rather than inhibited by the ABCB1 protein. With the teaching of this invention, one of ordinary skill in the art could readily determine whether any particular compound is an ABCB1 potentiated compound. For example, assay methods using a cell line that has been genetically engineered to express or over-express the ABCB1 transporter, as described in the examples herein, may be employed. Preferred ABCB1 potentiated compounds of the invention are compounds having an antiproliferative effect that is at least 1.5 fold, 2-fold, 3-fold, 4-fold 5-fold, or 6-fold greater in genetically engineered cells (i.e. genetically engineered to express or over express the ABCB1 transporter) than in control cells.

The ABCB1 potentiated compounds of the invention are useful in the treatment of a variety of cancers and other proliferative diseases and neoplastic conditions. For example, and without limitation, treatment of the following cancers is contemplated: carcinoma, including that of the bladder, breast, colon, kidney, liver, lung, ovary, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, and thyroid follicular cancer.

In a preferred embodiment of the invention, the ABCB1 potentiating compounds will be useful for the treatment of cancers exhibiting a multiple drug resistance (“MDR”) phenotype or having a substantial probability for development of an MDR phenotype. As used herein, an “MDR phenotype” refers to a cancer showing resistance to cancer therapeutic agents that are substrates of the ABCB1 transporter. Such therapeutic agents include, by way of example and not by limitation, anthracyclines (e.g. daunorubicin (Cerubidine), doxorubicin (Adriamycin, Rubex), epirubicin (Ellence, Pharmorubicin), idarubicin (Idamycin)), vinca alkaloids (e.g. vinblastine, vincristine, vindesine, vinorelbine), taxanes (e.g. paclitaxel, docetaxel), and epipodophyllotoxins (e.g. etoposide).

For any particular cancer, the presence or absence of an MDR phenotype can be readily determined in a number of ways using techniques that are well known in the art. For example, treatment of a subject with a cancer therapeutic agent that is known to be a substrate of ABCB1 (e.g., an anthracycline, a taxane, a vinca alkaloid, or an epipodophyllotoxin) and the subsequent development of cancer that is resistant to the therapeutic agent would indicate the presence of an MDR phenotype. Alternatively, a high level of expression or functionality of the ABCB1 gene or protein in a cancer would be indicative of an MDR phenotype. The level of expression or functionality of the ABCB1 gene or protein may be assessed in vitro, using harvested cells. For example, calcein-AM is useful for the qualitative functional analysis of the presence of multi-drug resistance in cells (Hollo, 1994, Biochim. Biophys. Acta 1191:384; U.S. Pat. Nos. 6,277,655 and 5,872,014). Additionally, the level of expression or functionality of the ABCB1 gene or protein may be assessed in vivo using, for example, the techniques of single photon emission tomography (SPECT) and positron emission tomography (PEI), in combination with a detectable (e.g. radiolabeled) ABCB1 substrate (Hendrike and Vaalburg, 2002, Methods 27(3):228-233; Hendrikse et al., 1999, Cancer Res. 59(10):2411-2416) or by using a bioluminescence approach Pichler et al., 2004, Proc. Natl. Acad. Sci. USA 101(6)1702-1707. Methods of assaying the reversal of the multidrug resistance phenotype through the use of specific ABCB1 transporter inhibitors, such as for example, PSC 833, may also be used to establish the existence of an MDR phenotype.

Cancers exhibiting an MDR phenotype may be cancers that present with an MDR phenotype at diagnosis or cancers that do not have an MDR phenotype at diagnosis, but which develop such a phenotype during the course of chemotherapeutic treatment. Cancers that may present with an MDR phenotype at diagnosis include, for example, colon carcinoma, renal carcinoma, hepatoma, adrenocortical carcinoma, and pancreatic carcinoma. Several types of cancer are known to develop an MDR phenotype through upregulation of the ABCB1 gene, concomitant overexpression of P-glycoprotein (P-gp), during the course of chemotherapeutic treatment including the following: a wide variety of solid tumors, particularly breast cancer, ovarian cancer, sarcoma, and small cell lung cancer (Kaye, 1998, Curr. Opin Oncol., 10 Suppl 1:S15-19) and certain leukemias (acute myeloid leukemia, chronic myeloid leukemia, acute lymphoblastic leukemia) and lymphomas (non-Hodgkins lymphoma, B cell lymphoma, T cell lymphoma) (Hart et al, 1993, Leuk Lymphoma 11: 239-248; Yamaguchi et al., 1995, Cancer 76: 2351-2356). Thus, identification of the cancer type can be used to identify a cancer that has a substantial probability of developing an MDR phenotype.

ABCB1 potentiated compounds may be identified using the teaching of this invention and the techniques described herein. Preferred ABCB1 potentiated compounds are those described in Tables 7, 8, and 9, and derivatives of these compounds. It has been demonstrated as part of the invention described herein that these compounds have an anti-proliferation effect that is potentiated by ABCB1 transporters. It is within the scope of one of skill in the art to modify these compounds to achieve enhanced antiproliferation effect, or to achieve other desirable properties such as enhanced solubility or desirable in vivo pharmacokinetic properties and toxicity profiles.

In a preferred embodiment, the invention relates to methods of treating cancer in a subject with an ABCB1 potentiated agent, wherein the subject has been previously treated for the same cancer with a chemotherapeutic agent that is a substrate of the ABCB1 transporter. For example, the chemotherapeutic agent may be selected from the group consisting of a taxane, an anthracycline, a vinca alkaloid, or an epipodophyllotoxin.

In another preferred embodiment, the invention relates to methods of inhibiting the development of a multidrug resistance phenotype in a cancer in a subject comprising administering an ABCB1 potentiated agent to the subject. As used herein, inhibiting the development of a multidrug resistant phenotype refers to both the inhibition of the initial onset of the phenotype or the inhibition of any further development of the multidrug phenotype. It is contemplated as part of the invention that the ABCB1 potentiated agent may be administered simultaneously with a chemotherapeutic agent that is a substrate of the ABCB1 transporter. It is understood as an aspect of the invention that such simultaneous administration refers to administration within the same general time period rather than at the same exact moment in time. Thus treatment with the ABCB1 potentiated compound and the chemotherapeutic agent may be on the same day or on different days, or in the same week or in different weeks. It is within the skill of the ordinary artisan to optimize a treatment schedule to maintain the therapeutic efficacy of the chemotherapeutic agent by administration of the ABCB1 potentiated compound to inhibit the development of drug resistance. MDR1-potentiated compounds may be used to prevent the emergence of drug resistance clones. Cells expressing high levels of endogenous MDR1 (as a result of selection, or high initial expression), as well as cells engineered to express high levels of MDR1, lose their MDR phenotype upon incubation in MDR1-potentiated compounds. The loss of the MDR phenotype is due to the loss of MDR1 expression. The loss of MDR1 expression and the concomitant loss of the MDR phenotype may be a result of selection (i.e. the selective loss of MDR1-positive cells) or induction (i.e. the downregulation of MDR1 expression in cells).

Pretreatment of MDR1 positive cells with NSC73306 results in almost complete elimination of drug resistance to MDR1 substrates. In contrast, drug sensitivity is unchanged for non-MDR1 substrates (such as cisplatin and methotrexate), suggesting that “resensitization” occurs through loss of MDR1, not by other non-specific mechanisms such as altered cell growth kinetics or metabolism. Interestingly, even low doses (around IC50) of MDR1-potentiated compounds (such as 73306) bring about this effect, suggesting that treatment protocols could contain doses below the cytotoxic concentration. In summary, we suggest that MDR1-potentiated compounds may be used prior to treatment with cytotoxic chemotherapy, to prevent the upregulation of MDR1.

MDR1 potentiated compounds of the invention include: NSC 292408; NSC 10580; NSC 716768; NSC 73306; NSC 713048; NSC 168468; NSC 657441; NSC 302325; and NSC 657456. Additionally, structural analogs of these compounds are also MDR1-potentiated. Exemplary analogs include analogs of NSC 168468 such as NSC 168466; NSC 687208; NSC 687209; NSC 687210; NSC 168467; NSC 1604; etc.; analogs of NSC 292408 such as NSC 615541, 1-10 phenanthroline, etc.; and analogs of NSC 713048 such as NSC 696920; NSC 704347; etc. The identification of the activity of such structural analogs is relevant because analogs that retain MDR1-potentiated activity can be used to reveal the pharmacophore. Note that structural analogs were identified by (1) correlating expression with sensitivity, and (2) identifying structural analogs of promising compounds. Thus, the toxicity profiles of structural analogs are not necessarily highly correlated to MDR1 expression. The structures of such compounds are indicated below.

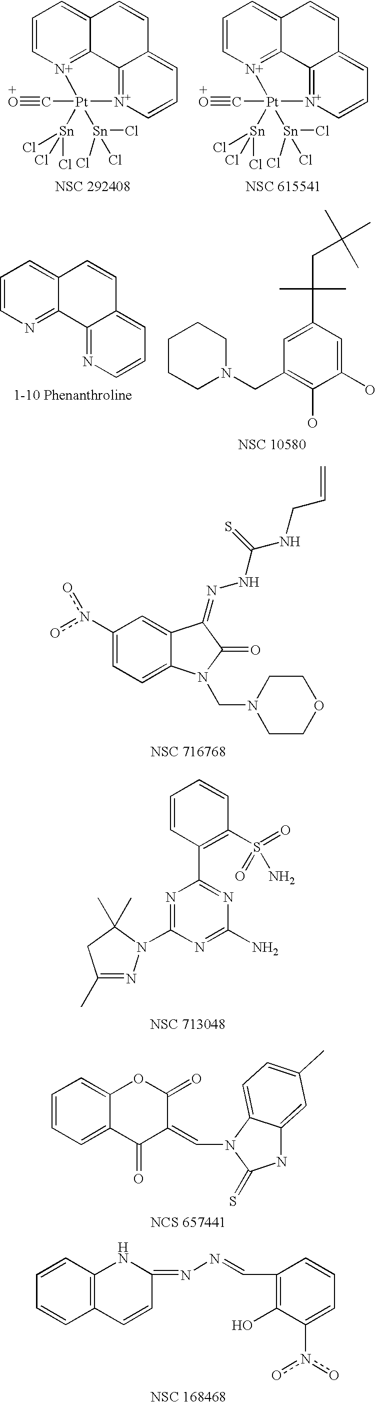

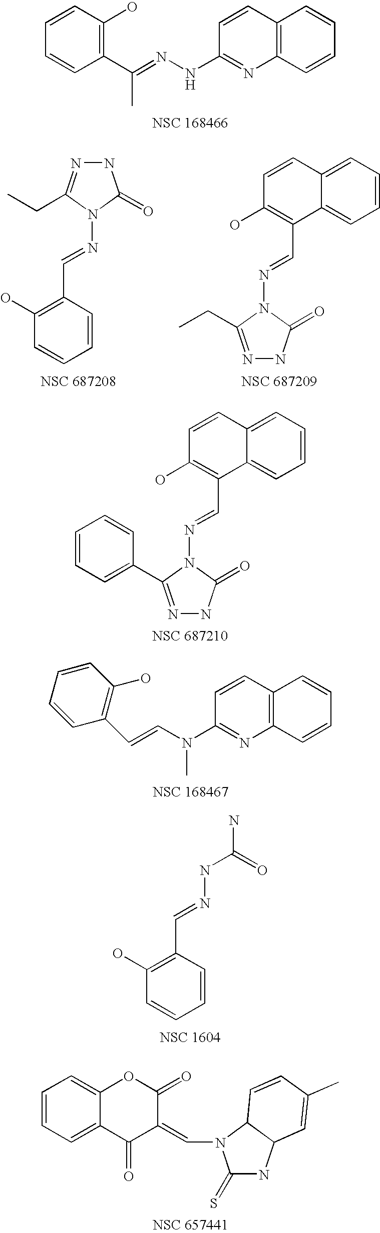

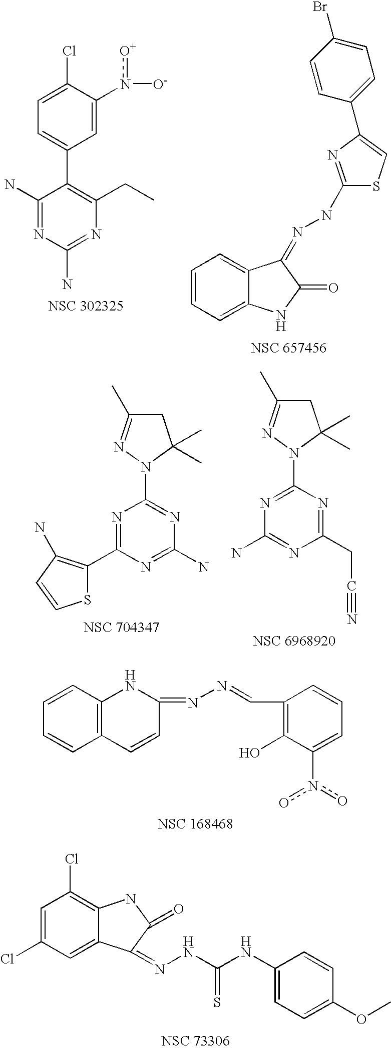

In a preferred embodiment, ABCB1 potentiated compounds of the invention have the following Structure X:

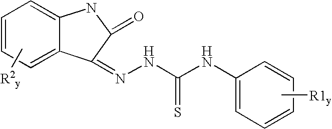

Wherein R¹ and R² are each independently selected from the group consisting of a halogen atom, a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
Wherein y is 0 to 3 (independently for each of R¹ and R²), preferably 0 to 2.

### Wherein X is O or S.

In preferred embodiments, y is 0 to 2, X is S, and R¹ and R² are each independently selected from the group consisting of a halogen atom, NO₂, methyl, and a heterogeneous group having 2-3 member atoms in the chain.

Preferred ABCB1 potentiated compounds of the invention include, for example, the compounds listed below and derivatives of these compounds:

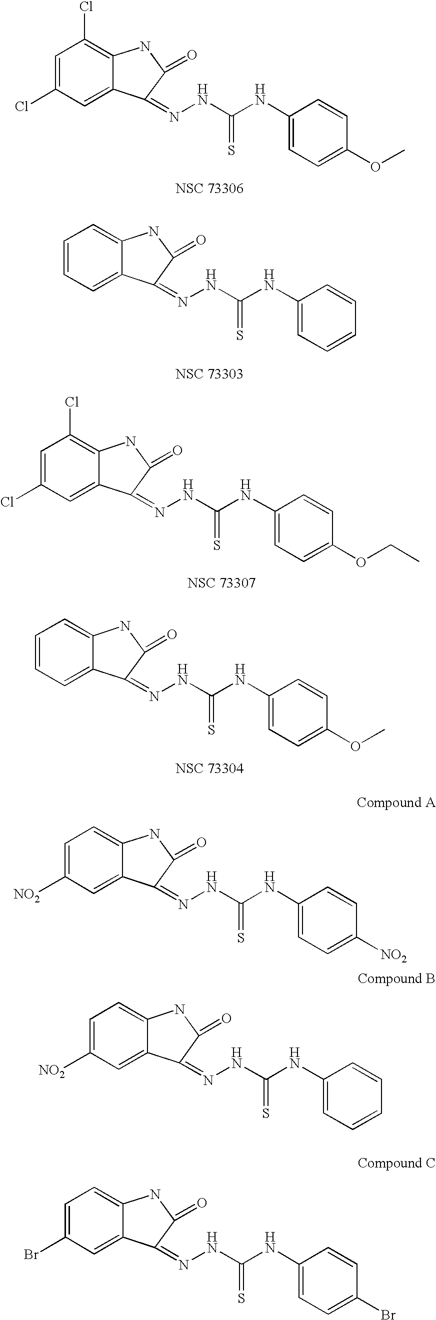

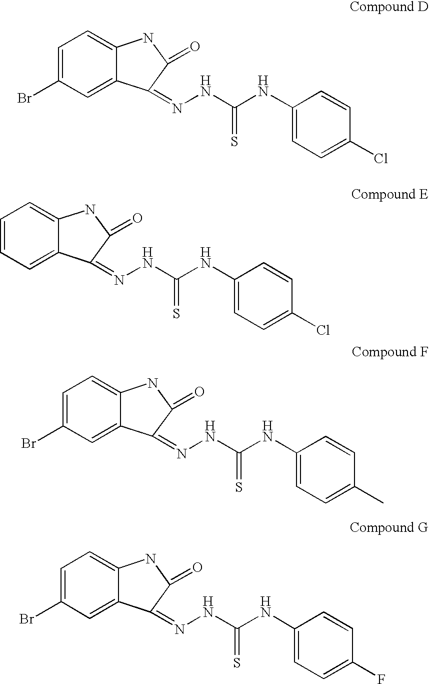

As used herein, “aromatic group” means an aromatic group having a monocyclic or polycyclic ring structure. Monocyclic aromatic groups contain 4 to 10 carbon atoms, preferably 4 to 7 carbon atoms, and more preferably 4 to 6 carbon atoms in the ring. Preferred polycyclic ring structures have two or three rings. Polycyclic structures having two rings typically have 8 to 12 carbon atoms, preferably 8 to 10 carbon atoms in the rings. Polycyclic aromatic groups include groups wherein at least one, but not all, of the rings are aromatic.

As used herein, “carbocyclic group” means a saturated or unsaturated carbocyclic hydrocarbon ring. Carbocyclic groups are not aromatic. Carbocyclic groups are monocyclic or polycyclic. Polycyclic carbocyclic groups can be fused, spiro, or bridged ring systems. Monocyclic carbocyclic groups contain 4 to 10 carbon atoms, preferably 4 to 7 carbon atoms, and more preferably 5 to 6 carbon atoms in the ring. Bicyclic carbocyclic groups contain 8 to 12 carbon atoms, preferably 9 to 10 carbon atoms in the rings.

As used herein, “heteroaromatic group” means an aromatic group containing carbon and 1 to 4 heteroatoms in the ring. Monocyclic heteroaromatic groups contain 4 to 10 member atoms, preferably 4 to 7 member atoms, and more preferably 4 to 6 member atoms in the ring. Preferred polycyclic ring structures have two or three rings. Polycyclic structures having two rings typically have 8 to 12 member atoms, preferably 8 to 10 member atoms in the rings. Polycyclic heteroaromatic groups include groups wherein at least one, but not all, of the rings are heteroaromatic.

As used herein, “heteroatom” means an atom other than carbon, e.g., in the ring of a heterocyclic group or the chain of a heterogeneous group. Preferably, heteroatoms are selected from the group consisting of sulfur, phosphorous, nitrogen and oxygen atoms. Groups containing more than one heteroatom may contain different heteroatoms.

As used herein, “heterocyclic group” means a saturated or unsaturated ring structure containing carbon atoms and 1 or more heteroatoms in the ring. Heterocyclic groups are not aromatic. Heterocyclic groups are monocyclic or polycyclic. Polycyclic heteroaromatic groups can be fused, spiro, or bridged ring systems. Monocyclic heterocyclic groups contain 4 to 10 member atoms (i.e., including both carbon atoms and at least 1 heteroatom), preferably 4 to 7, and more preferably 5 to 6 in the ring. Bicyclic heterocyclic groups contain 8 to 18 member atoms, preferably 9 or 10 in the rings.

As used herein, “heterogeneous group” means a saturated or unsaturated chain of non-hydrogen member atoms comprising carbon atoms and at least one heteroatom. Heterogeneous groups typically have 1 to 25 member atoms. Preferably, the chain contains 1 to 12 member atoms, more preferably 1 to 10, and most preferably 1 to 6. The chain may be linear or branched. Preferred branched heterogeneous groups have one or two branches, preferably one branch. Preferred heterogeneous groups are saturated. Unsaturated heterogeneous groups have one or more double bonds, one or more triple bonds, or both. Preferred unsaturated heterogeneous groups have one or two double bonds or one triple bond. More preferably, the unsaturated heterogeneous group has one double bond.

As used herein, “hydrocarbon group” means a chain of 1 to 25 carbon atoms, preferably 1 to 12 carbon atoms, more preferably 1 to 10 carbon atoms, and most preferably 1 to 8 carbon atoms. Hydrocarbon groups may have a linear or branched chain structure. Preferred hydrocarbon groups have one or two branches, preferably 1 branch. Preferred hydrocarbon groups are saturated. Unsaturated hydrocarbon groups have one or more double bonds, one or more triple bonds, or combinations thereof. Preferred unsaturated hydrocarbon groups have one or two double bonds or one triple bond; more preferred unsaturated hydrocarbon groups have one double bond.

As used herein, “substituted aromatic group” means an aromatic group wherein 1 or more of the hydrogen atoms bonded to carbon atoms in the ring have been replaced with other substituents. Preferred substituents include hydrocarbon groups such as methyl groups and heterogeneous groups including alkoxy groups such as methoxy groups. The substituents may be substituted at the ortho, meta, or para position on the ring, or any combination thereof.

As used herein, “substituted carbocyclic group” means a carbocyclic group wherein 1 or more hydrogen atoms bonded to carbon atoms in the ring have been replaced with other substituents. Preferred substituents include hydrocarbon groups such as alkyl groups (e.g., methyl groups) and heterogeneous groups such as alkoxy groups (e.g., methoxy groups).

As used herein, “substituted heteroaromatic group” means a heteroaromatic group wherein 1 or more hydrogen atoms bonded to carbon atoms in the ring have been replaced with other substituents. Preferred substituents include monovalent hydrocarbon groups including alkyl groups such as methyl groups and monovalent heterogeneous groups including alkoxy groups such as methoxy groups.

As used herein, “substituted heterocyclic group” means a heterocyclic group wherein 1 or more hydrogen atoms bonded to carbon atoms in the ring have been replaced with other substituents. Preferred substituents include monovalent hydrocarbon groups including alkyl groups such as methyl groups and monovalent heterogeneous groups including alkoxy groups such as methoxy groups. Substituted heterocyclic groups are not aromatic.

As used herein, “substituted heterogeneous group” means a heterogeneous group, wherein 1 or more of the hydrogen atoms bonded to carbon atoms in the chain have been replaced with other substituents. Preferred substituents include monovalent hydrocarbon groups including alkyl groups such as methyl groups and monovalent heterogeneous groups including alkoxy groups such as methoxy groups.

As used herein, “substituted hydrocarbon group” means a hydrocarbon group wherein 1 or more of the hydrogen atoms bonded to carbon atoms in the chain have been replaced with other substituents. Preferred substituents include monovalent aromatic groups, monovalent substituted aromatic groups, monovalent hydrocarbon groups including alkyl groups such as methyl groups, monovalent substituted hydrocarbon groups such as benzyl, and monovalent heterogeneous groups including alkoxy groups such as methoxy groups.

Additional preferred ABCB1 potentiated compounds of the invention are the compounds listed below and derivatives of those compounds.

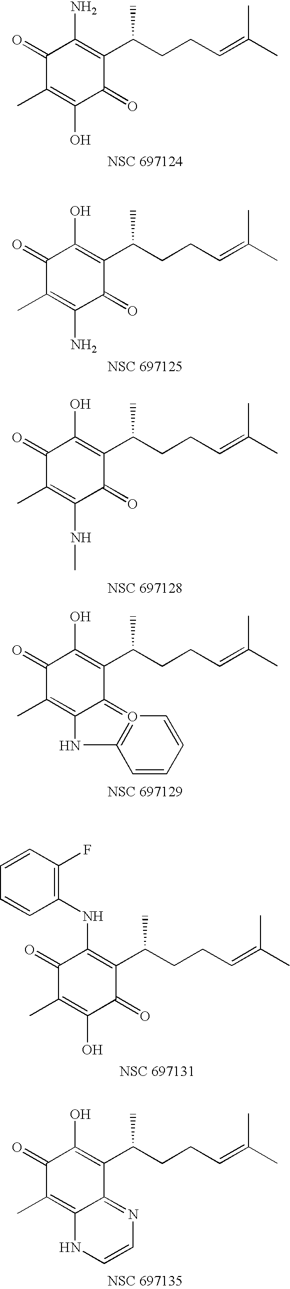

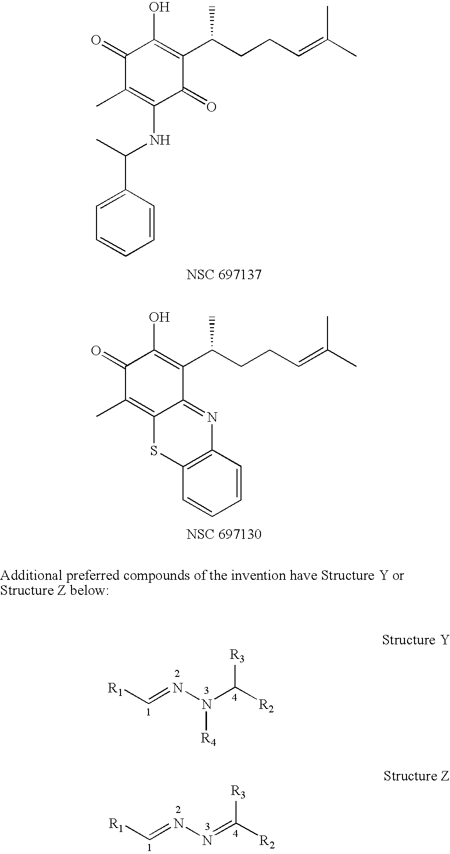

wherein R₁ may comprise one or two substituents on the carbon atom in position 1;
wherein each of R₁ are independently selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein when R₁ comprises two substituents on the carbon atom in position 1, the two substituents may cyclize to form a ring structure;
wherein each of R₁ may independently cyclize to form a ring structure;
wherein R₂ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein R₂ may cyclize to form a ring structure;
wherein R₃ comprises 0 or 1 substituents on the carbon atom at position 4;
wherein R₃ may be double bonded or single bonded to the carbon atom at position 4 of Structure Y or single bonded to the carbon atom at position 4 of Structure Z;
wherein R₃ is selected from the group consisting of a heteroatom, hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein R₃ may cyclize to form a ring structure;
wherein R₄ comprises 0 or 1 substituents on the nitrogen atom at position 3 of Structure Y or Structure Z;
wherein R₄ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein R₄ may cyclize to form a ring structure.

In preferred embodiments R₂ is —N—R₅,

wherein R₂-may be single bonded or double bonded to the carbon atom at position of 4 of Structure Y or single bonded to the carbon atom at position 4 of Structure Z;
wherein R₅ comprises one or two substituents on the nitrogen atom;
wherein when R₅ comprises one substituent on the nitrogen atom and R₂ is single bonded to the carbon atom at position 4 of Structure Y or Z, R₅ may be double bonded to the nitrogen atom;
wherein each of R₅ may independently cyclize to form a ring structure;
wherein each of R₅ is independently selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group.

Examples of compounds having the structure of Structure Y or Structure Z above are listed below:

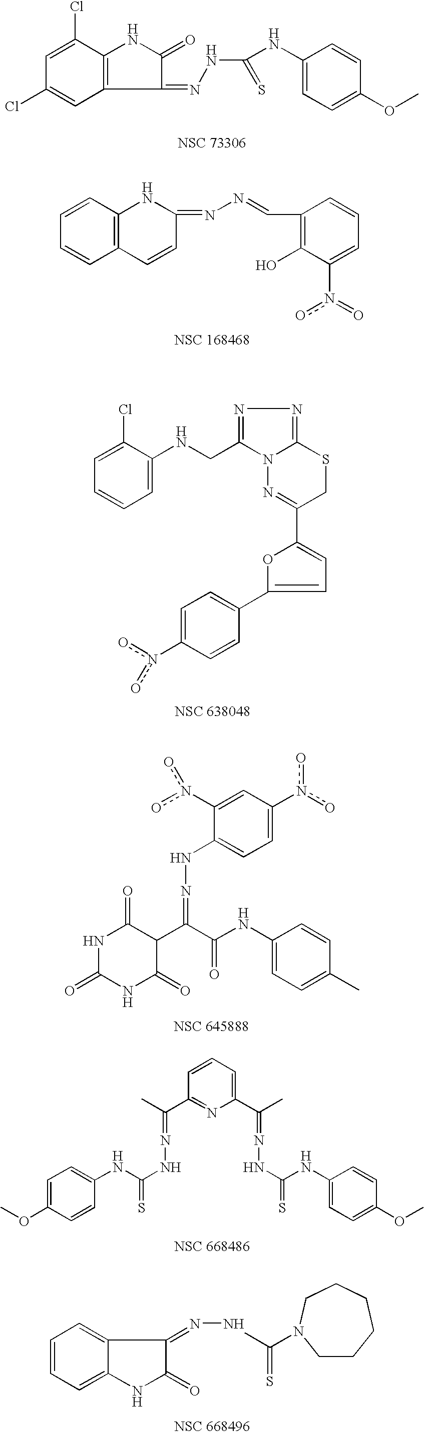

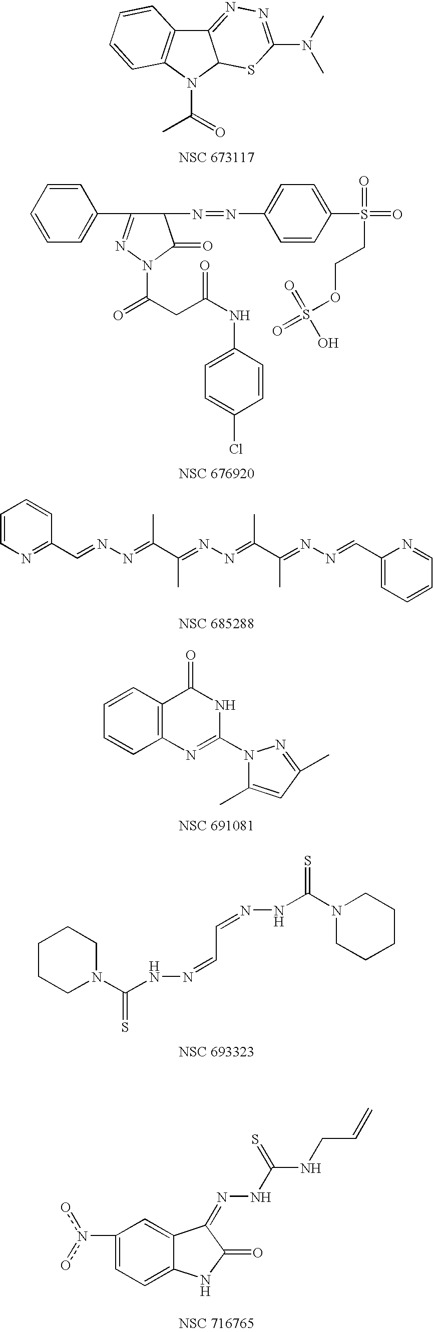

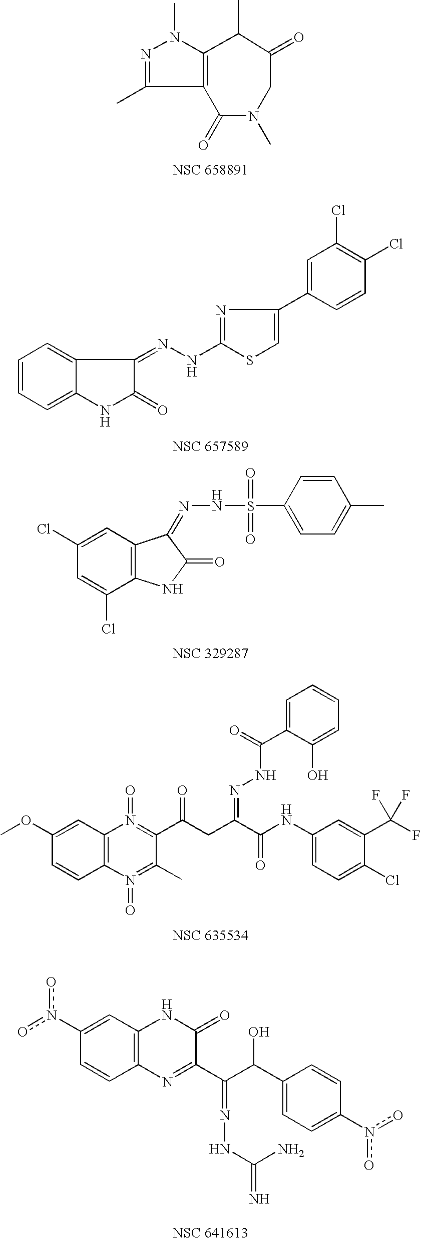

### Administration

An effective amount of one or more of the ABCB1 potentiated compounds of the present invention may be determined by one of ordinary skill in the art, and includes exemplary dosage amounts for a human of from about 0.05 to about 200 mg/kg/day. This dosage is typically administered in a single dose, but can be given in multiple doses. The compound(s) may be administered in a frequent regimen, e.g., daily, every two days for five doses, etc. or intermittently, e.g., every four days for three doses or every eight days for three doses. It will be understood that the specific dose level and frequency of administration for a given subject may be varied and will depend upon a variety of factors including, for example, the subject's age, body weight, general health, sex, diet and the like, and the mode of administration, the type of cancer or neoplastic condition, severity of the condition, and the type of other chemotherapeutic compounds that are being simultaneously administered.

The ABCB1 potentiated compounds are administered in pharmaceutical compositions containing an amount thereof effective for cancer therapy, and a pharmaceutically acceptable carrier. Such compositions may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation and/or called for by accepted pharmaceutical practice.

The ABCB1 potentiated compounds may be administered by any suitable means, for example, orally, such as in the form of tablets, capsules, granules or powders; sublingually; bucally, parenterally, such as by subcutaneous, intravenous, intramuscular, intracissternal, or intrathecal injection or infusion techniques (e.g., as sterile injectable aqueous or non-aqueous solutions or suspensions); nasally, such as by inhalation spray; topically, such as in the form of a cream or ointment; or rectally such as in the form of suppositories; in dosage unit formulations containing non-toxic, pharmaceutically acceptable vehicles or diluents. The subject compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The subject compounds may also be administered liposomally.

Suitable dosage forms for the ABCB1 potentiated compounds include, without intended limitation, an orally effective composition such as a tablet, capsule, solution or suspension containing about 0.1 to about 500 mg per unit dosage of an ABCB1 potentiated compound. They may be compounded in a conventional manner with a physiologically acceptable vehicle or carrier, excipient, binder, preservative, stabilizer, flavor, etc. The ABCB1 potentiated compounds can also be formulated in compositions such as sterile solutions or suspensions for parenteral administration. About 0.1 mg to about 500 mg of an ABCB1 potentiated compound may be compounded with a physiologically acceptable vehicle, carrier, excipient, binder preservative, stabilizer, etc., in a unit dosage form as called for by accepted pharmaceutical practice. The amount of active substance in these compositions or preparations is preferably such that a suitable dosage in the range indicated is obtained.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art Molded tablets, compressed tablets or freeze-dried tablets are exemplary forms that may be used. Exemplary compositions include those formulating the present compound(s) with fast dissolving diluents such as mannitol, lactose, sucrose and/or cyclodextrins. Also included in such formulations may be high molecular weight excipients such as celluloses (Avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g. Gantrez), and agents to control release such as polyacrylic acid copolymer (e.g. Carbopol 934). Lubricants, glidants, flavors, coloring agents and stabilizers may also be added for ease of fabrication and use.

Exemplary compositions for nasal aerosol or inhalation administration include solutions in saline, which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parentally acceptable diluents or solvents, such as Cremophor (polyoxyethylated caster oil surfactant), mannitol, 1,3-butanediol, water, Ringer's solution, Lactated Ringer's solution, an isotonic sodium chloride solution, or other suitable dispersing or wetting and suspending agents, including synthetic mono- or diglycerides, and fatty acids, including oleic acid. Exemplary compositions for rectal administration include suppositories, which may contain, for example, a suitable non-irritating excipient, such as cocoa butter, synthetic glyceride esters or polyethylene glycols, which are solid at ordinary temperature, but liquefy and/or dissolve in the rectal cavity to release the drug.

The ABCB1 potentiated compounds may be administered either alone or in combination with other chemotherapeutic agents or anti-cancer and cytotoxic agents and/or treatments useful in the treatment of cancer or other proliferative diseases. Especially useful are anti-cancer and cytotoxic drug combinations wherein the second drug chosen acts in a different manner or different phase of the cell cycle. Example classes of anti-cancer and cytotoxic agents include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone antagonists, octreotide acetate; microtubule-disruptor agents, such as ecteinascidins or their analogs and derivatives; and epothilones A-F or their analogs or derivatives; plant-derived products, such as vinca alkaloids, epipodophyllotoxins, and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The subject compounds may also be used in conjunction with radiation therapy. It is contemplated as an aspect of the invention that more than ABCB1 potentiated compound may be administered to a subject.

### Other Applications of the Invention

In principle, cytotoxic effect of compounds could be potentiated by other ABC transporters as well. Given the suggested role of ABCC1 and ABCG2 in clinical anticancer drug resistance, the invention relates to the identification of ABCC1- and ABCG2-potentiated compounds. The present invention also relates to novel methods of identifying substrates of ABC transporters and of identifying therapeutic compounds whose therapeutic activity is potentiated by expression of ABC transporters. The methods comprise the steps of determining the expression levels of one or more ABC transporters in a panel of cell lines, determining the level of therapeutic activity of one more test compounds on the panel of cell lines, comparing the level of therapeutic activity of a test compound on the panel of cell lines with the expression levels of at least one ABC transporter gene in the panel of cell lines, wherein a positive correlation between therapeutic activity and gene expression for a particular ABC transporter gene identifies the test compound as having a therapeutic activity that is potentiated by the ABC transporter and a negative correlation between therapeutic activity and gene expression for a particular ABC transporter gene identifies the test compound as a substrate of the ABC transporter.

In preferred embodiments of the invention the panel of cell lines comprises at least about 30, 40, 50, 55 and 60 cell lines, preferably, at least about 30, 40, 50, 55 and 60 tumor cell lines. Preferably, the panel of cell lines comprises at least about 30, 40, 50, 55, and 60 cell lines of the NCI-60, with or without additional tumor cell lines, and the therapeutic activity being assessed is anti-proliferative activity. Preferably, the therapeutic activity being assessed is anti-proliferative activity. As used herein, therapeutic activity refers to any effects on the cell lines that may be measured and that may be related to potential therapeutic activity of the test compound.

ABC gene expression levels may be determined in many different ways, including both the measurement of protein levels or RNA levels. Additionally, it is contemplated as an aspect of the invention that the level of ABC gene expression may not be determined de novo, but rather may be determined by consulting an existing set of data, such as for example, the data provided in the Examples herein.

Expression of ABC proteins may be measured in a semi-quantitative manner by methods known in the art such as gel electrophoresis or protein array techniques, ABC protein levels are preferably determined using a quantitative method such as an ELISA assays. Expression levels of ABC RNAs may be determined using a variety of techniques that are well known in the art, including Northern blot analysis, RNAse protection assays, and nucleic acid array technologies.

Preferably, the expression levels of the selected ABC genes are determined by means of RT-PCR, most preferably real time RT-PCR, since these techniques are sensitive and highly reproducible. For example, real time RT-PCR may be performed as described in the Examples herein or as described in U.S. Pat. No. 6,174,670. Sample preparation is one of the most critical aspects of quantitative PCR since isolation of high quality RNA is an important first step for the quantification of gene expression. Total cellular RNA is sufficient for analysis but contamination of DNA should be minimal. RNA sequences to be amplified may not only be derived from total cellular RNA but also from mRNA. Several mRNA isolation techniques are well known in the art.

Real time RT-PCR may be performed with a variety of different alternative detection formats that are well known in the art, including, for example, the following: (a) FRET Hybridization Probes; (b) TaqMan Hybridization Probes; (c) Molecular Beacons; (d) SyberGreen Format.

Having now generally described the invention, the same will be more readily understood through reference to the following examples, which are provided by way of illustration and are not intended to be limiting of the present invention unless specified.

### Example 1

### Correlations between ABC Gene Expression in Cancer Cells and Drug Sensitivities of the Cells

### Materials and Methods

### Purification of RNA

Total RNA is purified using the RNeasy kit (Qiagen), according to the manufacturer's instructions, as described by Scherf et al. (2000, Nature Genet. 24, 236-244). Aliquots of the RNA are stored at −70° C. The quality (purity and integrity) of the RNA samples are assessed via an Agilent 2100 Bioanalyzer with the RNA 6000 NanoLabChip reagent set (Agilent Technologies) and by assessment of the ribosomal RNA bands on a native agarose gel. The RNA is quantitated using a spectrophotometer.

### Quantitative RT-PCR

Expression levels are measured by real-time quantitative RT-PCR using the LightCycler RNA Amplification SYBR Green kit and a LightCycler machine (Roche Biochemicals, Indianapolis, Ind.). Specific oligonucleotide probes are designed for each of the ABC transporters using DNAStar Primer Select (DNASTAR Inc.), and they may be synthesized at Lofstrand Laboratories (Gaithersburg, Md.). When possible, the amplicons are designed to encompass exon-intron boundaries to avoid amplification of genomic DNA. Since the Syber Green assay detects accumulation of double stranded DNA, primers are selected (from a battery consisting of about 200 primers) that amplified a single product of the correct size. A list of the primers and corresponding gene reference/accession numbers for the ABC proteins is shown in Table 1 below. Table 1 shows a list of 47 ABC transporter genes, their accession numbers, and exemplary primers that may be used for real-time RT-PCR amplification of these genes.

TABLE 1

Primers for Real Time RT-PCR Amplification
of the ABC Genes

| | | Position | |
|---|---|---|---|
| | | of primer | Forward Oligo Sequence |
| # ABC | RefSeq # | on refseq | Reverse Oligo Sequence |
| ABCA1 | NM_005502 | 953-1157 | GCACTGAGGAAGATGCTGAAA |
| | | | (SEQ ID NO:1) |
| | | | AGTTCCTGGAAGGTCTTGTTCAC^{a} |
| | | | (SEQ ID NO:2) |
| ABCA2 | NM_001606 | 238-694 | CATCCCCCTGGTGCTGTTCTT |
| | | | (SEQ ID NO:3) |
| | | | GCTTGGGCCGTGCTATTGG |
| | | | (SEQ ID NO:4) |
| ABCA3 | NM_001089 | 437-939 | GCCCTCTTTACACTCAGTTTTCA |
| | | | (SEQ ID NO:5) |
| | | | GACGAGCAGTTGTCGTACCTAAT^{b} |
| | | | (SEQ ID NO:6) |
| ABCA4 | NM_000350 | 1361-1765 | TGGTCAAAGCCTGGGAAGAAGTA |
| | | | (SEQ ID NO:7) |
| | | | TCCAGGGATACATGTCAGGGAAT^{b} |
| | | | (SEQ ID NO:9) |
| ABCA5 | NM_018672 | 429-684 | GGGCCCAATGGTAGGAGGTAGAG |
| | | | (SEQ ID NO:9) |
| | | | TGAGGAATGGGCAAGGGAGGT |
| | | | (SEQ ID NO:10) |
| ABCA6 | NM_080284 | 4314-4630 | CCGTCAAGGGGCTCAGGAA |
| | | | (SEQ ID NO:11) |
| | | | GATGGCCACACGGTCACAC |
| | | | (SEQ ID NO:12) |
| ABCA7 | NM_019112 | 1491-2028 | CCCGGCCACGTGCGCATCAAAAT |
| | | | (SEQ ID NO:13) |
| | | | CCACCGCGAAGGCTGCCAAGAACA |
| | | | (SEQ ID NO:14) |
| ABCA8 | NM_007168 | 2099-2254 | AGTGCGCGGGCTCTTCTTTGT |
| | | | (SEQ ID NO:15) |
| | | | GTTTTCCTTCGCTTTTGGCTGATA |
| | | | (SEQ ID NO:16) |
| ABCA9 | NM_080293 | 581-1177 | CCCCATGATGAAAGAGCACAGAG |
| | | | (SEQ ID NO:17) |
| | | | AGGATCCCCCAAAAGACAATAAGG |
| | | | (SEQ ID NO:18) |
| ABCA10 | NM_080282 | 3455-3630 | ATGGCTCAGATGATCCCTCCTACA |
| | | | (SEQ ID NO:19) |
| | | | CTCCGTTTGAATAAGCTCCGTGAA |
| | | | (SEQ ID NO:20) |
| ABCA12 | XM_049831 | 3740-4021 | TCTCGCCGAAGTATATGGGATGTT |
| | | | (SEQ ID NO:21) |
| | | | GGCTTCGGGGAGATGTGATTG |
| | | | (SEQ ID NO:22) |
| ABCB1 | NM_000927 | 4313-4620 | TGACATTTATTCAAAGTTAAAAGCA |
| | | | (SEQ ID NO:23) |
| | | | TAGACACTTTATGCAAACATTTCAA |
| | | | (SEQ ID NO:24) |
| ABCB2 | NM_000593 | 613-1111 | AGGGCTGGCTGGCTGCTTTGA |
| | | | (SEQ ID NO:25) |
| | | | ACGTGGCCCATGGTGTTGTTAT |
| | | | (SEQ ID NO:26) |
| ABCB3 | NM_000544 | 849-1141 | ACGGCTGAGCTCGGATACCAC |
| | | | (SEQ ID NO:27) |
| | | | CCTCGGCCCCAAAACTGC |
| | | | (SEQ ID NO:28) |
| ABCB4 | NM_018850 | 3638-3933 | ACCGACTGTCTACGGTCCGAA |
| | | | (SEQ ID NO:29) |
| | | | TCCATCGGTTTCCACATCAAGG |
| | | | (SEQ ID NO:30) |
| ABCB5 | U66692 | 220-353 | TCTGGCCCCTCAAACCTCACC |
| | | | (SEQ ID NO:31) |
| | | | TTTCATACCGCCACTGCCAACTC |
| | | | (SEQ ID NO:32) |
| ABCB6 | NM_005689 | 2599-2880 | CAACCGCACCACCATCGTAGT |
| | | | (SEQ ID NO:33) |
| | | | AATAAGCCAGGGAAAGGAGACACA |
| | | | (SEQ ID NO:34) |
| ABCB7 | NM_004299 | 1589-1950 | TGGGTCAGGGAAAAGCACAATAG |
| | | | (SEQ ID NO:35) |
| | | | GGGGTCCTTCAAAATGGCTCTT |
| | | | (SEQ ID NO:36) |
| ABCB8 | NM_007188 | 2039-2372 | GGGCCCACTGCATTGTCGT |
| | | | (SEQ ID NO:37) |
| | | | CGGCCCCGGCTTTATTGT |
| | | | (SEQ ID NO:38) |
| ABCB9 | NM_019625 | 1799-2177 | GAGGGCCGGGTGGACTTTGAGAAT |
| | | | (SEQ ID NO:39) |
| | | | CAGTGGGCAGGCCGTAGGAGATGT |
| | | | (SEQ ID NO:40) |
| ABCB10 | NM_012089 | 1038-1556 | ATGGGCGATATCTACGGAAACTGA |
| | | | (SEQ ID NO:41) |
| | | | GGCGAGCTGGATAGGCAAAAT |
| | | | (SEQ ID NO:42) |
| ABCB11 | NM_003742 | 2102-2289 | AGGGAAATCAAGCTCTTAATGAAG |
| | | | (SEQ ID NO:43) |
| | | | ATAGGTAGACTTATGATCTACAACA |
| | | | (SEQ ID NO:44) |
| ABCC1 | NM_004996 | 119-1670 | AGTGGAACCCCTCTCTGTTTAAG |
| | | | (SEQ ID NO:45) |
| | | | CCTGATACGTCTTGGTCTTCATC^{b} |
| | | | (SEQ ID NO:46) |
| ABCC2 | NM_000392 | 3329-3531 | TCCTTGCGCAGCTGGATTACAT |
| | | | (SEQ ID NO:47) |
| | | | TCGCTGAAGTGAGAGTAGATTG |
| | | | (SEQ ID NO:48) |
| ABCC3 | NM_020038 | 2911-3180 | CAGAGAAGGTGCAGGTGACA |
| | | | (SEQ ID NO:49) |
| | | | CTAAAGCAGCATAGACGCCC |
| | | | (SEQ ID NO:50) |
| ABCC4 | NM_005845 | 3880-4124 | TGATGAGCCGTATGTTTTGC |
| | | | (SEQ ID NO:51) |
| | | | CTTCGGAACGGACTTGACAT |
| | | | (SEQ ID NO:52) |
| ABCC5 | NM_005688 | 1695-2261 | AGGGGCAAGAAAGAGAAGGTGAGG |
| | | | (SEQ ID NO:53) |
| | | | GAGGGGGTCGTCCAGGATGTAGAT |
| | | | (SEQ ID NO:54) |
| ABCC6 | NM_001171 | 3062-3492 | GGCCCGGGCATCCAGGTT |
| | | | (SEQ ID NO:55) |
| | | | TTTCATCTACGCGAGCATTGTTCT |
| | | | (SEQ ID NO:56) |
| ABCC7 | NM_000492 | 555-1029 | CATTTTTGGCCTTCATCACATT |
| | | | (SEQ ID NO:57) |
| | | | TGCCTTCCGAGTCAGTTTCAG |
| | | | (SEQ ID NO:58) |
| ABCC8 | NM_000352 | 3424-3619 | CTGCTAAACCGGATCATCCTAGCC |
| | | | (SEQ ID NO:59) |
| | | | CGAGGAACACAGGTGTGACATAGG |
| | | | (SEQ ID NO:60) |
| ABCC9 | NM_020298 | 1420-1556 | GCTACAAAGTTGGCAGAGGC |
| | | | (SEQ ID NO:61) |
| | | | TCCCAGGCATACAATTTTAGAAGT |
| | | | (SEQ ID NO:62) |
| ABCC10 | U66684 | 930-1234 | GGCTCCGGCAAGTCTTCCCTGTT |
| | | | (SEQ ID NO:63) |
| | | | AGATAGCTCCGGCCCCCTTCACC |
| | | | (SEQ ID NO:64) |
| ABCC11 | NM_033151 | 3025-3560 | CCACGGCCCTGCACAACAAG |
| | | | (SEQ ID NO:65) |
| | | | GGAATTGCCAAAAGCCACGAACA |
| | | | (SEQ ID NO:66) |
| ABCC12 | NM_033226 | 4195-4740 | CACCGCCTCTATGGACTCC |
| | | | (SEQ ID NO:67) |
| | | | TCAATCTCAGGCACTGGGGT |
| | | | (SEQ ID NO:68) |
| ABCD1 | NM_000033 | 2050-2293 | ACCAGGTGATCTACCCGGACTCAG |
| | | | (SEQ ID NO:69) |
| | | | CTCACGGCGCTGGTGCATTCATCC |
| | | | (SEQ ID NO:70) |
| ABCD2 | NM_005164 | 160-454 | TGGCCTGATTCGACCTCTCC |
| | | | (SEQ ID NO:71) |
| | | | GTCTGCAGCGTTTCTCTTCCACT |
| | | | (SEQ ID NO:72) |
| ABCD3 | NM_002858 | 121-421 | CTCGGCCTGCACGGTAAGAA |
| | | | (SEQ ID NO:73) |
| | | | TGGCAGCGATGAAGTTGAGTAAGT |
| | | | (SEQ ID NO:74) |
| ABCD4 | NM_005050 | 1266-1459 | GGATCTGAGCCTAAAGATCTCCGAG |
| | | | (SEQ ID NO:75) |
| | | | GGGTCCCGTCAGTGAAGAATGGC |
| | | | (SEQ ID NO:76) |
| ABCE1 | NM_002940 | 404-666 | GGTTGCCTATCCCTCGTCCAG |
| | | | (SEQ ID NO:77) |
| | | | TGTCCCCTTTGCCAGCCTTAG |
| | | | (SEQ ID NO:78) |
| ABCF1 | NM_001090 | 244-499 | ACAGGCTGGGGAAGAAGAGAAAGT |
| | | | (SEQ ID NO:79) |
| | | | CAGGGCTGCAAAAACATTACCAC |
| | | | (SEQ ID NO:80) |
| ABCF2 | NM_005692 | 1431-1753 | TAGGGCGTTACCATCAGCATTTAC |
| | | | (SEQ ID NO:81) |
| | | | GACCAGCATCATACCACCCTCAA |
| | | | (SEQ ID NO:82) |
| ABCF3 | U66685 | 381-637 | GGGGCATCAGACACGCTCAC |
| | | | (SEQ ID NO:83) |
| | | | GTTGGGGCAGGGCATAGTCAT |
| | | | (SEQ ID NO:84) |
| ABCG1 | NM_004915 | 976-1152 | CAGGAAGATTAGACACTGTGG |
| | | | (SEQ ID NO:95) |
| | | | GAAAGGGGAATGGAGAGAAGA |
| | | | (SEQ ID NO:86) |
| ABCG2 | NM_004827 | 266-646 | CCGCGACAGTTTCCAATGACCT |
| | | | (SEQ ID NO:87) |
| | | | GCCGAAGAGCTGCTGAGAACTGTA |
| | | | (SEQ ID NO:88) |
| ABCG4 | NM_022169 | 687-1050 | GGTCTGGATAGCGCCTCTTGTTTC |
| | | | (SEQ ID NO:89) |
| | | | ATGGGGCAGGGACCTCGTTCTTC |
| | | | (SEQ ID NO:90) |
| ABCG5 | NM_022436 | 2131-2352 | GCCGACTGTGCATGACTGCTCTG |
| | | | (SEQ ID NO:91) |
| | | | TTACATTCTTGGGTCCGCTCAG |
| | | | (SEQ ID NO:92) |
| ABCG8 | NM_022437 | 1718-1952 | CCGGGGGCTTCATGATAAACTT |
| | | | (SEQ ID NO:93) |
| | | | CTGAGGCCAATGACGATGAGGTA |
| | | | (SEQ ID NO:94) |

^{a}Kielar et al., 2001, Clin. Chem. 47(12):2089-2097.
^{a}Klucken et al., 2000, Proc. Natl. Acad. Sci. USA. 97(2):817-822.

RT-PCR is carried out on 150 ng total RNA, in the presence of 250 nM specific primers. Following reverse transcription (20 min at 50° C.), the PCR reaction consists of 45 cycles of denaturation (15 sec at 95° C.), annealing (30 sec at 58° C.), and elongation (30 sec at 72° C.). No-template (water) reaction mixtures are prepared as negative controls.

### Data Processing

During PCR amplification, fluorescence emission is measured and recorded in real time by the LightCycler. Crossing point values are calculated, using the LightCycler software package, by the Fit Points analysis method, with baseline fluorescence set at 1. The SyberGreen assay measures accumulation of double-stranded products, and the appearance of primer dimers limits quantitation at high cycle numbers. The specificity of amplified products is verified by melting-curve analysis and agarose gel electrophoresis (not shown). The raw results are expressed as number of cycles to reach the crossing point. If the desired product is not detected, the corresponding value is adjusted to crossing points indicating no expression. To assess the contribution of experimental artifacts, selected cell lines are assessed in replicate. The average pairwise correlation of replicate expression profiles is 0.96. The reproducibility of the measurements is confirmed by cluster analyses, which shows that replicates cluster tightly together.

Since the expression levels of housekeeping genes (glyceraldehyde-3-phosphate dehydrogenase (GAPDH), Porphobilinogen Deaminase (PBGD), tyrosine 3-monooxygenase/tryptophan 5-monooxygenase activation protein, and zeta polypeptide (YWHAZ) are found to be highly variable among the 60 cell lines (not shown; however, see Vandesompele et al., 2002, Genome Biol. 3, RESEARCH0034), they are not used as controls, and data are normalized with respect to the mean expression of the transporters. Finally, the values are mean-centered and multiplied by −1 to indicate expression values with reference to the mean expression of each ABC transporter across the 60 cell lines.

### Drug Database

More than 100,000 chemical compounds have been tested in the NCI-60 screen by the Developmental Therapeutics Program at the National Institutes of Health. The present analysis focuses on a subset consisting of 118 compounds whose mechanisms of action are putatively classifiable (Weinstein et al., 1992, Science 275:343-349) and a larger set of 1400 compounds that have been tested multiple times and whose screening data meet quality control criteria described by Scherf et al. (2000, Nature Genet. 24:236-244). Both sets are available at http://discover.nci.nih.gov. The two are combined to form a joint dataset that includes 1429 compounds.

### Statistical Analysis

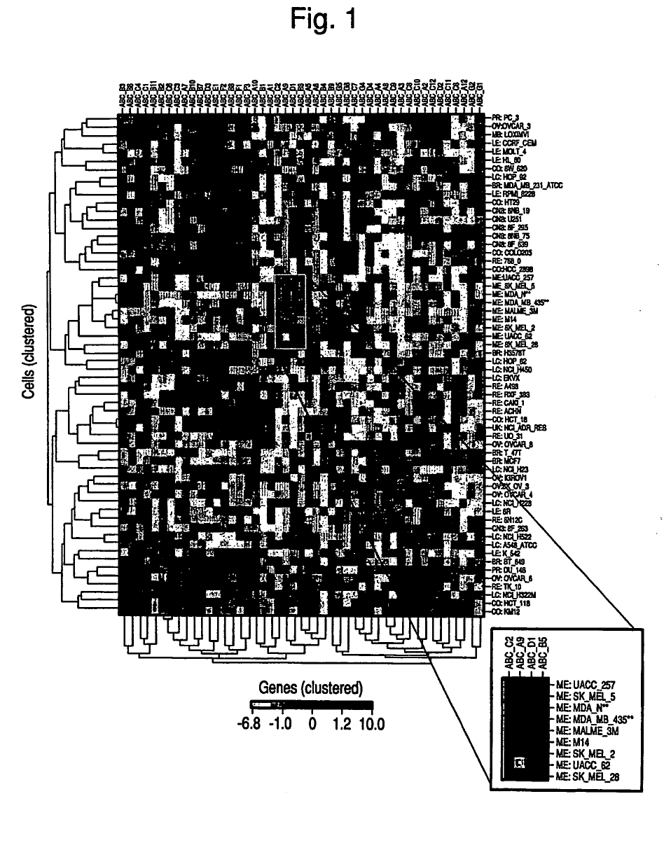

The statistical analyses are performed using the SAS software package, v8.2 (SAS Institute Inc, Cary, N.C.), and the R package (www.r-project.org). Two-dimensional agglomerative hierarchical cluster analysis, with average linkage algorithm and distance metric 1-r, where r is the Pearson correlation coefficient, is performed using the CIMminer tool (http://discover.nci.nih.gov) to group the 60 cell lines as well as the 47 ABC transporters based on the expression profiles. The resulting matrix of numbers is displayed in clustered image map form (Weinstein et al., 1997, Science 275:343-349) as shown graphically in FIG. 1, and numerically in Table 3.

To determine quantitatively how well the 47 genes cluster the cell lines by their tissues of origin, a statistical method is employed wherein the kappa statistic is used to indicate how well the observed clusters correspond to the nine tissue-of-origin classifications. For that calculation, one cell line, UK: NCI-ADR-RES, is excluded because it does not clearly fit into any of the usual categories. To identify which genes are, on average, significantly over- or under-expressed in cells from a given tissue of origin (in comparison with the rest of the cell lines), Monte Carlo permutation t-tests with 10,000 iterations are employed to compare, for each tissue, the within-tissue mean and the mean over all of the other tissue types (this approach avoids the assumption of normality and is suitable for small sample sizes). To control the overall false type 1 error rate, both a step-down procedure (Westfall and Young, 1993, Resampling-Based Multiple Testing: Examples and Methods for p-value Adjustment (New York: Wiley)) and a step-up procedure (Reiner et al., 2003, Bioinformatics 19:368-375) were employed to adjust for multiple testing of all 47 genes simultaneously. In the Benjamini-Hochberg procedure the p-values are computed in the standard way by permutation, assuming that all distributions are exchangeable: the number of values in the permuted data with correlations over a threshold, divided by the number of compounds and by the number of permutations. In this analysis, the False Discovery Rate (q-value) at which each compound would be declared was calculated using the step-up procedure for positively correlated test statistics (again true because all correlations being compared are computed against the same ABC gene): in this procedure the first q-value for the largest correlation is the Bonferroni-corrected p-value for that gene; then further q-values are calculated as qⱼ=max(pⱼ*1429/j, qⱼ₋₁). This procedure limits the expected proportion of false positives in the list 1, . . . , j to at most qⱼ. To narrow down the list of candidates based on correlation of the gene expression data for 47 ABC transporters and the extended list of 1429 drug activities measured in 60 cell lines (both centered around zero across the cell lines as well as across the expression values or the drug activities, respectively), the 95% and 99.99% bootstrap confidence intervals of Pearson correlation coefficients for all of the possible relationships is calculated (a total of 47×1429=67,163 correlation coefficients). The bootstrap confidence intervals are calculated using the empirical percentiles method with balanced re-sampling of 10,000 iterations. Balanced re-sampling forces each observation to appear exactly a number of times equal to the total number of iterations. The use of bootstrap re-sampling avoids parametric assumptions about the distributions of the variables and incorporated possible non-normal distributional characteristics. For 10,000 bootstrap iterations with 95% confidence interval, the component of resampling error has a standard error of no more than 0.002. In recognition of the multiple testing problem, a critical value of p<0.0001 is preferred.

### Drugs and Chemicals

The compounds designated by NSC numbers may be obtained from the Drug Synthesis and Chemistry Branch, Developmental Therapeutics Program, Division of Cancer Treatment and Diagnosis, National Cancer Institute. Colchicine and dimethyl sulphoxide (DMSO) may be purchased from Sigma Chemical Co. (St. Louis, Mo.), and PSC 833 may be obtained from Novartis Pharmaceuticals Corp. (East Hanover, N.J.).

### Analysis of Drug Sensitivity

Cell survival is measured by the MTT (3-(4,5-Dimethylthiazol-2-yl)-2,5-diphenyltetrazolium) Assay. Cells are seeded in 100 μl medium at a density of 5000 cells/well in 96 well plates, and serially diluted drug (with or without 2 μM PSC 833) is added the following day in 100 μl medium to give the indicated final concentration. Cells are then incubated for 72 hrs at 37° C. in 5% CO₂, and the MTT assay is performed according to the manufacturer's instructions (Molecular Probes, Eugene, Oreg.).

### Efflux Assay

Trypsinized cells are washed twice in phosphate-buffered saline (PBS). 5×10⁵ cells are pre-incubated for 5 min at 37° C. in Iscove's Modified Dulbecco's Medium (Quality Biologicals, Gaithersburg, Md.) with 0.5% dimethyl sulphoxide (DMSO), with or without 2 μM PSC 833. Compound NSC 634791 is then added to a final concentration of 1.74 μM, and the cells are incubated for 10 min at 37° C., then sedimented by centrifugation, and resuspended in PBS. Green fluorescence intensity is measured using a FacsCalibur flow cytometer equipped with a 488-nm argon laser (Becton Dickinson Biosciences, San Jose, Calif., USA). Acquisition of events is stopped at 10,000.

### Results

### ABC Gene Expression Analysis Across the NCI-60

Forty-eight (48) ABC proteins are coded by the human genome (see http://nutrigene.4t.com/humanabc.htm for a comprehensive database). The mRNA expression levels for 47 of the 48 ABC genes is profiled in 60 diverse cancer cell lines (the NCI-60) using real-time RT-PCR (expression data for ABCA13 was taken from the literature). The expression profiles of ABCC13 is not determined because its sequence is not known when the experiment is conducted. The real time RT-PCR results are presented below in Table 2.

Table 2 depicts, for each ABC gene tested, the values representing the expression level of that gene in 60 cell lines. The expression data of the 60 cell lines is presented in a matrix of 6 rows of 10 columns. Crossing point values are mean centered across the cells and across the transporters, then multiplied by −1 to reflect expression levels. The tested cell lines are (row, column (r,c)).

| (r, c) | Cell line |
|---|---|
| (1, 1) | BR-MCF7 |
| (1, 2) | UK-MCF7-ADR-RES |
| (1, 3) | BR-MDA-MB-231-ATCC |
| (1, 4) | ME-MDA-MB-435 |
| (1, 5) | ME-MDA-N |
| (1, 6) | BR-T-47D |
| (1, 7) | BR-BT-549 |
| (1, 8) | BR-HS578T |
| (1, 9) | CNS-SF-268 |
| (1, 10) | CNS-SF-295 |
| (2, 1) | CNS-SF-539 |
| (2, 2) | CNS-SNB-19 |
| (2, 3) | CNS-SNB-75 |
| (2, 4) | CNS-U251 |
| (2, 5) | CO-HCT-116 |
| (2, 6) | CO-HCT-15 |
| (2, 7) | CO-HT29 |
| (2, 8) | CO-KM12 |
| (2, 9) | CO-SW-620 |
| (2, 10) | CO-HCC-2998 |
| (3, 1) | CO-COLO205 |
| (3, 2) | OV-OVCAR-3 |
| (3, 3) | OV-OVCAR-4 |
| (3, 4) | OV-OVCAR-5 |
| (3, 5) | OV-OVCAR-8 |
| (3, 6) | OV-SK-OV-3 |
| (3, 7) | OV-IGROV1 |
| (3, 8) | RE-TK-10 |
| (3, 9) | RE-A498 |
| (3, 10) | RE-ACHN |
| (4, 1) | RE-786-0 |
| (4, 2) | RE-RXF-393 |
| (4, 3) | RE-CAKI-1 |
| (4, 4) | RE-UO-31 |
| (4, 5) | RE-SN12C |
| (4, 6) | PR-DU-145 |
| (4, 7) | PR-PC-3 |
| (4, 8) | ME-LOXIMVI |
| (4, 9) | ME-M14 |
| (4, 10) | ME-MALME-3M |
| (5, 1) | ME-SK-MEL-5 |
| (5, 2) | ME-SK-MEL-28 |
| (5, 3) | ME-SK-MEL-2 |
| (5, 4) | ME-UACC-257 |
| (5, 5) | ME-UACC-62 |
| (5, 6) | LC-A549-ATCC |
| (5, 7) | LC-EKVX |
| (5, 8) | LC-HOP-92 |
| (5, 9) | LC-NCI-H23 |
| (5, 10) | LC-NCI-H322M |
| (6, 1) | LC-NCI-H460 |
| (6, 2) | LC-NCI-H522 |
| (6, 3) | LC-HOP-62 |
| (6, 4) | LC-NCI-H226 |
| (6, 5) | LE-SR |
| (6, 6) | LE-MOLT-4 |
| (6, 7) | LE-HL-60 |
| (6, 8) | LE-K-562 |
| (6, 9) | LE-CCRF-CEM |
| (6, 10) | LE-RPMI-8226. |

TABLE 2
Expression of ABC Transporters in the NCI-60 cell lines
ABCA1

| | | | | | | | | | |
|---|---|---|---|---|---|---|---|---|---|
| −2.07 | 0.38 | 0.36 | 0.27 | −0.11 | −2.50 | 0.37 | −0.50 | −1.83 | −0.24 |
| 0.57 | −0.48 | 0.76 | −2.19 | −2.40 | 1.41 | 2.97 | −2.64 | 0.16 | −2.62 |
| −2.58 | 2.60 | −1.91 | −0.66 | 0.39 | −0.51 | −0.08 | 1.23 | 1.26 | 0.04 |
| 3.28 | 1.64 | 1.38 | 0.47 | 2.15 | −1.23 | 0.55 | −0.10 | −0.02 | 0.06 |
| 1.06 | 0.39 | −1.20 | −2.30 | 0.05 | 0.36 | −1.05 | 0.11 | −2.42 | 0.63 |
| 0.70 | 0.92 | −0.68 | 2.06 | 0.62 | 2.04 | 2.95 | −1.93 | −0.63 | 0.71 |

ABCA2

| | | | | | | | | | |
|---|---|---|---|---|---|---|---|---|---|
| 0.80 | 0.47 | −0.14 | 0.17 | 0.90 | 0.61 | −0.67 | 0.06 | −0.18 | 0.06 |
| −0.71 | −1.08 | 0.82 | −1.34 | −0.21 | −0.53 | 0.18 | 0.90 | −1.04 | −0.42 |
| −1.83 | 0.31 | 1.02 | 0.54 | 0.24 | 0.93 | −0.77 | 0.94 | −0.31 | −0.30 |
| 1.26 | 0.27 | 1.11 | −0.07 | −0.24 | 1.45 | 0.76 | −0.04 | 0.02 | −0.23 |
| −0.70 | −0.49 | 0.03 | 0.26 | −1.04 | 0.92 | −0.08 | −0.72 | −1.99 | 1.06 |
| −0.41 | 1.95 | −0.38 | 0.03 | −0.55 | −2.49 | 1.07 | −1.09 | −0.01 | 0.94 |

ABCA3

| | | | | | | | | | |
|---|---|---|---|---|---|---|---|---|---|
| 3.79 | 2.40 | 0.93 | −4.96 | −4.64 | 5.11 | 3.78 | −2.88 | 4.64 | −0.63 |
| −8.09 | 1.68 | −2.88 | −1.35 | −0.42 | 2.30 | −4.62 | 3.42 | −0.65 | −2.75 |
| −7.00 | −1.56 | 0.82 | 3.35 | 4.11 | 3.42 | 3.31 | 4.15 | 2.43 | 3.05 |
| −1.55 | 2.51 | −1.18 | 3.67 | 0.97 | 2.42 | 2.59 | −5.22 | −3.68 | −2.63 |
| −2.01 | −4.09 | −5.70 | −6.68 | −3.48 | 3.83 | 3.38 | −0.04 | 2.72 | −0.26 |
| 0.03 | 5.19 | −0.71 | 2.84 | 6.30 | −5.10 | −2.39 | 5.23 | −7.28 | 0.04 |

ABCA4

| | | | | | | | | | |
|---|---|---|---|---|---|---|---|---|---|
| 3.95 | −0.76 | −0.42 | −1.76 | −1.64 | 3.66 | −1.13 | −0.38 | 2.29 | −2.23 |
| −2.42 | −3.07 | −0.02 | 4.33 | 1.03 | −2.21 | −0.27 | −2.66 | −1.71 | −2.64 |
| −2.46 | 3.41 | 5.17 | −1.82 | 0.42 | 1.04 | 3.76 | −1.58 | −1.97 | −1.63 |
| −1.79 | 1.60 | 1.31 | −3.02 | 0.32 | −1.11 | 3.96 | −2.10 | −2.05 | −1.64 |
| −2.16 | −1.71 | −1.11 | 2.83 | −1.67 | −1.59 | 1.10 | 1.84 | 3.97 | −0.16 |
| −2.72 | −1.11 | −2.48 | 6.99 | 1.75 | 5.56 | −3.38 | −1.81 | 5.35 | −1.24 |

ABCA5

| | | | | | | | | | |
|---|---|---|---|---|---|---|---|---|---|
| 0.26 | 0.90 | 0.80 | 0.35 | −0.43 | 1.38 | 1.71 | 0.44 | 0.55 | −0.60 |
| 0.57 | −0.28 | −0.40 | 0.87 | −0.21 | 0.87 | −1.98 | −0.78 | 1.69 | 0.21 |
| −0.72 | −0.59 | 0.01 | 1.21 | −0.06 | 0.09 | −0.34 | −0.81 | −0.08 | −2.13 |
| −3.41 | 0.01 | −0.81 | −0.82 | −0.93 | 0.24 | 0.05 | −2.68 | 2.93 | 1.18 |
| 1.49 | 1.07 | −0.46 | 2.08 | −0.18 | −0.38 | −0.61 | −0.14 | 1.33 | −0.71 |
| 0.34 | −0.07 | 0.67 | 0.55 | −1.92 | 1.05 | 0.62 | −2.31 | −0.12 | −0.59 |

ABCA6

| | | | | | | | | | |
|---|---|---|---|---|---|---|---|---|---|
| −1.79 | 0.73 | −1.25 | −1.70 | −1.50 | −1.59 | 2.00 | 3.01 | −1.45 | −1.07 |
| −1.59 | −1.07 | −1.24 | −1.07 | −0.87 | −0.32 | −1.07 | −1.39 | −1.45 | −0.80 |
| −1.14 | −1.25 | −1.05 | −1.45 | −1.05 | −1.25 | 2.28 | −1.05 | −1.59 | −1.39 |
| −1.14 | −1.59 | −1.39 | −0.15 | −1.25 | 0.17 | 0.17 | −0.35 | 5.91 | 2.65 |
| 2.78 | 3.38 | −0.78 | −0.80 | 5.69 | 0.83 | −2.04 | −0.20 | 4.21 | −0.28 |
| 2.11 | 0.96 | 0.33 | 5.99 | −1.05 | 0.37 | −0.73 | 2.53 | −1.25 | −0.73 |

ABCA7

| | | | | | | | | | |
|---|---|---|---|---|---|---|---|---|---|
| −0.83 | 0.92 | 0.90 | −0.89 | −0.02 | 0.43 | −0.24 | −1.65 | −1.10 | 0.47 |
| −0.58 | 1.22 | 0.41 | 1.34 | 1.30 | 0.97 | 0.91 | −0.58 | −1.02 | 3.36 |
| 0.29 | −0.39 | −0.33 | 1.18 | −0.95 | −0.12 | −0.90 | 0.24 | 0.39 | −1.24 |
| 0.12 | 0.81 | −0.11 | −1.46 | 0.13 | 1.54 | −0.26 | 0.17 | 0.41 | −3.18 |
| −1.60 | 0.58 | −2.08 | −2.10 | −3.07 | 0.44 | 1.18 | 1.39 | −0.40 | 0.86 |
| −1.44 | −0.35 | −0.40 | 0.85 | −0.36 | −1.24 | 0.30 | 3.56 | 0.43 | 1.75 |

ABCA8

| | | | | | | | | | |
|---|---|---|---|---|---|---|---|---|---|
| 0.68 | −3.08 | −3.89 | 0.31 | 1.55 | −0.36 | −1.11 | −1.33 | 0.13 | 0.27 |
| −2.40 | −2.43 | −2.67 | −1.58 | −0.63 | −3.15 | −2.57 | 1.56 | −0.76 | −0.79 |
| −0.76 | −0.42 | 1.53 | −1.59 | 0.91 | 0.79 | −0.29 | 0.48 | 0.24 | −0.86 |
| −3.24 | −0.97 | 0.45 | 0.77 | 2.21 | −0.05 | 1.15 | 0.50 | −1.27 | −1.32 |
| 5.88 | −2.12 | 1.11 | 1.46 | 3.73 | 1.91 | −1.05 | −1.74 | −1.39 | 2.97 |
| 5.63 | 3.35 | 0.00 | −0.15 | 1.83 | −1.00 | −0.26 | 3.03 | 0.27 | 0.52 |

ABCA9

| | | | | | | | | | |
|---|---|---|---|---|---|---|---|---|---|
| −1.61 | −1.52 | −1.07 | 7.23 | 5.52 | −1.41 | −1.52 | 4.54 | −1.27 | −0.89 |
| −1.41 | −0.89 | −1.06 | −0.89 | −0.69 | −0.14 | −0.89 | −1.21 | −1.27 | −0.62 |
| −0.96 | −1.07 | −0.87 | −1.27 | −0.87 | −1.07 | 0.75 | −0.87 | −1.41 | −1.21 |
| −0.96 | −1.41 | −1.21 | 0.03 | −1.07 | 0.35 | 0.35 | −0.17 | 4.86 | 2.65 |
| 3.73 | 0.79 | 2.59 | 3.01 | −1.59 | 1.01 | −1.78 | −0.02 | −1.02 | −0.10 |
| −0.64 | 1.13 | 0.51 | 4.89 | −0.87 | −1.52 | −0.55 | −1.52 | −1.07 | −0.55 |

ABCA10

| | | | | | | | | | |
|---|---|---|---|---|---|---|---|---|---|
| 0.21 | 0.53 | 1.56 | −1.85 | −1.78 | 1.80 | 0.37 | −0.65 | −1.06 | −1.89 |
| −0.05 | −0.06 | −1.09 | −0.81 | 1.13 | 1.38 | 2.45 | 0.38 | 1.34 | 1.18 |
| −1.53 | −0.69 | −0.76 | 1.27 | −0.34 | 1.87 | 0.55 | 0.35 | −0.27 | 1.21 |
| −0.68 | 0.86 | −0.59 | −0.52 | 0.18 | 0.98 | −1.35 | −1.64 | 1.01 | 0.70 |
| −1.63 | −1.09 | −2.17 | 0.48 | −0.55 | 0.74 | −0.51 | 0.50 | 0.63 | −1.41 |
| −0.60 | 2.32 | −0.84 | 0.50 | −1.60 | 1.98 | 1.49 | −0.56 | 0.69 | −2.06 |

ABCA12

| | | | | | | | | | |
|---|---|---|---|---|---|---|---|---|---|
| 5.42 | −2.52 | −2.07 | −2.52 | −2.32 | 7.13 | −2.52 | −2.97 | −2.27 | 0.67 |
| 0.54 | 1.72 | 2.40 | −1.17 | 1.77 | −1.14 | 1.58 | 3.23 | −2.27 | −1.62 |
| 6.90 | −2.07 | −1.87 | −2.27 | −1.87 | −2.07 | −2.97 | −1.87 | 5.07 | 3.85 |
| 3.85 | 7.36 | 4.10 | 3.21 | −2.07 | −0.65 | −0.65 | −1.17 | −2.02 | −2.70 |
| −2.77 | 2.27 | −1.60 | −0.29 | 2.80 | 0.01 | 0.86 | 0.18 | −1.06 | −1.10 |
| −0.81 | 0.13 | −0.49 | 2.77 | −1.87 | −2.52 | −1.55 | −2.52 | −2.07 | −1.55 |

ABCA13

| | | | | | | | | | |
|---|---|---|---|---|---|---|---|---|---|
| −0.01 | −0.83 | −0.82 | 0.48 | NA | 0.30 | −0.75 | −0.97 | −0.91 | 0.64 |
| 1.24 | 1.50 | 0.26 | 1.38 | −0.55 | −0.95 | −0.34 | −0.32 | −0.21 | −0.71 |
| −0.25 | 0.02 | −0.91 | −0.18 | −0.47 | −0.24 | 0.20 | 0.70 | −0.55 | −1.05 |
| −0.70 | −0.52 | −0.44 | 0.20 | −0.79 | 1.70 | −0.50 | −0.75 | 0.71 | 0.75 |
| 0.76 | −0.22 | 0.15 | 0.41 | 0.45 | −0.66 | 0.70 | 0.78 | −0.01 | 0.73 |
| 0.38 | −0.66 | −0.14 | −0.13 | 2.19 | 0.68 | −0.36 | −0.14 | −0.02 | −0.24 |

ABCB1

| | | | | | | | | | |
|---|---|---|---|---|---|---|---|---|---|
| −2.30 | 12.28 | −1.70 | −1.26 | −1.14 | −1.87 | −0.62 | 0.13 | −1.94 | 2.47 |
| −1.91 | −1.98 | −3.05 | −2.09 | −2.39 | 11.08 | −1.86 | −1.54 | 3.19 | −2.13 |
| −1.95 | −2.64 | −2.63 | −2.39 | 1.94 | −1.48 | 1.91 | −1.08 | 4.31 | 3.69 |
| 2.84 | −1.22 | 8.24 | 4.68 | −2.16 | −0.60 | −2.50 | −1.59 | 2.74 | −1.13 |
| 1.31 | −1.21 | −0.60 | −1.67 | −1.16 | −1.08 | 3.58 | −2.03 | −1.79 | −1.82 |
| 3.05 | −0.60 | −1.97 | −2.46 | −1.88 | −1.74 | 0.53 | 0.01 | 1.92 | −0.73 |

ABCB2

| | | | | | | | | | |
|---|---|---|---|---|---|---|---|---|---|
| −0.84 | 0.07 | −0.01 | −1.90 | −1.38 | −2.83 | 1.82 | 1.78 | 1.70 | −1.72 |
| 3.03 | −1.63 | 3.90 | −2.20 | 1.47 | −0.48 | 2.31 | −1.29 | −0.97 | 0.80 |
| 0.51 | −0.54 | −0.38 | −1.15 | −0.44 | 0.59 | −0.10 | 1.46 | −2.78 | −0.89 |
| 1.33 | 1.37 | 0.87 | −1.27 | −0.77 | −0.63 | 0.54 | 3.54 | −1.29 | −0.48 |
| −0.21 | −3.38 | −0.49 | 0.92 | 0.52 | 1.79 | −0.95 | −1.89 | −2.32 | −0.70 |
| −0.41 | −0.22 | 1.40 | 0.80 | 0.96 | 0.22 | 0.69 | −0.55 | 0.11 | 2.58 |

ABCB3

| | | | | | | | | | |
|---|---|---|---|---|---|---|---|---|---|
| −1.23 | −0.42 | 1.15 | −0.33 | 1.22 | −1.39 | 0.38 | 0.34 | −0.66 | 1.78 |
| 0.69 | −1.11 | 1.59 | 3.72 | 0.16 | −0.13 | 0.89 | 1.23 | −0.70 | 0.41 |
| −1.00 | 0.39 | 0.20 | 0.07 | −0.36 | −0.85 | −0.03 | 0.16 | 0.20 | −0.45 |
| 2.29 | −2.13 | −1.19 | 1.48 | 0.34 | −0.40 | −0.69 | 0.88 | −2.34 | −1.69 |
| −0.05 | −2.25 | 0.41 | −1.90 | 0.55 | 2.39 | −1.19 | −0.28 | −0.43 | 0.22 |
| −1.17 | 0.32 | −0.60 | 0.35 | 1.89 | 1.68 | 0.34 | −1.32 | −1.09 | −0.33 |

ABCB4

| | | | | | | | | | |
|---|---|---|---|---|---|---|---|---|---|
| 4.34 | 7.61 | −2.46 | −0.45 | −0.93 | 1.02 | −2.73 | −3.72 | −2.05 | 5.66 |
| 1.00 | 3.21 | −4.15 | 5.41 | −3.25 | 1.86 | 1.08 | −3.22 | 3.11 | −2.66 |
| −5.35 | −0.78 | −2.03 | 1.84 | 3.16 | −5.94 | −4.52 | 0.01 | −0.31 | −0.73 |
| −2.76 | 2.04 | 0.51 | 4.59 | −3.56 | −0.48 | −3.08 | 0.07 | 4.98 | 5.07 |
| −0.25 | −1.73 | 10.01 | 3.18 | −0.49 | −2.76 | 0.83 | −0.31 | 3.23 | −5.78 |
| −0.69 | −1.16 | 3.78 | −4.35 | −0.49 | 0.23 | −2.44 | 0.57 | −0.45 | −2.36 |

ABCB5

| | | | | | | | | | |
|---|---|---|---|---|---|---|---|---|---|
| −0.50 | −1.04 | −0.34 | 5.35 | 2.28 | −1.19 | −0.59 | −1.71 | −0.27 | −0.17 |
| −0.38 | 1.01 | 0.40 | 0.04 | −0.22 | −0.76 | −0.76 | −0.52 | −0.07 | −0.48 |
| −0.48 | −1.16 | −0.86 | −0.38 | −1.04 | −0.61 | −0.77 | −0.35 | 0.00 | −1.24 |
| −0.36 | −1.33 | −1.92 | −0.07 | 0.43 | −0.97 | −0.16 | −1.06 | −1.11 | 3.41 |
| 3.71 | 3.29 | 2.47 | 7.67 | 2.05 | 0.18 | −1.90 | −1.10 | −2.09 | −0.17 |
| −1.60 | 0.45 | −0.73 | −1.08 | 1.00 | −1.23 | −0.75 | −0.77 | 1.12 | −0.54 |

ABCB6

| | | | | | | | | | |
|---|---|---|---|---|---|---|---|---|---|
| −0.02 | −0.29 | −0.19 | 0.55 | 0.28 | 0.37 | −0.26 | 1.37 | 0.22 | 1.14 |
| 1.52 | 2.17 | 0.79 | 2.25 | −0.09 | −0.35 | −0.38 | −0.41 | 0.23 | 1.90 |
| −0.61 | −0.09 | −0.36 | −0.02 | −0.86 | −1.73 | 0.87 | −0.19 | 1.09 | −0.90 |
| 0.58 | 0.40 | −1.35 | −0.78 | 0.00 | 0.15 | −0.75 | −0.34 | −0.23 | −1.13 |
| 1.57 | −0.13 | −0.47 | 0.65 | 0.39 | 1.54 | −0.70 | −0.88 | −1.16 | 0.21 |
| −0.18 | 0.73 | −0.64 | −0.66 | −1.22 | −1.85 | −0.78 | 0.29 | 0.64 | −1.90 |

ABCB7

| | | | | | | | | | |
|---|---|---|---|---|---|---|---|---|---|
| −2.26 | 0.16 | 0.43 | −0.86 | −1.63 | −2.25 | 0.45 | 0.00 | −0.51 | −0.96 |
| 0.81 | 0.25 | 1.07 | 1.05 | −0.12 | 1.27 | 1.09 | −0.07 | 2.61 | 0.87 |
| 1.71 | 1.77 | −0.13 | −0.07 | −1.33 | −0.79 | −0.24 | −0.32 | −0.43 | −1.24 |
| 2.49 | 0.55 | −0.24 | −1.44 | −1.45 | −0.53 | −0.41 | 0.39 | −1.07 | −0.77 |
| −0.41 | −0.88 | −0.48 | −0.60 | −2.79 | −0.60 | 10.35 | 0.32 | −0.26 | −0.53 |
| 0.05 | −2.18 | 0.03 | −2.10 | −1.15 | 0.50 | 2.07 | 0.02 | −0.18 | 0.98 |

ABCB8

| | | | | | | | | | |
|---|---|---|---|---|---|---|---|---|---|
| −1.52 | 0.77 | −0.06 | 0.13 | −0.75 | −1.85 | 0.36 | 0.96 | 1.15 | 0.56 |
| 1.97 | 0.71 | 0.67 | 0.68 | −0.86 | −0.50 | −0.38 | −1.01 | −0.84 | 0.89 |
| 0.98 | −0.87 | −1.12 | −1.05 | −3.00 | −1.09 | −1.84 | 1.39 | 0.70 | 1.14 |
| 0.26 | 2.11 | 2.97 | 1.25 | −1.04 | 0.56 | 0.33 | 0.19 | 0.21 | 0.85 |
| 0.32 | −0.15 | −0.11 | 0.26 | −2.46 | −2.96 | −0.16 | 0.65 | 0.37 | 1.63 |
| −0.96 | −2.00 | 1.90 | −0.93 | −0.24 | 1.55 | −0.24 | 0.66 | −0.99 | −0.18 |

ABCB9

| | | | | | | | | | |
|---|---|---|---|---|---|---|---|---|---|
| 0.27 | 1.68 | 0.81 | 0.52 | 0.83 | −0.24 | −0.58 | −3.86 | −0.98 | 1.18 |
| 2.09 | 0.92 | 1.07 | 1.63 | 0.43 | 0.60 | 0.70 | 0.05 | −0.51 | 1.11 |
| 1.02 | 1.56 | −0.48 | 0.22 | −0.69 | −0.72 | −0.09 | −0.13 | −0.83 | 0.45 |
| −0.76 | 0.40 | 0.18 | 0.59 | 0.26 | 0.30 | −0.84 | −0.67 | 0.37 | 1.18 |
| 0.32 | −0.58 | 1.52 | −0.75 | 1.08 | 0.22 | −1.07 | 0.84 | 0.45 | −0.21 |
| −1.19 | −1.55 | −0.21 | −0.43 | −1.19 | −1.17 | −2.77 | 0.15 | −1.77 | −0.76 |

ABCB10

| | | | | | | | | | |
|---|---|---|---|---|---|---|---|---|---|
| −1.66 | −0.37 | 1.09 | −0.79 | −0.75 | 0.21 | 0.41 | 0.36 | 0.46 | −0.34 |
| −0.83 | 0.33 | 1.00 | −0.62 | −0.34 | 2.41 | 1.30 | 0.11 | 1.25 | 0.76 |
| 0.52 | −0.82 | −1.10 | 0.33 | −1.48 | 0.54 | 0.22 | 0.01 | −0.45 | 0.25 |
| 1.40 | 0.52 | 0.31 | −5.22 | −0.71 | 0.07 | −0.40 | −0.58 | −0.70 | −0.75 |
| −0.18 | 0.05 | −1.37 | −0.08 | −0.59 | −1.09 | 1.16 | 1.70 | −0.53 | 0.28 |
| 0.97 | −0.41 | 0.20 | −1.78 | −0.92 | −0.42 | 3.09 | 0.97 | 1.19 | 1.82 |

ABCB11

| | | | | | | | | | |
|---|---|---|---|---|---|---|---|---|---|
| 0.61 | −0.85 | −1.12 | −0.68 | −0.56 | −1.29 | −0.04 | 0.71 | −1.36 | −1.14 |
| 3.01 | 4.82 | 2.76 | 3.01 | −1.81 | −1.12 | 4.79 | −1.57 | −2.25 | 2.71 |
| 1.28 | 4.05 | −0.01 | −1.81 | −1.45 | −0.90 | 1.08 | −0.50 | −2.32 | −0.55 |
| −1.98 | −1.87 | −0.91 | −2.17 | −1.58 | −0.02 | 8.11 | 3.77 | −0.96 | −0.55 |
| −1.07 | −0.63 | −0.02 | −1.00 | −0.58 | −0.50 | −2.34 | −1.45 | −1.21 | −1.24 |
| 2.28 | −0.02 | 0.92 | −1.88 | −1.29 | −1.16 | 2.42 | 1.16 | −1.60 | −0.15 |

ABCC1

| | | | | | | | | | |
|---|---|---|---|---|---|---|---|---|---|
| −0.78 | 0.18 | −0.25 | −0.95 | −1.03 | −2.48 | −0.60 | −0.21 | 0.05 | −0.51 |
| 1.70 | −0.41 | 0.01 | 0.62 | 0.28 | −0.20 | 0.59 | 0.39 | 0.03 | −0.05 |
| 0.94 | 0.72 | −1.08 | 0.01 | 1.09 | 1.91 | 0.56 | −0.08 | −0.84 | −0.13 |
| 0.18 | −1.63 | −0.23 | −0.07 | −0.22 | 0.65 | 0.06 | 0.17 | −1.31 | −0.96 |
| −1.14 | −0.31 | −1.60 | 0.03 | 0.38 | −0.56 | −0.04 | −0.44 | 0.24 | 1.35 |
| 0.41 | 0.70 | 0.84 | 0.65 | −0.19 | 0.46 | 0.82 | 1.12 | 0.60 | 0.55 |

ABCC2

| | | | | | | | | | |
|---|---|---|---|---|---|---|---|---|---|
| −1.12 | −2.61 | −2.84 | 4.47 | 4.49 | −4.46 | 0.15 | −0.01 | −2.03 | 0.05 |
| −0.29 | −3.25 | −1.00 | −1.19 | 4.85 | −4.11 | −5.64 | 0.53 | 2.70 | 4.69 |
| 3.18 | −1.31 | −0.83 | −1.83 | −3.19 | −1.36 | −0.19 | −4.00 | 5.29 | −1.93 |
| −1.89 | −0.75 | −0.44 | −1.74 | −1.98 | 1.58 | −1.01 | −0.25 | 5.62 | 3.84 |
| 6.05 | 3.39 | 3.09 | 5.39 | 1.17 | 0.16 | −1.95 | −1.79 | 1.85 | 4.08 |
| 7.22 | −4.28 | 0.94 | 0.27 | −2.97 | −1.03 | −1.41 | −4.27 | −0.49 | −5.62 |

ABCC3

| | | | | | | | | | |
|---|---|---|---|---|---|---|---|---|---|
| −5.06 | −4.83 | 1.41 | −2.84 | −1.27 | 0.18 | −0.36 | 1.67 | −3.52 | 1.03 |
| 1.42 | 3.13 | 1.35 | 3.09 | 0.77 | −1.74 | 4.24 | 1.47 | −2.17 | 2.68 |
| 4.44 | −2.31 | −2.36 | 3.30 | −3.61 | 3.35 | −1.81 | 4.02 | 1.82 | 2.86 |
| 2.73 | 0.85 | −0.06 | 2.19 | −0.66 | 2.85 | 1.00 | −1.16 | −3.13 | −2.72 |
| −3.24 | −0.27 | −2.19 | −3.25 | −2.74 | −0.40 | 3.39 | 1.35 | −3.38 | 3.95 |
| 1.92 | −0.80 | 0.01 | 3.11 | −0.53 | −1.70 | −2.13 | −2.14 | −2.19 | −1.01 |

ABCC4

| | | | | | | | | | |
|---|---|---|---|---|---|---|---|---|---|
| −2.77 | −2.40 | 0.80 | 1.20 | 0.43 | −13.62 | 1.70 | −0.63 | −0.79 | 0.69 |
| 1.03 | −1.52 | 0.21 | −0.83 | 0.43 | −0.39 | 0.21 | 0.72 | 0.23 | −1.83 |
| −0.12 | −0.50 | 0.27 | 2.63 | 0.39 | 1.92 | 1.27 | −0.06 | 1.08 | 0.52 |
| −0.05 | 1.47 | −0.38 | 0.81 | 0.96 | 0.90 | −0.03 | 1.97 | −0.20 | −1.04 |
| 2.09 | 1.43 | −0.72 | 1.17 | 1.13 | 1.69 | 0.49 | −0.25 | −0.71 | −1.00 |
| −2.60 | −0.70 | −0.40 | 1.10 | −0.14 | −0.99 | 1.74 | 1.07 | −0.31 | 1.25 |

ABCC5

| | | | | | | | | | |
|---|---|---|---|---|---|---|---|---|---|
| 2.44 | 1.14 | −2.67 | −1.67 | −1.54 | −1.60 | 0.46 | 2.43 | −5.36 | −4.85 |
| 2.08 | −1.42 | 0.90 | −1.54 | 0.58 | 0.24 | −2.24 | −0.78 | −0.20 | 2.25 |
| −0.41 | 0.65 | 0.56 | 0.44 | 0.89 | 0.13 | 2.51 | 1.96 | −4.19 | −1.08 |
| 0.38 | −0.30 | 0.07 | 0.73 | −2.74 | −0.18 | 1.32 | 3.53 | 0.40 | 0.74 |
| 0.91 | 0.56 | −0.57 | 4.42 | 3.96 | 0.42 | −4.74 | −2.33 | −1.38 | 0.51 |
| −0.45 | 2.03 | 1.67 | −2.57 | −4.81 | 1.85 | 1.30 | 3.94 | 1.60 | −0.39 |

ABCC6

| | | | | | | | | | |
|---|---|---|---|---|---|---|---|---|---|
| 0.91 | 1.34 | 1.73 | −0.16 | 0.56 | 3.51 | −0.78 | −0.03 | 0.84 | −0.50 |
| 2.29 | −2.72 | 1.79 | −2.25 | 0.33 | 3.83 | −2.74 | 0.74 | 1.13 | −2.29 |
| 3.88 | −2.79 | −2.79 | −0.61 | 2.20 | −1.63 | −3.27 | 2.20 | −0.01 | 3.93 |
| 0.88 | 0.09 | 2.58 | 2.19 | 4.27 | −0.76 | −2.66 | −1.75 | −1.70 | −1.29 |
| −1.81 | 1.71 | −0.76 | −1.82 | −1.32 | 1.13 | 0.81 | 2.65 | −1.18 | −1.98 |
| −2.37 | −0.76 | −2.13 | −1.86 | 0.00 | −1.90 | 3.52 | −1.46 | −0.08 | −0.89 |

ABCC7

| | | | | | | | | | |
|---|---|---|---|---|---|---|---|---|---|
| −1.37 | −4.36 | 2.07 | −0.02 | 1.20 | −1.65 | −0.43 | −0.15 | 1.78 | 2.22 |
| 0.19 | 3.53 | −1.12 | 0.80 | −0.52 | −0.93 | 4.04 | 1.89 | 0.23 | 4.55 |
| 0.52 | −1.65 | 3.65 | −0.90 | −0.28 | 0.29 | −0.43 | −0.46 | −0.81 | −1.77 |
| 0.64 | −1.46 | −1.17 | −2.73 | 0.85 | −1.77 | −2.87 | −0.68 | −0.06 | −0.36 |
| −1.98 | 0.90 | −1.72 | −4.59 | −0.47 | −0.43 | −2.04 | 0.72 | −2.84 | −1.73 |
| −2.90 | 1.46 | −2.19 | −1.13 | 1.83 | 7.98 | 0.77 | 1.96 | 3.42 | 2.49 |

ABCC8

| | | | | | | | | | |
|---|---|---|---|---|---|---|---|---|---|
| 2.18 | −1.05 | −0.60 | −1.05 | −0.85 | −0.94 | −1.05 | −1.50 | 5.98 | −0.42 |
| −0.94 | −0.42 | −0.59 | −0.42 | −0.22 | 0.33 | −0.42 | −0.74 | 2.11 | −0.15 |
| −0.49 | −0.60 | −0.40 | 2.33 | −0.40 | 2.05 | 4.99 | −0.40 | −0.94 | −0.74 |
| −0.49 | −0.94 | −0.74 | 0.50 | −0.60 | 0.82 | 0.82 | 0.30 | −0.55 | −1.23 |
| −1.30 | −1.64 | −0.13 | −0.15 | −1.12 | 1.48 | −1.39 | 0.45 | 3.09 | 0.37 |
| 0.11 | 1.60 | 0.98 | −1.64 | −0.40 | −1.05 | −0.08 | −1.05 | −0.60 | −0.08 |

ABCC9

| | | | | | | | | | |
|---|---|---|---|---|---|---|---|---|---|
| 0.48 | −2.02 | −2.27 | −1.32 | 2.03 | 1.22 | −2.61 | 2.82 | 1.67 | −0.61 |
| −2.83 | −2.50 | −2.20 | −1.48 | 0.50 | −0.84 | −1.78 | 0.02 | −1.41 | −3.65 |
| −3.99 | −2.06 | 1.37 | −0.51 | 1.23 | 1.47 | 1.05 | 0.87 | 0.80 | 5.09 |
| −3.99 | −0.13 | 1.79 | −3.01 | 4.47 | −0.25 | 0.61 | 0.80 | −2.85 | −0.85 |
| −1.03 | 7.59 | −3.63 | −3.65 | 1.15 | −2.02 | 0.31 | 0.06 | −0.40 | −3.13 |
| 8.23 | −1.90 | 1.24 | 3.39 | 8.14 | −0.97 | −1.49 | 1.73 | 0.07 | 1.19 |

ABCC10

| | | | | | | | | | |
|---|---|---|---|---|---|---|---|---|---|
| 0.00 | 3.20 | −1.23 | −0.94 | 0.37 | −0.73 | 0.70 | 1.58 | 0.11 | 0.38 |
| 0.21 | −0.39 | 0.11 | 1.22 | 4.44 | −0.99 | 0.03 | 0.51 | 0.39 | −4.39 |
| −3.17 | 0.35 | 1.25 | −0.12 | 0.98 | 1.09 | −1.23 | 1.50 | −2.76 | 0.97 |
| 0.60 | 1.66 | 0.42 | 2.00 | −1.22 | −0.28 | 1.05 | 0.62 | −0.20 | −0.78 |
| −1.43 | −0.93 | 0.32 | −1.16 | −2.25 | 2.00 | 0.80 | −1.20 | −1.06 | 1.30 |
| −1.14 | 0.17 | 0.28 | −0.28 | −0.91 | −0.46 | −0.08 | 0.00 | −0.73 | −0.53 |

ABCC11

| | | | | | | | | | |
|---|---|---|---|---|---|---|---|---|---|
| 1.77 | −0.14 | −2.30 | −1.85 | −1.73 | 16.67 | −1.21 | −0.46 | −0.09 | −2.32 |
| −2.51 | 0.49 | 0.03 | −2.68 | 1.13 | −2.29 | −3.17 | 0.68 | 2.77 | −0.65 |
| 5.80 | 1.11 | 2.91 | 1.13 | −2.62 | −2.07 | 1.27 | −1.67 | −0.34 | −1.72 |
| −1.07 | −1.00 | −1.79 | −2.22 | 0.53 | 0.02 | 0.45 | 2.51 | −0.15 | −1.14 |
| 1.94 | −1.80 | −1.19 | 0.62 | 1.19 | −1.67 | 4.78 | 0.24 | 1.19 | 2.32 |
| −0.69 | −1.19 | 0.70 | −2.10 | −0.94 | −1.70 | 2.23 | −1.90 | −2.78 | −1.32 |

ABCC12

| | | | | | | | | | |
|---|---|---|---|---|---|---|---|---|---|
| 1.15 | −1.45 | 0.12 | −0.66 | 0.70 | 1.64 | −0.30 | −1.34 | 0.81 | 0.39 |
| −1.05 | 1.24 | −0.28 | −1.18 | 0.83 | −0.81 | −0.63 | 1.86 | −0.44 | −0.25 |
| −1.23 | −0.23 | 2.44 | 0.11 | 2.51 | 2.07 | 0.94 | 0.35 | 0.14 | 0.44 |
| −1.25 | −0.80 | −0.40 | 1.32 | 1.27 | −0.22 | 0.43 | −0.05 | −1.28 | −2.14 |
| −0.46 | −1.54 | −0.29 | −1.99 | 0.12 | 1.09 | −0.40 | −1.24 | −1.97 | 1.90 |
| −1.14 | 2.14 | −0.23 | −0.43 | 0.98 | −0.98 | −0.44 | −1.75 | 1.24 | 0.60 |

ABCD1

| | | | | | | | | | |
|---|---|---|---|---|---|---|---|---|---|
| −0.26 | −3.96 | 2.72 | 4.12 | 4.55 | 1.29 | −0.47 | 1.44 | 1.33 | 0.19 |
| 1.63 | 0.50 | 1.07 | 1.63 | −1.13 | −3.24 | −1.06 | −2.88 | −1.13 | −1.75 |
| −0.19 | −1.01 | −0.05 | −0.97 | −2.00 | 0.41 | −1.69 | −1.95 | 0.26 | −2.35 |
| −1.66 | −0.78 | −2.02 | −2.04 | 0.47 | −1.83 | 0.91 | −1.27 | 1.51 | 3.17 |
| 0.58 | 4.25 | 2.33 | 2.26 | 1.10 | −0.49 | 0.10 | 2.37 | −0.19 | −1.48 |
| −2.21 | 1.41 | −1.44 | 1.72 | 1.28 | −0.26 | 0.21 | −0.62 | −1.61 | −0.79 |

ABCD2

| | | | | | | | | | |
|---|---|---|---|---|---|---|---|---|---|
| 0.97 | −0.39 | −0.88 | −0.55 | 0.87 | 0.54 | −0.07 | −1.21 | 0.56 | 0.09 |
| −0.26 | 0.78 | 0.34 | −0.84 | −0.40 | −0.59 | −0.24 | 1.42 | 0.01 | −1.70 |
| −0.57 | −0.14 | 2.02 | 0.43 | 1.31 | 1.75 | −0.16 | 1.00 | −1.35 | −0.04 |
| −0.38 | −0.20 | −0.28 | 1.57 | 0.44 | −0.31 | 0.80 | 0.34 | −0.76 | −1.48 |
| −0.78 | −1.41 | 0.38 | −1.91 | −0.77 | 0.97 | −0.55 | −0.84 | −1.61 | 2.49 |
| −0.62 | 1.75 | −0.12 | −0.85 | 0.71 | −0.70 | 0.39 | −0.92 | 1.33 | 0.61 |

ABCD3

| | | | | | | | | | |
|---|---|---|---|---|---|---|---|---|---|
| −1.68 | −0.45 | 2.07 | −0.79 | −1.69 | −1.34 | 0.67 | 1.81 | 0.00 | 0.96 |
| 0.24 | 0.40 | 1.08 | 1.18 | 0.25 | 1.09 | 1.02 | −1.22 | 0.61 | 0.22 |
| 0.90 | 1.35 | 0.86 | 1.03 | 0.00 | 1.18 | 0.31 | 0.15 | 0.05 | −0.75 |
| 0.81 | 1.10 | −0.58 | −0.69 | −1.50 | −0.48 | 0.00 | 0.23 | −0.36 | −0.32 |
| −1.01 | 0.74 | 0.44 | 0.35 | −2.44 | −1.57 | 1.07 | 1.13 | 0.11 | −2.56 |
| −0.23 | −1.41 | −0.03 | −0.43 | −2.08 | 0.53 | 1.26 | −1.08 | 0.54 | −1.06 |

ABCD4

| | | | | | | | | | |
|---|---|---|---|---|---|---|---|---|---|
| −0.19 | −0.86 | 1.54 | 3.84 | 0.76 | 0.01 | −1.26 | −0.29 | 0.64 | −2.44 |
| 0.68 | −0.50 | −0.18 | −0.15 | 1.00 | −0.90 | −1.15 | 0.38 | 0.59 | −1.06 |
| −0.36 | 2.32 | 0.38 | 1.60 | 0.23 | 1.49 | −0.05 | −0.21 | −1.46 | −0.79 |
| 0.03 | −0.24 | −1.82 | −0.63 | −0.23 | 0.12 | −0.12 | 0.95 | −1.69 | −0.69 |
| 0.19 | −0.84 | −0.28 | 1.46 | 1.02 | −1.27 | −1.58 | 0.08 | −0.57 | 0.14 |
| −1.06 | 0.36 | 0.37 | −0.16 | 1.30 | 0.89 | −0.53 | 0.62 | 1.54 | −0.97 |

ABCE1

| | | | | | | | | | |
|---|---|---|---|---|---|---|---|---|---|
| −1.22 | 1.35 | 1.12 | −1.11 | −3.11 | −2.83 | 1.25 | 0.99 | 0.24 | 0.97 |
| 0.54 | 0.85 | 0.62 | 0.50 | 0.47 | 1.36 | 1.06 | 0.06 | 1.34 | 0.32 |
| 0.47 | 1.74 | −0.49 | 0.62 | 0.07 | 0.04 | −0.20 | −0.09 | 0.21 | 0.13 |
| 1.93 | 0.04 | 0.31 | 1.06 | −0.93 | 1.01 | 0.23 | −0.02 | −0.91 | −0.14 |
| −1.34 | −0.77 | −1.31 | 0.42 | −1.83 | −2.93 | −0.09 | −0.15 | 0.61 | −2.55 |
| −0.18 | −2.88 | 0.19 | −2.39 | −0.16 | 2.02 | 1.66 | 1.23 | 0.12 | 0.49 |

ABCF1

| | | | | | | | | | |
|---|---|---|---|---|---|---|---|---|---|
| −0.65 | 1.40 | 1.92 | −0.31 | −0.52 | −0.41 | 1.62 | 1.16 | −0.19 | −0.42 |
| 1.76 | 0.01 | 0.59 | 0.21 | −0.72 | −0.52 | −0.23 | −2.25 | −0.71 | 1.06 |
| 0.43 | 1.12 | −1.05 | −0.22 | −1.00 | −0.68 | −1.10 | 0.57 | −0.84 | −0.30 |
| 1.36 | 1.09 | 0.36 | −0.27 | −0.35 | −0.23 | −0.52 | 0.67 | −0.91 | −1.76 |
| −0.66 | −0.35 | 2.24 | 0.53 | −0.91 | −1.77 | −0.45 | 0.09 | 0.36 | −0.60 |
| −1.05 | −0.19 | 0.76 | −0.59 | 0.19 | 0.51 | 0.77 | 1.31 | −0.17 | 0.81 |

ABCF2

| | | | | | | | | | |
|---|---|---|---|---|---|---|---|---|---|
| −1.05 | 0.70 | 0.20 | −0.30 | −1.35 | −1.91 | 1.05 | 1.07 | −0.66 | 0.38 |
| −0.30 | −0.78 | −0.23 | 0.68 | 0.14 | 1.14 | 0.50 | −0.98 | 0.07 | 0.94 |
| 0.48 | 0.79 | −0.16 | −0.04 | −1.07 | −1.61 | −0.48 | 0.11 | 1.02 | 0.78 |
| −0.12 | −2.55 | 0.45 | 1.14 | −0.65 | 0.53 | −0.25 | 0.95 | −0.57 | 1.02 |
| −0.09 | −1.20 | −0.87 | −0.02 | −0.08 | −1.35 | 0.71 | 1.00 | 0.76 | −0.26 |
| −0.25 | −2.18 | −0.07 | −0.42 | 1.49 | 1.77 | 0.66 | 0.87 | −0.62 | 1.08 |

ABCF3

| | | | | | | | | | |
|---|---|---|---|---|---|---|---|---|---|
| −0.63 | 0.56 | 1.02 | −0.52 | −1.05 | 0.37 | 0.51 | 0.99 | −0.73 | 0.50 |
| 1.01 | 0.37 | 0.61 | 0.73 | −0.66 | 0.79 | 0.27 | −0.44 | −0.03 | 0.58 |
| −0.13 | 1.08 | −0.20 | 0.76 | −1.12 | −0.58 | −0.58 | 0.15 | −0.10 | −0.24 |
| 1.08 | 1.02 | 0.06 | 1.06 | −1.74 | 0.03 | −0.38 | −0.09 | −0.33 | 0.47 |
| −0.82 | 0.07 | 0.46 | 0.40 | −0.82 | −0.82 | −0.51 | 0.30 | 0.18 | −0.07 |
| −0.72 | 0.12 | 0.02 | −0.89 | −0.16 | 0.15 | −0.50 | −0.19 | −1.20 | 0.50 |

ABCG1

| | | | | | | | | | |
|---|---|---|---|---|---|---|---|---|---|
| 3.37 | −1.46 | 0.47 | 0.95 | −2.39 | 2.03 | −0.68 | −1.56 | 0.70 | 1.15 |
| 0.17 | 1.56 | 0.12 | −1.32 | −1.77 | −1.60 | 2.69 | −2.29 | 0.52 | −0.23 |
| 2.79 | −0.73 | −1.94 | −0.49 | −1.94 | −2.14 | −3.04 | −1.94 | 0.73 | −2.29 |
| −0.87 | −0.44 | −2.29 | −1.05 | 2.25 | −0.38 | 2.32 | 1.31 | −0.39 | 0.99 |
| −2.84 | −3.18 | −1.68 | −0.42 | 1.48 | −0.06 | 0.69 | 2.71 | 1.68 | 4.30 |
| 0.41 | 0.06 | −0.56 | −3.18 | −1.94 | 6.16 | −0.12 | 0.31 | 2.39 | 2.92 |

ABCG2

| | | | | | | | | | |
|---|---|---|---|---|---|---|---|---|---|
| 1.31 | −2.78 | 0.59 | 2.79 | 2.41 | −0.14 | −0.03 | 0.52 | 1.79 | 4.32 |
| −1.84 | −1.80 | 1.60 | −1.90 | −0.71 | −1.83 | 3.63 | 4.95 | −4.38 | 5.24 |
| 1.41 | −1.05 | −0.67 | −1.80 | −1.50 | −1.11 | −2.20 | −1.27 | −1.57 | −0.92 |
| 2.09 | −0.72 | −1.82 | −1.33 | 2.22 | 0.30 | −4.75 | −2.49 | 0.17 | 1.17 |
| 0.02 | 0.47 | 1.16 | 1.53 | 1.60 | −0.02 | −3.33 | 1.00 | 3.38 | 3.83 |
| 3.13 | −2.71 | 3.44 | −4.31 | −2.95 | −3.85 | −4.74 | −1.85 | −0.44 | 6.75 |

ABCG4

| | | | | | | | | | |
|---|---|---|---|---|---|---|---|---|---|
| −2.38 | −1.86 | 1.61 | 1.82 | 1.54 | 0.99 | 1.37 | 0.60 | 3.92 | −0.04 |
| −1.56 | 1.44 | −2.01 | −0.50 | 0.74 | −1.29 | −1.10 | 1.42 | −0.68 | −2.48 |
| −1.57 | 1.28 | 0.95 | −0.35 | 0.24 | 0.44 | −1.33 | −2.16 | 0.16 | 0.38 |
| −1.62 | −1.59 | −3.13 | −2.13 | 1.15 | −0.24 | −2.46 | 0.11 | 0.83 | 0.57 |
| −0.29 | 2.88 | 1.57 | −2.49 | −0.20 | 1.41 | −0.43 | 0.17 | 1.40 | −0.65 |
| −2.66 | 1.48 | −2.08 | 1.31 | 2.17 | 0.21 | −0.17 | 0.87 | 3.33 | 1.11 |

ABCG5

| | | | | | | | | | |
|---|---|---|---|---|---|---|---|---|---|
| 0.05 | −0.49 | 0.06 | −0.68 | −0.05 | −0.75 | −0.19 | −0.64 | −0.18 | −0.34 |
| −0.04 | 0.71 | −0.52 | −0.15 | −0.36 | −0.33 | −0.10 | 0.79 | 0.22 | −0.30 |
| −0.08 | 0.56 | 0.84 | 0.22 | 0.61 | 0.74 | 0.04 | −0.03 | 0.36 | 0.36 |
| −0.09 | −1.30 | −0.96 | 1.89 | 0.90 | −1.47 | −0.39 | 1.23 | 1.41 | 2.27 |
| −0.73 | 1.07 | 1.62 | 1.23 | 0.82 | 1.17 | −0.75 | −0.45 | −0.95 | −1.43 |
| −0.50 | −0.84 | 0.04 | −1.30 | −0.20 | −0.68 | −0.47 | −1.10 | 0.10 | −0.45 |

ABCG8

| | | | | | | | | | |
|---|---|---|---|---|---|---|---|---|---|
| 0.81 | 3.18 | 0.27 | 1.60 | 3.62 | 1.46 | 0.89 | −3.10 | −1.08 | −3.25 |
| 1.23 | −3.25 | −3.42 | −3.25 | −3.05 | 0.50 | −3.25 | 1.76 | 0.13 | 1.59 |
| 2.21 | 2.15 | 1.65 | −0.86 | 11.57 | 0.69 | 3.03 | −0.22 | 0.35 | 0.65 |
| −0.27 | −4.41 | 1.53 | −2.33 | 2.87 | −2.00 | −2.01 | 0.29 | 0.72 | 3.08 |
| 0.04 | −2.19 | 3.83 | −2.98 | 2.63 | −1.35 | −1.91 | −2.38 | 2.54 | −2.46 |
| −3.00 | −1.22 | −1.85 | 0.21 | 2.55 | 0.01 | −2.91 | −0.47 | 1.72 | −2.91 |

A clustered image map (“heat map”) as described by Weinstein et al. (1997, Science 275:343-349), which offers a visual summary of the patterns of ABC transporter expression across the 60 cell lines, is shown in FIG. 1. Table 3 shows the same data in numerical form.

TABLE 3

| Gene | A1 | B1 | C1 | D1 | E1 | F1 | G1 | H1 | I1 | J1 | K1 |
|---|---|---|---|---|---|---|---|---|---|---|---|
| ABCA1 | −2.07 | 0.38 | 0.36 | 0.27 | −0.11 | −2.50 | 0.37 | −0.50 | −1.83 | −0.24 | 0.57 |
| ABCA2 | 0.80 | 0.47 | −0.14 | 0.17 | 0.90 | 0.61 | −0.67 | 0.06 | −0.18 | 0.06 | −0.71 |
| ABCA3 | 3.79 | 2.40 | 0.93 | −4.96 | −4.64 | 5.11 | 3.78 | −2.88 | 4.64 | −0.63 | −8.09 |
| ABCA4 | 3.95 | −0.76 | −0.42 | −1.76 | −1.64 | 3.66 | −1.13 | −0.38 | 2.29 | −2.23 | −2.42 |
| ABCA5 | 0.26 | 0.90 | 0.80 | 0.35 | −0.43 | 1.38 | 1.71 | 0.44 | 0.55 | −0.60 | 0.57 |
| ABCA6 | −1.79 | 0.73 | −1.25 | −1.70 | −1.50 | −1.59 | 2.00 | 3.01 | −1.45 | −1.07 | −1.59 |
| ABCA7 | −0.83 | 0.92 | 0.90 | −0.89 | −0.02 | 0.43 | −0.24 | −1.65 | −1.10 | 0.47 | −0.58 |
| ABCA8 | 0.68 | −3.08 | −3.89 | 0.31 | 1.55 | −0.36 | −1.11 | −1.33 | 0.13 | 0.27 | −2.40 |
| ABCA9 | −1.61 | −1.52 | −1.07 | 7.23 | 5.52 | −1.41 | −1.52 | 4.54 | −1.27 | −0.89 | −1.41 |
| ABCA10 | 0.21 | 0.53 | 1.56 | −1.85 | −1.78 | 1.80 | 0.37 | −0.65 | −1.06 | −1.89 | −0.05 |
| ABCA12 | 5.42 | −2.52 | −2.07 | −2.52 | −2.32 | 7.13 | −2.52 | −2.97 | −2.27 | 0.67 | 0.54 |
| ABCA13 | −0.01 | −0.83 | −0.82 | 0.48 | NA | 0.30 | −0.75 | −0.97 | −0.91 | 0.64 | 1.24 |
| ABCB1 | −2.30 | 12.28 | −1.70 | −1.26 | −1.14 | −1.87 | −0.62 | 0.13 | −1.94 | 2.47 | −1.91 |
| ABCB2 | −0.84 | 0.07 | −0.01 | −1.90 | −1.38 | −2.83 | 1.82 | 1.78 | 1.70 | −1.72 | 3.03 |
| ABCB3 | −1.23 | −0.42 | 1.15 | −0.33 | 1.22 | −1.39 | 0.38 | 0.34 | −0.66 | 1.78 | 0.69 |
| ABCB4 | 4.34 | 7.61 | −2.46 | −0.45 | −0.93 | 1.02 | −2.73 | −3.72 | −2.05 | 5.66 | 1.00 |
| ABCB5 | −0.50 | −1.04 | −0.34 | 5.35 | 2.28 | −1.19 | −0.59 | −1.71 | −0.27 | −0.17 | −0.38 |
| ABCB6 | −0.02 | −0.29 | −0.19 | 0.55 | 0.28 | 0.37 | −0.26 | 1.37 | 0.22 | 1.14 | 1.52 |
| ABCB7 | −2.26 | 0.16 | 0.43 | −0.86 | −1.63 | −2.25 | 0.45 | 0.00 | −0.51 | −0.96 | 0.81 |
| ABCB8 | −1.52 | 0.77 | −0.06 | 0.13 | −0.75 | −1.85 | 0.36 | 0.96 | 1.15 | 0.56 | 1.97 |
| ABCB9 | 0.27 | 1.68 | 0.81 | 0.52 | 0.83 | −0.24 | −0.58 | −3.86 | −0.98 | 1.18 | 2.09 |
| ABCB10 | −1.66 | −0.37 | 1.09 | −0.79 | −0.75 | 0.21 | 0.41 | 0.36 | 0.46 | −0.34 | −0.83 |
| ABCB11 | 0.61 | −0.85 | −1.12 | −0.68 | −0.56 | −1.29 | −0.04 | 0.71 | −1.36 | −1.14 | 3.01 |
| ABCC1 | −0.78 | 0.18 | −0.25 | −0.95 | −1.03 | −2.48 | −0.60 | −0.21 | 0.05 | −0.51 | 1.70 |
| ABCC2 | −1.12 | −2.61 | −2.84 | 4.47 | 4.49 | −4.46 | 0.15 | −0.01 | −2.03 | 0.05 | −0.29 |
| ABCC3 | −5.06 | −4.83 | 1.41 | −2.84 | −1.27 | 0.18 | −0.36 | 1.67 | −3.52 | 1.03 | 1.42 |
| ABCC4 | −2.77 | −2.40 | 0.80 | 1.20 | 0.43 | −13.62 | 1.70 | −0.63 | −0.79 | 0.69 | 1.03 |
| ABCC5 | 2.44 | 1.14 | −2.67 | −1.67 | −1.54 | −1.60 | 0.46 | 2.43 | −5.36 | −4.85 | 2.08 |
| ABCC6 | 0.91 | 1.34 | 1.73 | −0.16 | 0.56 | 3.51 | −0.78 | −0.03 | 0.84 | −0.50 | 2.29 |
| ABCC7 | −1.37 | −4.36 | 2.07 | −0.02 | 1.20 | −1.65 | −0.43 | −0.15 | 1.78 | 2.22 | 0.19 |
| ABCC8 | 2.18 | −1.05 | −0.60 | −1.05 | −0.85 | −0.94 | −1.05 | −1.50 | 5.98 | −0.42 | −0.94 |
| ABCC9 | 0.48 | −2.02 | −2.27 | −1.32 | 2.03 | 1.22 | −2.61 | 2.82 | 1.67 | −0.61 | −2.83 |
| ABCC10 | 0.00 | 3.20 | −1.23 | −0.94 | 0.37 | −0.73 | 0.70 | 1.58 | 0.11 | 0.38 | 0.21 |
| ABCC11 | 1.77 | −0.14 | −2.30 | −1.85 | −1.73 | 16.67 | −1.21 | −0.46 | −0.09 | −2.32 | −2.51 |
| ABCC12 | 1.15 | −1.45 | 0.12 | −0.66 | 0.70 | 1.64 | −0.30 | −1.34 | 0.81 | 0.39 | −1.05 |
| ABCD1 | −0.26 | −3.96 | 2.72 | 4.12 | 4.55 | 1.29 | −0.47 | 1.44 | 1.33 | 0.19 | 1.63 |
| ABCD2 | 0.97 | −0.39 | −0.88 | −0.55 | 0.87 | 0.54 | −0.07 | −1.21 | 0.56 | 0.09 | −0.26 |
| ABCD3 | −1.68 | −0.45 | 2.07 | −0.79 | −1.69 | −1.34 | 0.67 | 1.81 | 0.00 | 0.96 | 0.24 |
| ABCD4 | −0.19 | −0.86 | 1.54 | 3.84 | 0.76 | 0.01 | −1.26 | −0.29 | 0.64 | −2.44 | 0.68 |
| ABCE1 | −1.22 | 1.35 | 1.12 | −1.11 | −3.11 | −2.83 | 1.25 | 0.99 | 0.24 | 0.97 | 0.54 |
| ABCF1 | −0.65 | 1.40 | 1.92 | −0.31 | −0.52 | −0.41 | 1.62 | 1.16 | −0.19 | −0.42 | 1.76 |
| ABCF2 | −1.05 | 0.70 | 0.20 | −0.30 | −1.35 | −1.91 | 1.05 | 1.07 | −0.66 | 0.38 | −0.30 |
| ABCF3 | −0.63 | 0.56 | 1.02 | −0.52 | −1.05 | 0.37 | 0.51 | 0.99 | −0.73 | 0.50 | 1.01 |
| ABCG1 | 3.37 | −1.46 | 0.47 | 0.95 | −2.39 | 2.03 | −0.68 | −1.56 | 0.70 | 1.15 | 0.17 |
| ABCG2 | 1.31 | −2.78 | 0.59 | 2.79 | 2.41 | −0.14 | −0.03 | 0.52 | 1.79 | 4.32 | −1.84 |
| ABCG4 | −2.38 | −1.86 | 1.61 | 1.82 | 1.54 | 0.99 | 1.37 | 0.60 | 3.92 | −0.04 | −1.56 |
| ABCG5 | 0.05 | −0.49 | 0.06 | −0.68 | −0.05 | −0.75 | −0.19 | −0.64 | −0.18 | −0.34 | −0.04 |
| ABCG8 | 0.81 | 3.18 | 0.27 | 1.60 | 3.62 | 1.46 | 0.89 | −3.10 | −1.08 | −3.25 | 1.23 |
| Gene | L1 | M1 | N1 | O1 | P1 | Q1 | R1 | S1 | T1 | U1 | V1 |
| ABCA1 | −0.48 | 0.76 | −2.19 | −2.40 | 1.41 | 2.97 | −2.64 | 0.16 | −2.62 | −2.58 | 2.60 |
| ABCA2 | −1.08 | 0.82 | −1.34 | −0.21 | −0.53 | 0.18 | 0.90 | −1.04 | −0.42 | −1.83 | 0.31 |
| ABCA3 | 1.68 | −2.88 | −1.35 | −0.42 | 2.30 | −4.62 | 3.42 | −0.65 | −2.75 | −7.00 | −1.56 |
| ABCA4 | −3.07 | −0.02 | 4.33 | 1.03 | −2.21 | −0.27 | −2.66 | −1.71 | −2.64 | −2.46 | 3.41 |
| ABCA5 | −0.28 | −0.40 | 0.87 | −0.21 | 0.87 | −1.98 | −0.78 | 1.69 | 0.21 | −0.72 | −0.59 |
| ABCA6 | −1.07 | −1.24 | −1.07 | −0.87 | −0.32 | −1.07 | −1.39 | −1.45 | −0.80 | −1.14 | −1.25 |
| ABCA7 | 1.22 | 0.41 | 1.34 | 1.30 | 0.97 | 0.91 | −0.58 | −1.02 | 3.36 | 0.29 | −0.39 |
| ABCA8 | −2.43 | −2.67 | −1.58 | −0.63 | −3.15 | −2.57 | 1.56 | −0.76 | −0.79 | −0.76 | −0.42 |
| ABCA9 | −0.89 | −1.06 | −0.89 | −0.69 | −0.14 | −0.89 | −1.21 | −1.27 | −0.62 | −0.96 | −1.07 |
| ABCA10 | −0.06 | −1.09 | −0.81 | 1.13 | 1.38 | 2.45 | 0.38 | 1.34 | 1.18 | −1.53 | −0.69 |
| ABCA12 | 1.72 | 2.40 | −1.17 | 1.77 | −1.14 | 1.58 | 3.23 | −2.27 | −1.62 | 6.90 | −2.07 |
| ABCA13 | 1.50 | 0.26 | 1.38 | −0.55 | −0.95 | −0.34 | −0.32 | −0.21 | −0.71 | −0.25 | 0.02 |
| ABCB1 | −1.98 | −3.05 | −2.09 | −2.39 | 11.08 | −1.86 | −1.54 | 3.19 | −2.13 | −1.95 | −2.64 |
| ABCB2 | −1.63 | 3.90 | −2.20 | 1.47 | −0.48 | 2.31 | −1.29 | −0.97 | 0.80 | 0.51 | −0.54 |
| ABCB3 | −1.11 | 1.59 | 3.72 | 0.16 | −0.13 | 0.89 | 1.23 | −0.70 | 0.41 | −1.00 | 0.39 |
| ABCB4 | 3.21 | −4.15 | 5.41 | −3.25 | 1.86 | 1.08 | −3.22 | 3.11 | −2.66 | −5.35 | −0.78 |
| ABCB5 | 1.01 | 0.40 | 0.04 | −0.22 | −0.76 | −0.76 | −0.52 | −0.07 | −0.48 | −0.48 | −1.16 |
| ABCB6 | 2.17 | 0.79 | 2.25 | −0.09 | −0.35 | −0.38 | −0.41 | 0.23 | 1.90 | −0.61 | −0.09 |
| ABCB7 | 0.25 | 1.07 | 1.05 | −0.12 | 1.27 | 1.09 | −0.07 | 2.61 | 0.87 | 1.71 | 1.77 |
| ABCB8 | 0.71 | 0.67 | 0.68 | −0.86 | −0.50 | −0.38 | −1.01 | −0.84 | 0.89 | 0.98 | −0.87 |
| ABCB9 | 0.92 | 1.07 | 1.63 | 0.43 | 0.60 | 0.70 | 0.05 | −0.51 | 1.11 | 1.02 | 1.56 |
| ABCB10 | 0.33 | 1.00 | −0.62 | −0.34 | 2.41 | 1.30 | 0.11 | 1.25 | 0.76 | 0.52 | −0.82 |
| ABCB11 | 4.82 | 2.76 | 3.01 | −1.81 | −1.12 | 4.79 | −1.57 | −2.25 | 2.71 | 1.28 | 4.05 |
| ABCC1 | −0.41 | 0.01 | 0.62 | 0.28 | −0.20 | 0.59 | 0.39 | 0.03 | −0.05 | 0.94 | 0.72 |
| ABCC2 | −3.25 | −1.00 | −1.19 | 4.85 | −4.11 | −5.64 | 0.53 | 2.70 | 4.69 | 3.18 | −1.31 |
| ABCC3 | 3.13 | 1.35 | 3.09 | 0.77 | −1.74 | 4.24 | 1.47 | −2.17 | 2.68 | 4.44 | −2.31 |
| ABCC4 | −1.52 | 0.21 | −0.83 | 0.43 | −0.39 | 0.21 | 0.72 | 0.23 | −1.83 | −0.12 | −0.50 |
| ABCC5 | −1.42 | 0.90 | −1.54 | 0.58 | 0.24 | −2.24 | −0.78 | −0.20 | 2.25 | −0.41 | 0.65 |
| ABCC6 | −2.72 | 1.79 | −2.25 | 0.33 | 3.83 | −2.74 | 0.74 | 1.13 | −2.29 | 3.88 | −2.79 |
| ABCC7 | 3.53 | −1.12 | 0.80 | −0.52 | −0.93 | 4.04 | 1.89 | 0.23 | 4.55 | 0.52 | −1.65 |
| ABCC8 | −0.42 | −0.59 | −0.42 | −0.22 | 0.33 | −0.42 | −0.74 | 2.11 | −0.15 | −0.49 | −0.60 |
| ABCC9 | −2.50 | −2.20 | −1.48 | 0.50 | −0.84 | −1.78 | 0.02 | −1.41 | −3.65 | −3.99 | −2.06 |
| ABCC10 | −0.39 | 0.11 | 1.22 | 4.44 | −0.99 | 0.03 | 0.51 | 0.39 | −4.39 | −3.17 | 0.35 |
| ABCC11 | 0.49 | 0.03 | −2.68 | 1.13 | −2.29 | −3.17 | 0.68 | 2.77 | −0.65 | 5.80 | 1.11 |
| ABCC12 | 1.24 | −0.28 | −1.18 | 0.83 | −0.81 | −0.63 | 1.86 | −0.44 | −0.25 | −1.23 | −0.23 |
| ABCD1 | 0.50 | 1.07 | 1.63 | −1.13 | −3.24 | −1.06 | −2.88 | −1.13 | −1.75 | −0.19 | −1.01 |
| ABCD2 | 0.78 | 0.34 | −0.84 | −0.40 | −0.59 | −0.24 | 1.42 | 0.01 | −1.70 | −0.57 | −0.14 |
| ABCD3 | 0.40 | 1.08 | 1.18 | 0.25 | 1.09 | 1.02 | −1.22 | 0.61 | 0.22 | 0.90 | 1.35 |
| ABCD4 | −0.50 | −0.18 | −0.15 | 1.00 | −0.90 | −1.15 | 0.38 | 0.59 | −1.06 | −0.36 | 2.32 |
| ABCE1 | 0.85 | 0.62 | 0.50 | 0.47 | 1.36 | 1.06 | 0.06 | 1.34 | 0.32 | 0.47 | 1.74 |
| ABCF1 | 0.01 | 0.59 | 0.21 | −0.72 | −0.52 | −0.23 | −2.25 | −0.71 | 1.06 | 0.43 | 1.12 |
| ABCF2 | −0.78 | −0.23 | 0.68 | 0.14 | 1.14 | 0.50 | −0.98 | 0.07 | 0.94 | 0.48 | 0.79 |
| ABCF3 | 0.37 | 0.61 | 0.73 | −0.66 | 0.79 | 0.27 | −0.44 | −0.03 | 0.58 | −0.13 | 1.08 |
| ABCG1 | 1.56 | 0.12 | −1.32 | −1.77 | −1.60 | 2.69 | −2.29 | 0.52 | −0.23 | 2.79 | −0.73 |
| ABCG2 | −1.80 | 1.60 | −1.90 | −0.71 | −1.83 | 3.63 | 4.95 | −4.38 | 5.24 | 1.41 | −1.05 |
| ABCG4 | 1.44 | −2.01 | −0.50 | 0.74 | −1.29 | −1.10 | 1.42 | −0.68 | −2.48 | −1.57 | 1.28 |
| ABCG5 | 0.71 | −0.52 | −0.15 | −0.36 | −0.33 | −0.10 | 0.79 | 0.22 | −0.30 | −0.08 | 0.56 |
| ABCG8 | −3.25 | −3.42 | −3.25 | −3.05 | 0.50 | −3.25 | 1.76 | 0.13 | 1.59 | 2.21 | 2.15 |

| Gene | W1 | X1 | Y1 | Z1 | A2 | B2 | C2 | D2 | E2 | F2 |
|---|---|---|---|---|---|---|---|---|---|---|
| ABCA1 | −1.91 | −0.66 | 0.39 | −0.51 | −0.08 | 1.23 | 1.26 | 0.04 | 3.28 | 1.64 |
| ABCA2 | 1.02 | 0.54 | 0.24 | 0.93 | −0.77 | 0.94 | −0.31 | −0.30 | 1.26 | 0.27 |
| ABCA3 | 0.82 | 3.35 | 4.11 | 3.42 | 3.31 | 4.15 | 2.43 | 3.05 | −1.55 | 2.51 |
| ABCA4 | 5.17 | −1.82 | 0.42 | 1.04 | 3.76 | −1.58 | −1.97 | −1.63 | −1.79 | 1.60 |
| ABCA5 | 0.01 | 1.21 | −0.06 | 0.09 | −0.34 | −0.81 | −0.08 | −2.13 | −3.41 | 0.01 |
| ABCA6 | −1.05 | −1.45 | −1.05 | −1.25 | 2.28 | −1.05 | −1.59 | −1.39 | −1.14 | −1.59 |
| AACA7 | −0.33 | 1.18 | −0.95 | −0.12 | −0.90 | 0.24 | 0.39 | −1.24 | 0.12 | 0.81 |
| ABCA8 | 1.53 | −1.59 | 0.91 | 0.79 | −0.29 | 0.48 | 0.24 | −0.86 | −3.24 | −0.97 |
| ABCA9 | −0.87 | −1.27 | −0.87 | −1.07 | 0.75 | −0.87 | −1.41 | −1.21 | −0.96 | −1.41 |
| ABCA10 | −0.76 | 1.27 | −0.34 | 1.87 | 0.55 | 0.35 | −0.27 | 1.21 | −0.68 | 0.86 |
| ABCA12 | −1.87 | −2.27 | −1.87 | −2.07 | −2.97 | −1.87 | 5.07 | 3.85 | 3.85 | 7.36 |
| ABCA13 | −0.91 | −0.18 | −0.47 | −0.24 | 0.20 | 0.70 | −0.55 | −1.05 | −0.70 | −0.52 |
| ABCB1 | −2.63 | −2.39 | 1.94 | −1.48 | 1.91 | −1.08 | 4.31 | 3.69 | 2.84 | −1.22 |
| ABCB2 | −0.38 | −1.15 | −0.44 | 0.59 | −0.10 | 1.46 | −2.78 | −0.89 | 1.33 | 1.37 |
| ABCB3 | 0.20 | 0.07 | −0.36 | −0.85 | −0.03 | 0.16 | 0.20 | −0.45 | 2.29 | −2.13 |
| ABCB4 | −2.03 | 1.84 | 3.16 | −5.94 | −4.52 | 0.01 | −0.31 | −0.73 | −2.76 | 2.04 |
| ABCB5 | −0.86 | −0.38 | −1.04 | −0.61 | −0.77 | −0.35 | 0.00 | −1.24 | −0.36 | −1.33 |
| ABCB6 | −0.36 | −0.02 | −0.86 | −1.73 | 0.87 | −0.19 | 1.09 | −0.90 | 0.58 | 0.40 |
| ABCB7 | −0.13 | −0.07 | −1.33 | −0.79 | −0.24 | −0.32 | −0.43 | −1.24 | 2.49 | 0.55 |
| ABCB8 | −1.12 | −1.05 | −3.00 | −1.09 | −1.84 | 1.39 | 0.70 | 1.14 | 0.26 | 2.11 |
| ABCB9 | −0.48 | 0.22 | −0.69 | −0.72 | −0.09 | −0.13 | −0.83 | 0.45 | −0.76 | 0.40 |
| ABCB10 | −1.10 | 0.33 | −1.48 | 0.54 | 0.22 | 0.01 | −0.45 | 0.25 | 1.40 | 0.52 |
| ABCB11 | −0.01 | −1.81 | −1.45 | −0.90 | 1.08 | −0.50 | −2.32 | −0.55 | −1.98 | −1.87 |
| ABCC1 | −1.08 | 0.01 | 1.09 | 1.91 | 0.56 | −0.08 | −0.84 | −0.13 | 0.18 | −1.63 |
| ABCC2 | −0.83 | −1.83 | −3.19 | −1.36 | −0.19 | −4.00 | 5.29 | −1.93 | −1.89 | −0.75 |
| ABCC3 | −2.36 | 3.30 | −3.61 | 3.35 | −1.81 | 4.02 | 1.82 | 2.86 | 2.73 | 0.85 |
| ABCC4 | 0.27 | 2.63 | 0.39 | 1.92 | 1.27 | −0.06 | 1.08 | 0.52 | −0.05 | 1.47 |
| ABCC5 | 0.56 | 0.44 | 0.89 | 0.13 | 2.51 | 1.96 | −4.19 | −1.08 | 0.38 | −0.30 |
| ABCC6 | −2.79 | −0.61 | 2.20 | −1.63 | −3.27 | 2.20 | −0.01 | 3.93 | 0.88 | 0.09 |
| ABCC7 | 3.65 | −0.90 | −0.28 | 0.29 | −0.43 | −0.46 | −0.81 | −1.77 | 0.64 | −1.46 |
| ABCC8 | −0.40 | 2.33 | −0.40 | 2.05 | 4.99 | −0.40 | −0.94 | −0.74 | −0.49 | −0.94 |
| ABCC9 | 1.37 | −0.51 | 1.23 | 1.47 | 1.05 | 0.87 | 0.80 | 5.09 | −3.99 | −0.13 |
| ABCC10 | 1.25 | −0.12 | 0.98 | 1.09 | −1.23 | 1.50 | −2.76 | 0.97 | 0.60 | 1.66 |
| ABCC11 | 2.91 | 1.13 | −2.62 | −2.07 | 1.27 | −1.67 | −0.34 | −1.72 | −1.07 | −1.00 |
| ABCC12 | 2.44 | 0.11 | 2.51 | 2.07 | 0.94 | 0.35 | 0.14 | 0.44 | −1.25 | −0.80 |
| ABCD1 | −0.05 | −0.97 | −2.00 | 0.41 | −1.69 | −1.95 | 0.26 | −2.35 | −1.66 | −0.78 |
| ABCD2 | 2.02 | 0.43 | 1.31 | 1.75 | −0.16 | 1.00 | −1.35 | −0.04 | −0.38 | −0.20 |
| ABCD3 | 0.86 | 1.03 | 0.00 | 1.18 | 0.31 | 0.15 | 0.05 | −0.75 | 0.81 | 1.10 |
| ABCD4 | 0.38 | 1.60 | 0.23 | 1.49 | −0.05 | −0.21 | −1.46 | −0.79 | 0.03 | −0.24 |
| ABCE1 | −0.49 | 0.62 | 0.07 | 0.04 | −0.20 | −0.09 | 0.21 | 0.13 | 1.93 | 0.04 |
| ABCF1 | −1.05 | −0.22 | −1.00 | −0.68 | −1.10 | 0.57 | −0.84 | −0.30 | 1.36 | 1.09 |
| ABCF2 | −0.16 | −0.04 | −1.07 | −1.61 | −0.48 | 0.11 | 1.02 | 0.78 | −0.12 | −2.55 |
| ABCF3 | −0.20 | 0.76 | −1.12 | −0.58 | −0.58 | 0.15 | −0.10 | −0.24 | 1.08 | 1.02 |
| ABCG1 | −1.94 | −0.49 | −1.94 | −2.14 | −3.04 | −1.94 | 0.73 | −2.29 | −0.87 | −0.44 |
| ABCG2 | −0.67 | −1.80 | −1.50 | −1.11 | −2.20 | −1.27 | −1.57 | −0.92 | 2.09 | −0.72 |
| ABCG4 | 0.95 | −0.35 | 0.24 | 0.44 | −1.33 | −2.16 | 0.16 | 0.38 | −1.62 | −1.59 |
| ABCG5 | 0.84 | 0.22 | 0.61 | 0.74 | 0.04 | −0.03 | 0.36 | 0.36 | −0.09 | −1.30 |
| ABCG8 | 1.65 | −0.86 | 11.57 | 0.69 | 3.03 | −0.22 | 0.35 | 0.65 | −0.27 | −4.41 |
| Gene | G2 | H2 | I2 | J2 | K2 | L2 | M2 | N2 | O2 | P2 |
| ABCA1 | 1.38 | 0.47 | 2.15 | −1.23 | 0.55 | −0.10 | −0.02 | 0.06 | 1.06 | 0.39 |
| ABCA2 | 1.11 | −0.07 | −0.24 | 1.45 | 0.76 | −0.04 | 0.02 | −0.23 | −0.70 | −0.49 |
| ABCA3 | −1.18 | 3.67 | 0.97 | 2.42 | 2.59 | −5.22 | −3.68 | −2.63 | −2.01 | −4.09 |
| ABCA4 | 1.31 | −3.02 | 0.32 | −1.11 | 3.96 | −2.10 | −2.05 | −1.64 | −2.16 | −1.71 |
| ABCA5 | −0.81 | −0.82 | −0.93 | 0.24 | 0.05 | −2.68 | 2.93 | 1.18 | 1.49 | 1.07 |
| ABCA6 | −1.39 | −0.15 | −1.25 | 0.17 | 0.17 | −0.35 | 5.91 | 2.65 | 2.78 | 3.38 |
| ABCA7 | −0.11 | −1.46 | 0.13 | 1.54 | −0.26 | 0.17 | 0.41 | −3.18 | −1.60 | 0.58 |
| ABCA8 | 0.45 | 0.77 | 2.21 | −0.05 | 1.15 | 0.50 | −1.27 | −1.32 | 5.88 | −2.12 |
| ABCA9 | −1.21 | 0.03 | −1.07 | 0.35 | 0.35 | −0.17 | 4.86 | 2.65 | 3.73 | 0.79 |
| ABCA10 | −0.59 | −0.52 | 0.18 | 0.98 | −1.35 | −1.64 | 1.01 | 0.70 | −1.63 | −1.09 |
| ABCA12 | 4.10 | 3.21 | −2.07 | −0.65 | −0.65 | −1.17 | −2.02 | −2.70 | −2.77 | 2.27 |
| ABCA13 | −0.44 | 0.20 | −0.79 | 1.70 | −0.50 | −0.75 | 0.71 | 0.75 | 0.76 | −0.22 |
| ABCB1 | 8.24 | 4.68 | −2.16 | −0.60 | −2.50 | −1.59 | 2.74 | −1.13 | 1.31 | −1.21 |
| ABCB2 | 0.87 | −1.27 | −0.77 | −0.63 | 0.54 | 3.54 | −1.29 | −0.48 | −0.21 | −3.38 |
| ABCB3 | −1.19 | 1.48 | 0.34 | −0.40 | −0.69 | 0.88 | −2.34 | −1.69 | −0.05 | −2.25 |
| ABCB4 | 0.51 | 4.59 | −3.56 | −0.48 | −3.08 | 0.07 | 4.98 | 5.07 | −0.25 | −1.73 |
| ABCB5 | −1.92 | −0.07 | 0.43 | −0.97 | −0.16 | −1.06 | −1.11 | 3.41 | 3.71 | 3.29 |
| ABCB6 | −1.35 | −0.78 | 0.00 | 0.15 | −0.75 | −0.34 | −0.23 | −1.13 | 1.57 | −0.13 |
| ABCB7 | −0.24 | −1.44 | −1.45 | −0.53 | −0.41 | 0.39 | −1.07 | −0.77 | −0.41 | −0.88 |
| ABCB8 | 2.97 | 1.25 | −1.04 | 0.56 | 0.33 | 0.19 | 0.21 | 0.85 | 0.32 | −0.15 |
| ABCB9 | 0.18 | 0.59 | 0.26 | 0.30 | −0.84 | −0.67 | 0.37 | 1.18 | 0.32 | −0.58 |
| ABCB10 | 0.31 | −5.22 | −0.71 | 0.07 | −0.40 | −0.58 | −0.70 | −0.75 | −0.18 | 0.05 |
| ABCB11 | −0.91 | −2.17 | −1.58 | −0.02 | 8.11 | 3.77 | −0.96 | −0.55 | −1.07 | −0.63 |
| ABCC1 | −0.23 | −0.07 | −0.22 | 0.65 | 0.06 | 0.17 | −1.31 | −0.96 | −1.14 | −0.31 |
| ABCC2 | −0.44 | −1.74 | −1.98 | 1.58 | −1.01 | −0.25 | 5.62 | 3.84 | 6.05 | 3.39 |
| ABCC3 | −0.06 | 2.19 | −0.66 | 2.85 | 1.00 | −1.16 | −3.13 | −2.72 | −3.24 | −0.27 |
| ABCC4 | −0.38 | 0.81 | 0.96 | 0.90 | −0.03 | 1.97 | −0.20 | −1.04 | 2.09 | 1.43 |
| ABCC5 | 0.07 | 0.73 | −2.74 | −0.18 | 1.32 | 3.53 | 0.40 | 0.74 | 0.91 | 0.56 |
| ABCC6 | 2.58 | 2.19 | 4.27 | −0.76 | −2.66 | −1.75 | −1.70 | −1.29 | −1.81 | 1.71 |
| ABCC7 | −1.17 | −2.73 | 0.85 | −1.77 | −2.87 | −0.68 | −0.06 | −0.36 | −1.98 | 0.90 |
| ABCC8 | −0.74 | 0.50 | −0.60 | 0.82 | 0.82 | 0.30 | −0.55 | −1.23 | −1.30 | −1.64 |
| ABCC9 | 1.79 | −3.01 | 4.47 | −0.25 | 0.61 | 0.80 | −2.85 | −0.85 | −1.03 | 7.59 |
| ABCC10 | 0.42 | 2.00 | −1.22 | −0.28 | 1.05 | 0.62 | −0.20 | −0.78 | −1.43 | −0.93 |
| ABCC11 | −1.79 | −2.22 | 0.53 | 0.02 | 0.45 | 2.51 | −0.15 | −1.14 | 1.94 | −1.80 |
| ABCC12 | −0.40 | 1.32 | 1.27 | −0.22 | 0.43 | −0.05 | −1.28 | −2.14 | −0.46 | −1.54 |
| ABCD1 | −2.02 | −2.04 | 0.47 | −1.83 | 0.91 | −1.27 | 1.51 | 3.17 | 0.58 | 4.25 |
| ABCD2 | −0.28 | 1.57 | 0.44 | −0.31 | 0.80 | 0.34 | −0.76 | −1.48 | −0.78 | −1.41 |
| ABCD3 | −0.58 | −0.69 | −1.50 | −0.48 | 0.00 | 0.23 | −0.36 | −0.32 | −1.01 | 0.74 |
| ABCD4 | −1.82 | −0.63 | −0.23 | 0.12 | −0.12 | 0.95 | −1.69 | −0.69 | 0.19 | −0.84 |
| ABCE1 | 0.31 | 1.06 | −0.93 | 1.01 | 0.23 | −0.02 | −0.91 | −0.14 | −1.34 | −0.77 |
| ABCF1 | 0.36 | −0.27 | −0.35 | −0.23 | −0.52 | 0.67 | −0.91 | −1.76 | −0.66 | −0.35 |
| ABCF2 | 0.45 | 1.14 | −0.65 | 0.53 | −0.25 | 0.95 | −0.57 | 1.02 | −0.09 | −1.20 |
| ABCF3 | 0.06 | 1.06 | −1.74 | 0.03 | −0.38 | −0.09 | −0.33 | 0.47 | −0.82 | 0.07 |
| ABCG1 | −2.29 | −1.05 | 2.25 | −0.38 | 2.32 | 1.31 | −0.39 | 0.99 | −2.84 | −3.18 |
| ABCG2 | −1.82 | −1.33 | 2.22 | 0.30 | −4.75 | −2.49 | 0.17 | 1.17 | 0.02 | 0.47 |
| ABCG4 | −3.13 | −2.13 | 1.15 | −0.24 | −2.46 | 0.11 | 0.83 | 0.57 | −0.29 | 2.88 |
| ABCG5 | −0.96 | 1.89 | 0.90 | −1.47 | −0.39 | 1.23 | 1.41 | 2.27 | −0.73 | 1.07 |
| ABCG8 | 1.53 | −2.33 | 2.87 | −2.00 | −2.01 | 0.29 | 0.72 | 3.08 | 0.04 | −2.19 |

| Gene | Q2 | R2 | S2 | T2 | U2 | V2 | W2 | X2 | Y2 |
|---|---|---|---|---|---|---|---|---|---|
| ABCA1 | −1.20 | −2.30 | 0.05 | 0.36 | −1.05 | 0.11 | −2.42 | 0.63 | 0.70 |
| ABCA2 | 0.03 | 0.26 | −1.04 | 0.92 | −0.08 | −0.72 | −1.99 | 1.06 | −0.41 |
| ABCA3 | −5.70 | −6.68 | −3.48 | 3.83 | 3.38 | −0.04 | 2.72 | −0.26 | 0.03 |
| ABCA4 | −1.11 | 2.83 | −1.67 | −1.59 | 1.10 | 1.84 | 3.97 | −0.16 | −2.72 |
| ABCA5 | −0.46 | 2.08 | −0.18 | −0.38 | −0.61 | −0.14 | 1.33 | −0.71 | 0.34 |
| ABCA6 | −0.78 | −0.80 | 5.69 | 0.83 | −2.04 | −0.20 | 4.21 | −0.28 | 2.11 |
| ABCA7 | −2.08 | −2.10 | −3.07 | 0.44 | 1.18 | 1.39 | −0.40 | 0.86 | −1.44 |
| ABCA8 | 1.11 | 1.46 | 3.73 | 1.91 | −1.05 | −1.74 | −1.39 | 2.97 | 5.63 |
| ABCA9 | 2.59 | 3.01 | −1.59 | 1.01 | −1.78 | −0.02 | −1.02 | −0.10 | −0.64 |
| ABCA10 | −2.17 | 0.48 | −0.55 | 0.74 | −0.51 | 0.50 | 0.63 | −1.41 | −0.60 |
| ABCA12 | −1.60 | −0.29 | 2.80 | 0.01 | 0.86 | 0.18 | −1.06 | −1.10 | −0.81 |
| ABCA13 | 0.15 | 0.41 | 0.45 | −0.66 | 0.70 | 0.78 | −0.01 | 0.73 | 0.38 |
| ABCB1 | −0.60 | −1.67 | −1.16 | −1.08 | 3.58 | −2.03 | −1.79 | −1.82 | 3.05 |
| ABCB2 | −0.49 | 0.92 | 0.52 | 1.79 | −0.95 | −1.89 | −2.32 | −0.70 | −0.41 |
| ABCB3 | 0.41 | −1.90 | 0.55 | 2.39 | −1.19 | −0.28 | −0.43 | 0.22 | −1.17 |
| ABCB4 | 10.01 | 3.18 | −0.49 | −2.76 | 0.83 | −0.31 | 3.23 | −5.78 | −0.69 |
| ABCB5 | 2.47 | 7.67 | 2.05 | 0.18 | −1.90 | −1.10 | −2.09 | −0.17 | −1.60 |
| ABCB6 | −0.47 | 0.65 | 0.39 | 1.54 | −0.70 | −0.88 | −1.16 | 0.21 | −0.18 |
| ABCB7 | −0.48 | −0.60 | −2.79 | −0.60 | 10.35 | 0.32 | −0.26 | −0.53 | 0.05 |
| ABCB8 | −0.11 | 0.26 | −2.46 | −2.96 | −0.16 | 0.65 | 0.37 | 1.63 | −0.96 |
| ABCB9 | 1.52 | −0.75 | 1.08 | 0.22 | −1.07 | 0.84 | 0.45 | −0.21 | −1.19 |
| ABCB10 | −1.37 | −0.08 | −0.59 | −1.09 | 1.16 | 1.70 | −0.53 | 0.28 | 0.97 |
| ABCB11 | −0.02 | −1.00 | −0.58 | −0.50 | −2.34 | −1.45 | −1.21 | −1.24 | 2.28 |
| ABCC1 | −1.60 | 0.03 | 0.38 | −0.56 | −0.04 | −0.44 | 0.24 | 1.35 | 0.41 |
| ABCC2 | 3.09 | 5.39 | 1.17 | 0.16 | −1.95 | −1.79 | 1.85 | 4.08 | 7.22 |
| ABCC3 | −2.19 | −3.25 | −2.74 | −0.40 | 3.39 | 1.35 | −3.38 | 3.95 | 1.92 |
| ABCC4 | −0.72 | 1.17 | 1.13 | 1.69 | 0.49 | −0.25 | −0.71 | −1.00 | −2.60 |
| ABCC5 | −0.57 | 4.42 | 3.96 | 0.42 | −4.74 | −2.33 | −1.38 | 0.51 | −0.45 |
| ABCC6 | −0.76 | −1.82 | −1.32 | 1.13 | 0.81 | 2.65 | −1.18 | −1.98 | −2.37 |
| ABCC7 | −1.72 | −4.59 | −0.47 | −0.43 | −2.04 | 0.72 | −2.84 | −1.73 | −2.90 |
| ABCC8 | −0.13 | −0.15 | −1.12 | 1.48 | −1.39 | 0.45 | 3.09 | 0.37 | 0.11 |
| ABCC9 | −3.63 | −3.65 | 1.15 | −2.02 | 0.31 | 0.06 | −0.40 | −3.13 | 8.23 |
| ABCC10 | 0.32 | −1.16 | −2.25 | 2.00 | 0.80 | −1.20 | −1.06 | 1.30 | −1.14 |
| ABCC11 | −1.19 | 0.62 | 1.19 | −1.67 | 4.78 | 0.24 | 1.19 | 2.32 | −0.69 |
| ABCC12 | −0.29 | −1.99 | 0.12 | 1.09 | −0.40 | −1.24 | −1.97 | 1.90 | −1.14 |
| ABCD1 | 2.33 | 2.26 | 1.10 | −0.49 | 0.10 | 2.37 | −0.19 | −1.48 | −2.21 |
| ABCD2 | 0.38 | −1.91 | −0.77 | 0.97 | −0.55 | −0.84 | −1.61 | 2.49 | −0.62 |
| ABCD3 | 0.44 | 0.35 | −2.44 | −1.57 | 1.07 | 1.13 | 0.11 | −2.56 | −0.23 |
| ABCD4 | −0.28 | 1.46 | 1.02 | −1.27 | −1.58 | 0.08 | −0.57 | 0.14 | −1.06 |
| ABCE1 | −1.31 | 0.42 | −1.83 | −2.93 | −0.09 | −0.15 | 0.61 | −2.55 | −0.18 |
| ABCF1 | 2.24 | 0.53 | −0.91 | −1.77 | −0.45 | 0.09 | 0.36 | −0.60 | −1.05 |
| ABCF2 | −0.87 | −0.02 | −0.08 | −1.35 | 0.71 | 1.00 | 0.76 | −0.26 | −0.25 |
| ABCF3 | 0.46 | 0.40 | −0.82 | −0.82 | −0.51 | 0.30 | 0.18 | −0.07 | −0.72 |
| ABCG1 | −1.68 | −0.42 | 1.48 | −0.06 | 0.69 | 2.71 | 1.68 | 4.30 | 0.41 |
| ABCG2 | 1.16 | 1.53 | 1.60 | −0.02 | −3.33 | 1.00 | 3.38 | 3.83 | 3.13 |
| ABCG4 | 1.57 | −2.49 | −0.20 | 1.41 | −0.43 | 0.17 | 1.40 | −0.65 | −2.66 |
| ABCG5 | 1.62 | 1.23 | 0.82 | 1.17 | −0.75 | −0.45 | −0.95 | −1.43 | −0.50 |
| ABCG8 | 3.83 | −2.98 | 2.63 | −1.35 | −1.91 | −2.38 | 2.54 | −2.46 | −3.00 |
| Gene | Z2 | A3 | B3 | C3 | D3 | E3 | F3 | G3 | H3 |
| ABCA1 | 0.92 | −0.68 | 2.06 | 0.62 | 2.04 | 2.95 | −1.93 | −0.63 | 0.71 |
| ABCA2 | 1.95 | −0.38 | 0.03 | −0.55 | −2.49 | 1.07 | −1.09 | −0.01 | 0.94 |
| ABCA3 | 5.19 | −0.71 | 2.84 | 6.30 | −5.10 | −2.39 | 5.23 | −7.28 | 0.04 |
| ABCA4 | −1.11 | −2.48 | 6.99 | 1.75 | 5.56 | −3.38 | −1.81 | 5.35 | −1.24 |
| ABCA5 | −0.07 | 0.67 | 0.55 | −1.92 | 1.05 | 0.62 | −2.31 | −0.12 | −0.59 |
| ABCA6 | 0.96 | 0.33 | 5.99 | −1.05 | 0.37 | −0.73 | 2.53 | −1.25 | −0.73 |
| ABCA7 | −0.35 | −0.40 | 0.85 | −0.36 | −1.24 | 0.30 | 3.56 | 0.43 | 1.75 |
| ABCA8 | 3.35 | 0.00 | −0.15 | 1.83 | −1.00 | −0.26 | 3.03 | 0.27 | 0.52 |
| ABCA9 | 1.13 | 0.51 | 4.89 | −0.87 | −1.52 | −0.55 | −1.52 | −1.07 | −0.55 |
| ABCA10 | 2.32 | −0.84 | 0.50 | −1.60 | 1.98 | 1.49 | −0.56 | 0.69 | −2.06 |
| ABCA12 | 0.13 | −0.49 | 2.77 | −1.87 | −2.52 | −1.55 | −2.52 | −2.07 | −1.55 |
| ABCA13 | −0.66 | −0.14 | −0.13 | 2.19 | 0.68 | −0.36 | −0.14 | −0.02 | −0.24 |
| ABCB1 | −0.60 | −1.97 | −2.46 | −1.88 | −1.74 | 0.53 | 0.01 | 1.92 | −0.73 |
| ABCB2 | −0.22 | 1.40 | 0.80 | 0.96 | 0.22 | 0.69 | −0.55 | 0.11 | 2.58 |
| ABCB3 | 0.32 | −0.60 | 0.35 | 1.89 | 1.68 | 0.34 | −1.32 | −1.09 | −0.33 |
| ABCB4 | −1.16 | 3.78 | −4.35 | −0.49 | 0.23 | −2.44 | 0.57 | −0.45 | −2.36 |
| ABCB5 | 0.45 | −0.73 | −1.08 | 1.00 | −1.23 | −0.75 | −0.77 | 1.12 | −0.54 |
| ABCB6 | 0.73 | −0.64 | −0.66 | −1.22 | −1.85 | −0.78 | 0.29 | 0.64 | −1.90 |
| ABCB7 | −2.18 | 0.03 | −2.10 | −1.15 | 0.50 | 2.07 | 0.02 | −0.18 | 0.98 |
| ABCB8 | −2.00 | 1.90 | −0.93 | −0.24 | 1.55 | −0.24 | 0.66 | −0.99 | −0.18 |
| ABCB9 | −1.55 | −0.21 | −0.43 | −1.19 | −1.17 | −2.77 | 0.15 | −1.77 | −0.76 |
| ABCB10 | −0.41 | 0.20 | −1.78 | −0.92 | −0.42 | 3.09 | 0.97 | 1.19 | 1.82 |
| ABCB11 | −0.02 | 0.92 | −1.88 | −1.29 | −1.16 | 2.42 | 1.16 | −1.60 | −0.15 |
| ABCC1 | 0.70 | 0.84 | 0.65 | −0.19 | 0.46 | 0.82 | 1.12 | 0.60 | 0.55 |
| ABCC2 | −4.28 | 0.94 | 0.27 | −2.97 | −1.03 | −1.41 | −4.27 | −0.49 | −5.62 |
| ABCC3 | −0.80 | 0.01 | 3.11 | −0.53 | −1.70 | −2.13 | −2.14 | −2.19 | −1.01 |
| ABCC4 | −0.70 | −0.40 | 1.10 | −0.14 | −0.99 | 1.74 | 1.07 | −0.31 | 1.25 |
| ABCC5 | 2.03 | 1.67 | −2.57 | −4.81 | 1.85 | 1.30 | 3.94 | 1.60 | −0.39 |
| ABCC6 | −0.76 | −2.13 | −1.86 | 0.00 | −1.90 | 3.52 | −1.46 | −0.08 | −0.89 |
| ABCC7 | 1.46 | −2.19 | −1.13 | 1.83 | 7.98 | 0.77 | 1.96 | 3.42 | 2.49 |
| ABCC8 | 1.60 | 0.98 | −1.64 | −0.40 | −1.05 | −0.08 | −1.05 | −0.60 | −0.08 |
| ABCC9 | −1.90 | 1.24 | 3.39 | 8.14 | −0.97 | −1.49 | 1.73 | 0.07 | 1.19 |
| ABCC10 | 0.17 | 0.28 | −0.28 | −0.91 | −0.46 | −0.08 | 0.00 | −0.73 | −0.53 |
| ABCC11 | −1.19 | 0.70 | −2.10 | −0.94 | −1.70 | 2.23 | −1.90 | −2.78 | −1.32 |
| ABCC12 | 2.14 | −0.23 | −0.43 | 0.98 | −0.98 | −0.44 | −1.75 | 1.24 | 0.60 |
| ABCD1 | 1.41 | −1.44 | 1.72 | 1.28 | −0.26 | 0.21 | −0.62 | −1.61 | −0.79 |
| ABCD2 | 1.75 | −0.12 | −0.85 | 0.71 | −0.70 | 0.39 | −0.92 | 1.33 | 0.61 |
| ABCD3 | −1.41 | −0.03 | −0.43 | −2.08 | 0.53 | 1.26 | −1.08 | 0.54 | −1.06 |
| ABCD4 | 0.36 | 0.37 | −0.16 | 1.30 | 0.89 | −0.53 | 0.62 | 1.54 | −0.97 |
| ABCE1 | −2.88 | 0.19 | −2.39 | −0.16 | 2.02 | 1.66 | 1.23 | 0.12 | 0.49 |
| ABCF1 | −0.19 | 0.76 | −0.59 | 0.19 | 0.51 | 0.77 | 1.31 | −0.17 | 0.81 |
| ABCF2 | −2.18 | −0.07 | −0.42 | 1.49 | 1.77 | 0.66 | 0.87 | −0.62 | 1.08 |
| ABCF3 | 0.12 | 0.02 | −0.89 | −0.16 | 0.15 | −0.50 | −0.19 | −1.20 | 0.50 |
| ABCG1 | 0.06 | −0.56 | −3.18 | −1.94 | 6.16 | −0.12 | 0.31 | 2.39 | 2.92 |
| ABCG2 | −2.71 | 3.44 | −4.31 | −2.95 | −3.85 | −4.74 | −1.85 | −0.44 | 6.75 |
| ABCG4 | 1.48 | −2.08 | 1.31 | 2.17 | 0.21 | −0.17 | 0.87 | 3.33 | 1.11 |
| ABCG5 | −0.84 | 0.04 | −1.30 | −0.20 | −0.68 | −0.47 | −1.10 | 0.10 | −0.45 |
| ABCG8 | −1.22 | −1.85 | 0.21 | 2.55 | 0.01 | −2.91 | −0.47 | 1.72 | −2.91 |

Legend for Table 3

| | |
|---|---|
| Legend | Gene |
| A1 | BR-MCF7 |
| B1 | UK-MCF7-ADR-RES |
| C1 | BR-MDA-MB-231-ATCC |
| D1 | ME-MDA-MB-435 |
| E1 | ME-MDA-N |
| F1 | BR-T-47D |
| G1 | BR-BT-549 |
| H1 | BR-HS578T |
| I1 | CNS-SF-268 |
| J1 | CNS-SF-295 |
| K1 | CNS-SF-539 |
| L1 | CNS-SNB-19 |
| M1 | CNS-SNB-75 |
| N1 | CNS-U251 |
| O1 | CO-HCT-116 |
| P1 | CO-HCT-15 |
| Q1 | CO-HT29 |
| R1 | CO-KM12 |
| S1 | CO-SW-620 |
| T1 | CO-HCC-2998 |
| U1 | CO-COLO205 |
| V1 | OV-OVCAR-3 |
| W1 | OV-OVCAR-4 |
| X1 | OV-OVCAR-5 |
| Y1 | OV-OVCAR-8 |
| Z1 | OV-SK-OV-3 |
| A2 | OV-IGROV1 |
| B2 | RE-TK-10 |
| C2 | RE-A498 |
| D2 | RE-ACHN |
| E2 | RE-786-0 |
| F2 | RE-RXF-393 |
| G2 | RE-CAKI-1 |
| H2 | RE-UO-31 |
| I2 | RE-SN12C |
| J2 | PR-DU-145 |
| K2 | PR-PC-3 |
| L2 | ME-LOXIMVI |
| M2 | ME-M14 |
| N2 | ME-MALME-3M |
| O2 | ME-SK-MEL-5 |
| P2 | ME-SK-MEL-28 |
| Q2 | ME-SK-MEL-2 |
| R2 | ME-UACC-257 |
| S2 | ME-UACC-62 |
| T2 | LC-A549-ATCC |
| U2 | LC-EKVX |
| V2 | LC-HOP-92 |
| W2 | LC-NCI-H23 |
| X2 | LC-NCI-H322M |
| Y2 | LC-NCI-H460 |
| Z2 | LC-NCI-H522 |
| A3 | LC-HOP-62 |
| B3 | LC-NCI-H226 |
| C3 | LE-SR |
| D3 | LE-MOLT-4 |
| E3 | LE-HL-60 |
| F3 | LE-K-562 |
| G3 | LE-CCRF-CEM |
| H3 | LE-RPMI-8226 |

Quantitative analysis shows that the pattern of expression is most characteristic of tissue of origin for melanoma (9 of the 10 melanoma cells cluster together on the dendrogram). The one melanoma line not found in the melanoma cluster (LOX-IMVI) is amelanotic and undifferentiated and has been shown to lack transcripts characteristic of melanoma (Stinson et al., 1992, Anticancer Res. 12:1035-1053). MDA-MB435 and MDA-N were originally thought to be from breast cancer, but their appearance within the melanoma cluster is consistent with strong molecular profile evidence that they are melanoma-derived or at least melanoma-like (Scherf et al., 2000, Nature Genet. 24:236-244; Ellison et al., 2002, Mol. Pathol. 55:294-299; Ross et al., 2000, Nature Genet. 24:227-235). MDA-N is an ERBB2 transfectant of MDA-MB435. CNS (5/6), renal (5/8), and ovarian (4/6) cells tend to form clusters, whereas the leukemia, colon, lung, breast and prostate cancer cell lines do not cluster well by tissue of origin. Overall, the coherence by tissue of origin is moderate (see Table 4 below), as indicated by a kappa statistic of 0.46, (with two-tailed 95% bootstrap confidence interval=0.33-0.60). The two lumenal, estrogen receptor-positive breast lines (T47D and MCF7) cluster together. Table 4 shows clusters observed after hierarchical agglomerative clustering of cell lines based on expression profiles, with average linkage algorithm and a distance metric of 1-r. The tree was cut at a level that produced 9 clusters, matching the number of tissue-of-origin cell line categories. The resulting kappa statistic, which reflects how well the clusters reflect tissue-of-origin, was 0.46, with a 95% two-tailed confidence interval of (+0.33 to +0.60).

TABLE 4
Hierarchical Agglomerative Clustering of Cell Lines
Based on ABC Gene Expression Profiles

| Cluster | Cell line |
|---|---|
| 1 | BR-HS578T |
| | LC-NCI-H460 |
| | LC-HOP-62 |
| | ME-MDA-N |
| | ME-MDA-MB-435 |
| | ME-MALME-3M |
| | ME-M14 |
| | ME-SK-MEL-2 |
| | ME-UACC-257 |
| | ME-SK-MEL-5 |
| | ME-SK-MEL-28 |
| | ME-UACC-62 |
| 2 | CNS-SF-268 |
| | LC-NCI-H522 |
| | LC-A549-ATCC |
| | LC-NCI-H226 |
| | LE-SR |
| | OV-SK-OV-3 |
| | OV-IGROV1 |
| | OV-OVCAR-4 |
| | OV-OVCAR-8 |
| | RE-SN12C |
| 3 | CO-HCT-15 |
| | RE-CAKI-1 LE-HL-60 |
| | RE-ACHN |
| | RE-UO-31 |
| | RE-A498 |
| | RE-RXF-393 |
| 4 | LE-MOLT-4 |
| | LE-CCRF-CEM |
| | ME-LOXIMVI |
| | OV-OVCAR-3 |
| | PR-PC-3 |
| 5 | BR-MDA-MB-231-ATCC |
| | CNS-SNB-75 |
| | CNS-SF-539 |
| | CNS-SNB-19 |
| | CNS-U251 |
| | CNS-SF-295 |
| | CO-HT29 |
| | CO-COLO205 |
| | CO-HCC-2998 |
| | LC-HOP-92 |
| | LE-RPMI-8226 |
| | RE-786-0 |
| 6 | BR-T-47D |
| | BR-MCF7 |
| | LC-NCI-H23 |
| 7 | BR-BT-549 |
| | LE-K-562 |
| | OV-OVCAR-5 |
| | PR-DU-145 |
| | RE-TK-10 |
| 8 | CO-SW-620 |
| | LC-EKVX |
| 9 | CO-HCT-116 |
| | CO-KM12 |
| | LC-NCI-H322M |

This database provides valuable information on the expression patterns of both known and currently uncharacterized ABC transporters. Some of the ABC transporters are expressed ubiquitously (e.g., ABCC1), whereas others are selectively expressed in particular cell types (e.g., ABCB5 in melanoma-derived cells; see inset in FIG. 1 (inset) and Table 5 below). Table 5 shows the genes that are statistically significantly associated with tissues of origin. B5, A9, D1, C2, and G5 are, on average, over-expressed in the melanomas, whereas A3, C3, and A7 are under-expressed in those cells. B6 is the only gene significantly over-expressed in the CNS cells, and C7 is the only gene over-expressed in the leukemia. Calculations are done for the 59 cell lines (excluding NCI/ADR-RES) using a Monte Carlo permutation t-test

TABLE 5
ABC Genes Statistically Significantly
Associated with Tissues of Origin

| | | Mean (± SD) in tissue | Adjusted |
|---|---|---|---|
| Significant | Tissue of | vs. mean (± SD) in the | permutation |
| gene | origin | rest | P value |
| B5 | Melanoma | 2.8 (± 2.6) | <0.0001 |
| | | vs. −0.6 (± 0.7) | |
| A9 | Melanoma | 2.9 (± 2.7) | <0.0001 |
| | | vs. −0.6 (± 1.3) | |
| D1 | Melanoma | 2.3 (± 1.8) | 0.0005 |
| | | vs. −0.4 (± 1.4) | |
| C2 | Melanoma | 3.7 (± 2.0) | 0.0014 |
| | | vs. −0.7 (± 2.8) | |
| A3 | Melanoma | −4.3 (± 1.4) | 0.0022 |
| | | vs. 0.8 (± 3.4) | |
| G5 | Melanoma | 0.8 (± 1.0) | 0.0215 |
| | | vs. −0.2 (± 0.7) | |
| C3 | Melanoma | −2.3 (± 1.0) | 0.0298 |
| | | vs. 0.6 (± 2.4) | |
| A7 | Melanoma | −1.2 (± 1.4) | 0.0467 |
| | | vs. 0.2 (± 1.1) | |
| B6 | CNS | 1.4 (± 0.8) | 0.0181 |
| | | vs. −0.1 (± 0.8) | |
| C7 | Leukemia | 3.1 (± 2.6) | 0.0239 |
| | | vs. −0.3 (± 1.9) | |

Langmann et al. (2003, Clin. Chem. 49:230-238) found high expression of ABCA2 in brain, ABCA3 in lung, and ABCB1 and ABCC4 in kidney. Data from the instant study with regard to the expression of these four genes is shown in Table 6 below.

TABLE 6
Association of Selected Genes with Tissue Types

| | | Sample 1 mean | |
|---|---|---|---|
| | | (± SD) vs. | |
| | Sample 1 (size) vs. | sample 2 mean | Permutation T- |
| Gene | sample 2 (size) | (± SD) | test P value |
| ABCA3 | Lung cancer: H522, | 4.1 (± 0.9) vs. | 0.0393 |
| | A549, EKVX (3) vs. rest | −0.3 (± 3.7) | |
| | (56) | | |
| ABCB1 | Renal (8) vs. else (51) | 2.4 (± 3.6) vs. | 0.0059 |
| | | −0.6 (± 2.4) | |
| ABCC4* | Renal (8) vs. else (51) | 0.5 (± 0.6) vs. | 0.3705 |
| | | −0.04 (± 2.3) | |
| | Melanoma (10) vs. else | 0.7 (± 1.1) vs. | 0.2164 |
| | (49) | −0.1 (± 2.3) | |
| | Breast (5) vs. else | −2.9 (± 6.2) vs. | 0.0161* |
| | (54) | 0.3 (± 1.1) | |
| | Prostate (2) vs. else | 0.4 (± 0.7) vs. | 0.6586 |
| | (57) | 0.03 (± 2.2) | |
| | CNS (6) vs. else (53) | −0.2 (± 1.0) vs. | 0.6734 |
| | | 0.1 (± 2.2) | |
| | Leukemia (6) vs. else | 0.4 (± 1.1) vs. | 0.4998 |
| | (53) | −0.004 (± 2.2) | |
| | Lung (9) vs. else (50) | −0.3 (± 1.3) vs. | 0.5603 |
| | | 0.1 (± 2.3) | |
| | Colon (7) vs. else (52) | −0.1 (± 0.8) vs. | 0.7918 |
| | | 0.1 (± 2.2) | |
| | Ovarian (6) vs. else | 1.0 (± 1.2) vs. | 0.1444 |
| | (53) | −0.1 (± 2.2) | |
| ABCA2 | Renal (8) vs. else (51) | 0.3 (± 0.7) vs. | 0.2364 |
| | | −0.06 (± 0.9) | |
| | Melanoma (10) vs. else | −0.1 (± 0.5) vs. | 0.6873 |
| | (49) | 0.01 (± 0.9) | |
| | Breast (5) vs. else | 0.1 (± 0.5) vs. | 0.7135 |
| | (54) | −0.02 (± 0.9) | |
| | Prostate (2) vs. else | 1.1 (± 0.5) vs. | 0.068 |
| | (57) | −0.04 (± 0.9) | |
| | CNS (6) vs. else (53) | −0.4 (± 0.8) vs. | 0.2448 |
| | | 0.03 (± 0.9) | |
| | Leukemia (6) vs. else | −0.4 (± 1.3) | 0.2972 |
| | (53) | vs. 0.03 (± 0.8) | |
| | Lung (9) vs. else (50) | 0.04 (± 1.1) | 0.8492 |
| | | vs. −0.01 (± 0.8) | |
| | Colon (7) vs. else (52) | −0.4 (± 0.9) vs. | 0.1879 |
| | | 0.04 (± 0.9) | |
| | Ovarian (6) vs. else | 0.4 (± 0.6) vs. | 0.2559 |
| | (53) | −0.05 (± 0.9) | |

*Based on the step down Bonferroni-Holm multiple comparison procedure, the adjusted P value is 0.1449.

When analyzed by Monte Carlo permutation t-test, the instant data show that ABCA2 is ubiquitously expressed throughout the 60 lines (p>0.61 for each of the nine tissues of origin), whereas ABCA3 is selectively expressed (p=0.039) in H522M, A549, and EKVX (all of them lung cancer lines). ABCB1 is indeed selectively expressed in the renal cancer cell lines (p=0.0059). However, ABCC4 is only moderately expressed in those cells (p>0.145 for each of the nine tissues of origin). This apparent discrepancy with respect to the results of Langman et al. may be due to heterogeneity of the human tissue samples used in that study or may reflect distinctive characteristics of the cancer cells. The distribution of ABC transporters on the gene dendrogram appears to be independent of sequence-homology categories. ABCB2 and ABCB3, known to function as heterodimeric components of the ER transport system for peptide antigen presentation, are found in different clusters, suggesting that their reported coordinate expression is disrupted in the cancer cells. Conversely, ABCG5 and ABCG8, which also form a heterodimer, show the expected concordance in expression pattern across the 60 cells (see FIG. 1).

Correlation of ABC Transporter mRNA Levels with Drug Resistance

In a previous study using cDNA microarrays, the 60 cell lines were found to cluster reasonably well by tissue of origin on the basis of expression patterns determined for a broad range of genes, but they did not cluster as well on the basis of patterns of drug sensitivity (Scherf et al., 2000, Nature Genet. 24:236-244). Furthermore, there was only a modest correspondence between the two clusterings. Hence, cell clusters in the instant study that appear similar for both ABC transporter expression and drug activity patterns are particularly interesting. Clusters such as that consisting of ACHN, UO-31, HCT15, and NCI-ADRRES fall into that category. ABCB1 (i.e., MDR1) is highly expressed in those cells.

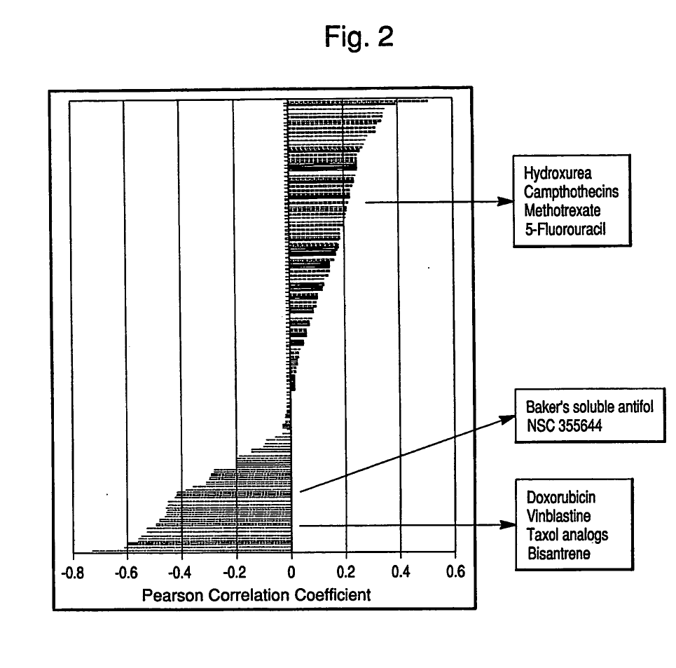

Since ABCB1 (MDR1-Pgp) extrudes molecules from the cell, the activity patterns of its substrates across the 60 cell lines are expected to be negatively correlated with its pattern of expression (Shoemaker et al, 2000, J. Natl. Cancer Inst. 92:4-5; Lee et al., 1994, Mol. Pharmacol. 46:627-638). FIG. 2 indicates that such is indeed the case for a set of 118 compounds with putatively known mechanisms of action (Weinstein et al., 1992, Science 275:343-349). Reported substrates (e.g., geldamycin, paclitaxel and its analogs, doxorubicin and vinblastine, and bisantrene) (Lee et al., 1994, Mol. Pharmacol. 46:627-638) indicated by blue bars show striking inverse correlations, whereas compounds not transported by MDR1 (e.g., hydroxyurea, camptothecins, methotrexate and 5-fluorouracil) are invariably found to be non-correlated or positively correlated (red bars). Of the 118 compounds, only two inversely correlated drugs, an anthrapyrazole-derivative (NSC 355644) and Baker's soluble antifol (NSC 139105), have not previously been established as MDR1 substrates (black bars). However, resistance to Baker's antifol is reversed by verapamil, a potent inhibitor of MDR1 transport, suggesting that it is indeed an MDR1 substrate. (Gupta et al., 1988, Br. J. Cancer 58:441-447).

To identify additional compounds that show significant inverse correlation with the expression of ABCB1, the analysis was extended to a larger data set containing the activity patterns of 1,429 compounds (Scherf et al., 2000, Nature Genet. 24:236-244). Pearson's correlation coefficients were calculated for a total of 67,163 relationships (47 genes X 1429 compounds) using bootstrap analysis with 10,000 iterations. The analysis yielded 130 highly inverse-correlated gene-drug pairs, shown in Table 7 below, sufficiently highly correlated in the negative sense that none of their 10,000 bootstrap samples were positively correlated.

TABLE 7
List of the 130 Drug-Gene Pairs Showing Significant
Inverse Correlation (p < 0.0001)

| GENE | DRUG | Correlation | Lower c.i. | Upper c.i. |
|---|---|---|---|---|
| ABCA1 | NSC 699479 | −0.4141 | −0.7128 | −0.0008 |
| ABCA1 | NSC 682066 | −0.3783 | −0.6859 | −0.0339 |
| ABCA1 | NSC 640085 | −0.3580 | −0.6365 | −0.0602 |
| ABCA1 | NSC 328426 | −0.2806 | −0.5720 | −0.0181 |
| ABCA2 | NSC 679265 | −0.3298 | −0.6697 | −0.0160 |
| ABCA3 | NSC 403170 | −0.4618 | −0.7453 | −0.0863 |
| ABCA3 | NSC 374979 | −0.4573 | −0.7756 | −0.0674 |
| ABCA3 | NSC 656178 | −0.4318 | −0.6978 | −0.0478 |
| ABCA3 | NSC 658142 | −0.4017 | −0.7008 | −0.0174 |
| ABCA3 | NSC 673187 | −0.3896 | −0.6805 | −0.0323 |
| ABCA3 | NSC 355256 | −0.3769 | −0.6525 | −0.0143 |
| ABCA3 | NSC 49842 | −0.3678 | −0.6572 | −0.0116 |
| ABCA4 | NSC 665925 | −0.4545 | −0.6904 | −0.1049 |
| ABCA4 | NSC 636092 | −0.3977 | −0.7207 | −0.0361 |
| ABCA4 | NSC 650771 | −0.3792 | −0.6526 | −0.0656 |
| ABCA4 | NSC 688235 | −0.3557 | −0.6502 | −0.0017 |
| ABCA9 | NSC 620480 | −0.4846 | −0.7670 | −0.1282 |
| ABCA9 | NSC 642915 | −0.4289 | −0.7093 | −0.0378 |
| ABCA12 | NSC 644751 | −0.5643 | −0.8096 | −0.2321 |
| ABCA12 | NSC 641240 | −0.5165 | −0.7764 | −0.1736 |
| ABCA12 | NSC 659853 | −0.5016 | −0.7615 | −0.1268 |
| ABCA12 | NSC 649666 | −0.3757 | −0.5964 | −0.0120 |
| ABCB1 | NSC 682066 | −0.7985 | −0.9289 | −0.2638 |
| ABCB1 | NSC 353076 | −0.7983 | −0.9580 | −0.1096 |
| ABCB1 | NSC 634791 | −0.7900 | −0.9350 | −0.0744 |
| ABCB1 | NSC 328426 | −0.7784 | −0.9348 | −0.1063 |
| ABCB1 | NSC 259968 | −0.7570 | −0.9430 | −0.0823 |
| ABCB1 | NSC 359449 | −0.7108 | −0.9282 | −0.0244 |
| ABCB1 | NSC 646946 | −0.7105 | −0.9172 | −0.0464 |
| ABCB1 | NSC 630678 | −0.7029 | −0.9140 | −0.0706 |
| ABCB1 | NSC 676864 | −0.6546 | −0.8785 | −0.1852 |
| ABCB1 | NSC 618757 | −0.6081 | −0.8443 | −0.0454 |
| ABCB1 | NSC 354975 | −0.6043 | −0.8747 | −0.0003 |
| ABCB1 | NSC 363997 | −0.5924 | −0.8488 | −0.1131 |
| ABCB1 | NSC694268 | −0.5914 | −0.8998 | −0.0464 |
| ABCB1 | NSC 374980 | −0.5590 | −0.8440 | −0.0009 |
| ABCB1 | NSC 636679 | −0.5530 | −0.7935 | −0.0198 |
| ABCB1 | NSC 652903 | −0.5303 | −0.8483 | −0.0861 |
| ABCB1 | NSC 156625 | −0.4657 | −0.7370 | −0.1379 |
| ABCB1 | NSC 651727 | −0.3910 | −0.6646 | −0.0152 |
| ABCB2 | NSC 25149 | −0.3794 | −0.6613 | −0.0173 |
| ABCB3 | NSC 622282 | −0.4406 | −0.7466 | −0.0188 |
| ABCB5 | NSC 670036 | −0.4561 | −0.6854 | −0.0912 |
| ABCB5 | NSC 671456 | −0.3650 | −0.6550 | −0.0733 |
| ABCB5 | NSC 280594 | −0.3477 | −0.6812 | −0.0483 |
| ABCB5 | NSC 693443 | −0.3300 | −0.6216 | −0.0044 |
| ABCB5 | NSC 694509 | −0.2924 | −0.6408 | −0.0202 |
| ABCB6 | NSC 277293 | −0.5055 | −0.8525 | −0.0118 |
| ABCB6 | NSC 92937 | −0.4335 | −0.7294 | −0.0003 |
| ABCB11 | NSC 284437 | −0.5273 | −0.7649 | −0.2016 |
| ABCB11 | NSC 150834 | −0.5267 | −0.8292 | −0.1390 |
| ABCB11 | NSC 15309 | −0.4683 | −0.8061 | −0.0041 |
| ABCB11 | NSC 326233 | −0.4430 | −0.7960 | −0.0823 |
| ABCB11 | NSC 695417 | −0.4270 | −0.7259 | −0.0648 |
| ABCB11 | NSC 335142 | −0.4214 | −0.7296 | −0.0326 |
| ABCC1 | NSC 617644 | −0.5087 | −0.7457 | −0.0606 |
| ABCC1 | NSC 208914 | −0.4950 | −0.7696 | −0.0858 |
| ABCC1 | NSC 670762 | −0.4326 | −0.7759 | −0.0297 |
| ABCC1 | NSC 641594 | −0.4324 | −0.7265 | −0.0232 |
| ABCC1 | NSC 666222 | −0.3675 | −0.6756 | −0.0149 |
| ABCC2 | NSC 639978 | −0.5210 | −0.7809 | −0.1074 |
| ABCC2 | NSC 638645 | −0.5028 | −0.7567 | −0.1350 |
| ABCC2 | NSC 637399 | −0.4969 | −0.8046 | −0.0475 |
| ABCC2 | NSC 639976 | −0.4670 | −0.7284 | −0.0584 |
| ABCC2 | NSC 641281 | −0.4621 | −0.7987 | −0.0440 |
| ABCC2 | NSC 674919 | −0.4608 | −0.7276 | −0.0426 |
| ABCC2 | NSC 687496 | −0.4544 | −0.7399 | −0.0505 |
| ABCC2 | NSC 693215 | −0.4377 | −0.7319 | −0.0225 |
| ABCC2 | NSC 639518 | −0.4350 | −0.7497 | −0.0105 |
| ABCC2 | NSC 684496 | −0.4340 | −0.8065 | −0.0366 |
| ABCC2 | NSC 634458 | −0.4326 | −0.7253 | −0.0429 |
| ABCC2 | NSC 618315 | −0.4247 | −0.6922 | −0.0282 |
| ABCC2 | NSC 696916 | −0.4224 | −0.6921 | −0.0913 |
| ABCC2 | NSC 692754 | −0.4016 | −0.7112 | −0.0572 |
| ABCC3 | NSC 641240 | −0.5829 | −0.8288 | −0.1961 |
| ABCC3 | NSC 644751 | −0.5748 | −0.8369 | −0.2455 |
| ABCC3 | NSC 641245 | −0.5702 | −0.8121 | −0.1912 |
| ABCC3 | NSC 658450 | −0.5526 | −0.7915 | −0.1342 |
| ABCC3 | NSC 639366 | −0.5003 | −0.7945 | −0.0301 |
| ABCC3 | NSC 641594 | −0.4994 | −0.7852 | −0.0903 |
| ABCC3 | NSC 658142 | −0.4982 | −0.7600 | −0.0877 |
| ABCC3 | NSC 627991 | −0.4741 | −0.7600 | −0.0846 |
| ABCC3 | NSC 267461 | −0.4000 | −0.7145 | −0.0148 |
| ABCC3 | NSC 641820 | −0.3991 | −0.7507 | −0.0134 |
| ABCC3 | NSC 670289 | −0.3824 | −0.7020 | −0.0123 |
| ABCC4 | NSC 251820 | −0.4340 | −0.7576 | −0.0104 |
| ABCC5 | NSC 155694 | −0.4494 | −0.8181 | −0.0507 |
| ABCC5 | NSC 352299 | −0.4318 | −0.7414 | −0.0500 |
| ABCC5 | NSC 604574 | −0.4123 | −0.8082 | −0.0222 |
| ABCC5 | NSC 21075 | −0.3650 | −0.6592 | −0.0430 |
| ABCC6 | NSC 269754 | −0.4649 | −0.7735 | −0.0643 |
| ABCC7 | NSC 86715 | −0.5696 | −0.8732 | −0.0762 |
| ABCC7 | NSC 178249 | −0.5603 | −0.8466 | −0.1454 |
| ABCC7 | NSC 654968 | −0.5519 | −0.8471 | −0.0705 |
| ABCC7 | NSC 627787 | −0.5471 | −0.8300 | −0.0358 |
| ABCC7 | NSC 626030 | −0.5025 | −0.7757 | −0.0001 |
| ABCC7 | NSC 6171 | −0.4552 | −0.7797 | −0.0628 |
| ABCC7 | NSC 670766 | −0.4378 | −0.7268 | −0.0151 |
| ABCC7 | NSC 695914 | −0.4297 | −0.6702 | −0.0109 |
| ABCC8 | NSC 626578 | −0.4335 | −0.7453 | −0.0497 |
| ABCC9 | NSC 352277 | −0.3094 | −0.6843 | −0.0083 |
| ABCC11 | NSC 671136 | −0.3994 | −0.6727 | −0.0141 |
| ABCD1 | NSC 73306 | −0.6029 | −0.8622 | −0.1067 |
| ABCD1 | NSC 69187 | −0.5711 | −0.8540 | −0.0643 |
| ABCD1 | NSC 338258 | −0.5453 | −0.8298 | −0.1420 |
| ABCD1 | NSC 143095 | −0.5337 | −0.7979 | −0.1363 |
| ABCD1 | NSC 645161 | −0.5134 | −0.7696 | −0.1293 |
| ABCD1 | NSC 692759 | −0.5034 | −0.7668 | −0.0311 |
| ABCD1 | NSC 692758 | −0.5012 | −0.8359 | −0.0152 |
| ABCD1 | NSC 645812 | −0.4825 | −0.7794 | −0.1247 |
| ABCD1 | NSC 645813 | −0.4824 | −0.7206 | −0.1001 |
| ABCD1 | NSC 640499 | −0.4694 | −0.7515 | −0.1096 |
| ABCD1 | NSC 692754 | −0.4680 | −0.7388 | −0.0963 |
| ABCD1 | NSC 71795 | −0.4642 | −0.7525 | −0.0318 |
| ABCD1 | NSC 627168 | −0.4599 | −0.7891 | −0.0708 |
| ABCD1 | NSC 685126 | −0.4464 | −0.7754 | −0.0074 |
| ABCD1 | NSC 71851 | −0.4425 | −0.7446 | −0.0091 |
| ABCD1 | NSC 645814 | −0.4346 | −0.6665 | −0.1002 |
| ABCD1 | NSC 163501 | −0.4301 | −0.6992 | −0.0232 |
| ABCD1 | NSC 645830 | −0.4258 | −0.7295 | −0.0566 |
| ABCD1 | NSC 653438 | −0.4252 | −0.7422 | −0.0441 |
| ABCD1 | NSC 687308 | −0.4236 | −0.7402 | −0.0268 |
| ABCD1 | NSC 126849 | −0.4214 | −0.7422 | −0.1153 |
| ABCD1 | NSC 670692 | −0.4001 | −0.7358 | −0.0057 |
| ABCD3 | NSC 19893 | −0.4232 | −0.7548 | −0.0024 |
| ABCD4 | NSC 106399 | −0.4232 | −0.7570 | −0.0212 |
| ABCF1 | NSC 163501 | −0.5274 | −0.7773 | −0.1227 |
| ABCG2 | NSC 668844 | −0.4615 | −0.7502 | −0.0604 |
| ABCG2 | NSC 694002 | −0.3627 | −0.6731 | −0.0375 |
| ABCG8 | NSC 209835 | −0.4443 | −0.7268 | −0.0208 |

The 18 compounds that were inversely correlated with ABCB1 expression and that survived this statistical screening share structural features (large size, polyaromatic backbone, amphipathic character) with the well-known MDR1 substrates (Rabow et al., 2002, J. Med. Chem. 45:818-840). NSC 328426 (phyllanthoside), NSC 259968 (Bouvardin), and NSC 156625 (Coralyne) have been tested in various laboratories and shown to interact with MDR1 (Lee et al., 1994, Mol. Pharmacol. 46:627-638; Gupta et al., 1988, Br. J. Cancer 58:441-447). The rest have not previously been implicated in MDR1-mediated resistance.

Evidence that Correlations Predict Drug Resistance Due to ABC Transporters

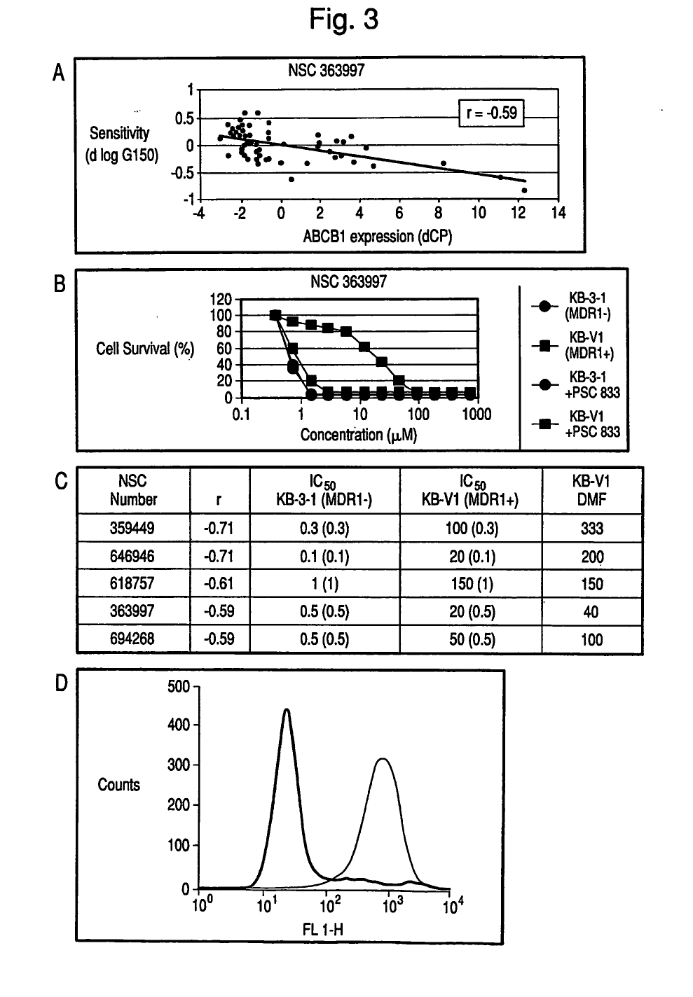

To test whether our approach using the NCI-60 does, in fact, identify new substrates, an MTT assay is used to test all top-scoring compounds that were available from DTP for follow-up experiments. KB-3-1, a human carcinoma cell line, and KB-V1, a multidrug resistant derivative of KB-3-1 that over-expresses MDR1-P-gp (Shen et al., 1986, J. Biol. Chem. 261:7762-7770), are used for the tests. FIG. 3 shows a typical result. In comparison with the parental line, KB-V1 cells are resistant to NSC 363997. PSC 833, a specific MDR1 antagonist, reverses the resistance, providing evidence that the resistance is linked to Pgp function. Further experiments show that KB-V1 cells are 30- to 300-fold less sensitive than KB-3-1 cells to all 6 compounds available for study, which are as follows: NSC 363997, NSC 359449, NSC 646946, NSC 618757, NSC 363997, NSC694268. This resistance of KB-V-1 cells is invariably reversible by PSC 833. The intrinsic fluorescence of one of the compounds, NSC 634791, allows for the measurement of the effect of MDR1 activity on its export from cells. Following incubation with NSC 634791 for 10 min at 37° C., MDR1-positive cells contain less of the fluorescent compound than the parental KB-3-1 cell line (FIG. 3). The decreased accumulation is completely reversible by addition of 2 μM PSC 833 (which had no effect on the parental cells), further corroborating the hypothesis that NSC 634791 is an MDR1 substrate.

In addition to the above described results for ABCB1, the results in Table 7 indicate that several ABC transporters, some of unknown function, can influence the response of cells to treatment. Assuming functional relationships, the compounds are predicted to be substrates of the respective ABC transporters. To verify this hypothesis, independent follow-up experiments were performed in defined systems for the most interesting correlative findings. The results of these experiments for two transporter drug pairs, one involving ABCC2 (MRP2) and the other involving ABCC11, are shown below.

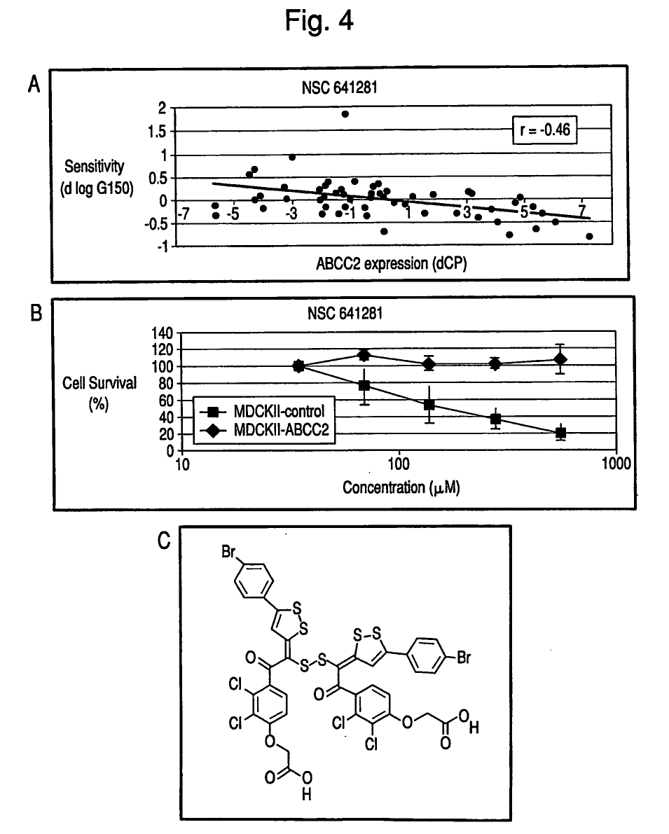

The ABCC (MRP) subfamily is comprised of nine members that transport structurally diverse lipophilic anions and function as drug efflux pumps (Kruh and Belinsky, 2003, Oncogene 22:7537-52). ABCC2-MRP2 is a canalicular efflux pump with a role in the hepatobiliary excretion of bilirubin glucuronide as well as numerous pharmaceuticals. Of the 1429 compounds analyzed in this study, 14 were shown by the stringent bootstrap criterion described above to be less active in ABCC2-overexpressing cells (Table 7). One of these compounds, NSC 641281 (shown in FIG. 4, Panel C), was available from DTP for further testing. To verify whether the highly significant correlation between the activity of NSC 641281 and ABCC2 expression implies a functional relationship in which ABCC2 protects the cells by exporting the compound, ABCC2-transfected MDCKII cells and control cells were compared in MTT assays (FIG. 4, Panel B). In sharp contrast to the control (sham-transfected) cells, the ABCC2-overexpressing MDCKII cells proved extremely resistant to NSC 641281, thus indicating that NSC 641281 is indeed an ABCC2-MRP2 substrate.

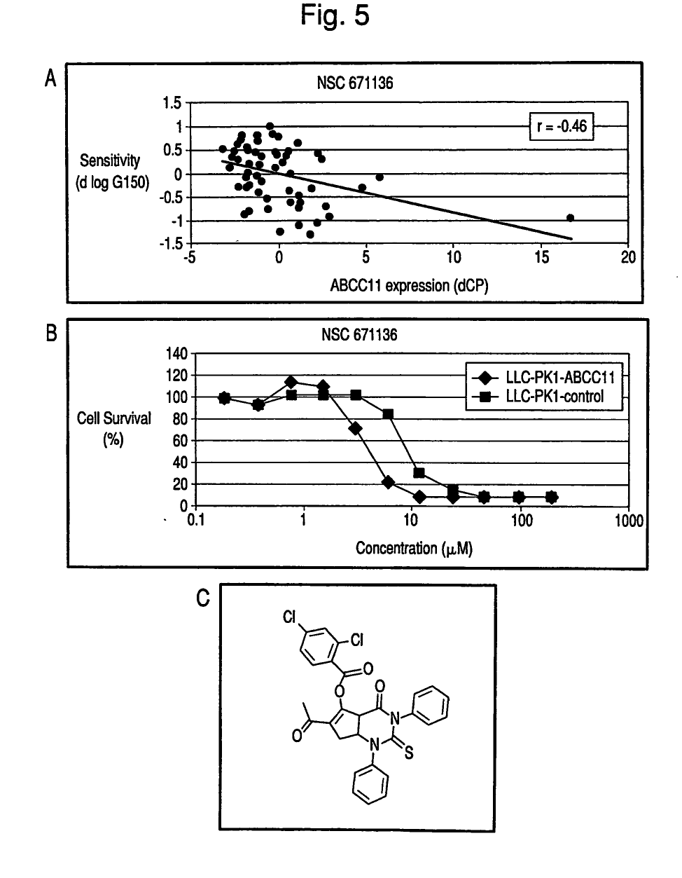

ABCC11, a recently identified member of the superfamily, has been shown to mediate the ATP-dependent transport of cyclic nucleotides and confer resistance to certain nucleotide analogs (Guo et al., 2003, J. Biol. Chem. 278:29509-29514). One compound, NSC 671136 (shown in FIG. 5, Panel C), met the stringent bootstrap criterion for significant inverse correlation with the expression of ABCC11 in the 60 cell lines (FIG. 5, Panel A). An MTT assay was used to assess whether over-expression of ABCC11 can confer resistance to the NSC 671136 compound. As shown in FIG. 5, Panel B, ABCC11-transfected LLC-PK1 cells were two- to three-fold more resistant to NSC 671136 than were control, sham-transfected cells. The correlation of gene expression with sensitivity thus identified a novel ABCC11 substrate, indicating that ABCC11-mediated resistance can extend to types of compounds other than nucleotide analogs.

### Positive Correlations Identify Compounds Potentiated by ABCB1

The positive correlation between activity and ABCB1 expression for some of the compounds, as shown in Table 8 below, suggests that those compounds can inhibit growth of the cancer cells more strongly if MDR1 is over-expressed.

TABLE 8
Compounds with an Antiproliferative Activity that is Positively Correlated
with ABCB1 Expression (From a Screen of 1430 Compounds)

| | | Pearson's Correlation | | | Pearson's Correlation |
|---|---|---|---|---|---|
| DRUG | GENE | Coeff | DRUG | GENE | Coeff |
| NSC697653 | B1 | 0.1718 | NSC693443 | B1 | 0.1842 |
| NSC676427 | B1 | 0.1843 | NSC106399 | B1 | 0.1871 |
| NSC688493 | B1 | 0.2012 | NSC681683 | B1 | 0.2079 |
| NSC691578 | B1 | 0.2102 | NSC696124 | B1 | 0.2104 |
| NSC163501 | B1 | 0.2122 | NSC696992 | B1 | 0.2171 |
| NSC640737 | B1 | 0.2176 | NSC600286 | B1 | 0.2177 |
| NSC656158 | B1 | 0.2218 | NSC657279 | B1 | 0.2253 |
| NSC268242 | B1 | 0.2268 | NSC697686 | B1 | 0.2297 |
| NSC113764 | B1 | 0.2318 | NSC645351 | B1 | 0.2327 |
| NSC368390 | B1 | 0.2331 | NSC100045 | B1 | 0.2342 |
| NSC126849 | B1 | 0.2357 | NSC56030 | B1 | 0.2372 |
| NSC375575 | B1 | 0.2400 | NSC694490 | B1 | 0.2438 |
| NSC694002 | B1 | 0.2442 | NSC638498 | B1 | 0.2464 |
| NSC8120 | B1 | 0.2468 | NSC95678 | B1 | 0.2475 |
| NSC26647 | B1 | 0.2476 | NSC281818 | B1 | 0.2476 |
| NSC671041 | B1 | 0.2610 | NSC697189 | B1 | 0.2621 |
| NSC641548 | B1 | 0.2632 | NSC281817 | B1 | 0.2646 |
| NSC605440 | B1 | 0.2658 | NSC174121 | B1 | 0.2662 |
| NSC679431 | B1 | 0.2672 | NSC632790 | B1 | 0.2677 |
| NSC271674 | B1 | 0.2678 | NSC300288 | B1 | 0.2734 |
| NSC284751 | B1 | 0.2759 | NSC143095 | B1 | 0.2763 |
| NSC693131 | B1 | 0.2788 | NSC134033 | B1 | 0.2800 |
| NSC693869 | B1 | 0.2805 | NSC102817 | B1 | 0.2813 |
| NSC652893 | B1 | 0.2821 | NSC694509 | B1 | 0.2823 |
| NSC330465 | B1 | 0.2872 | NSC163443 | B1 | 0.2891 |
| NSC633713 | B1 | 0.2908 | NSC600285 | B1 | 0.2935 |
| NSC100046 | B1 | 0.2949 | NSC693623 | B1 | 0.2952 |
| NSC184692 | B1 | 0.2971 | NSC302325 | B1 | 0.2979 |
| NSC602313 | B1 | 0.3012 | NSC698459 | B1 | 0.3095 |
| NSC319947 | B1 | 0.3098 | NSC382054 | B1 | 0.3151 |
| NSC132483 | B1 | 0.3151 | NSC693323 | B1 | 0.3180 |
| NSC382035 | B1 | 0.3180 | NSC646714 | B1 | 0.3262 |
| NSC382049 | B1 | 0.3279 | NSC382034 | B1 | 0.3302 |
| NSC32065 | B1 | 0.3336 | NSC645818 | B1 | 0.3377 |
| NSC689530 | B1 | 0.3398 | NSC298276 | B1 | 0.3398 |
| NSC689529 | B1 | 0.3465 | NSC267229 | B1 | 0.3505 |
| NSC697131 | B1 | 0.3514 | NSC697138 | B1 | 0.3551 |
| NSC176326 | B1 | 0.3552 | NSC285706 | B1 | 0.3654 |
| NSC694489 | B1 | 0.3742 | NSC697137 | B1 | 0.3828 |
| NSC382053 | B1 | 0.3841 | NSC142055 | B1 | 0.3935 |
| NSC697135 | B1 | 0.4052 | NSC692756 | B1 | 0.4093 |
| NSC692759 | B1 | 0.4149 | NSC697120 | B1 | 0.4167 |
| NSC691081 | B1 | 0.4305 | NSC51143 | B1 | 0.4328 |
| NSC697128 | B1 | 0.4568 | NSC697130 | B1 | 0.4582 |
| NSC692754 | B1 | 0.4616 | NSC692758 | B1 | 0.4825 |
| NSC697129 | B1 | 0.4854 | NSC73306 | B1 | 0.5389 |
| NSC697125 | B1 | 0.5604 | NSC693871 | B1 | 0.6160 |

For some transporters, including MDR1, several high positive correlations are much higher than would be expected from sampling variation. For the top 10 correlations, the minimum false discovery rate was 0.305. Thus the effects of at least some of the compounds increase systematically with higher MDR1 expression in the NCI-60.

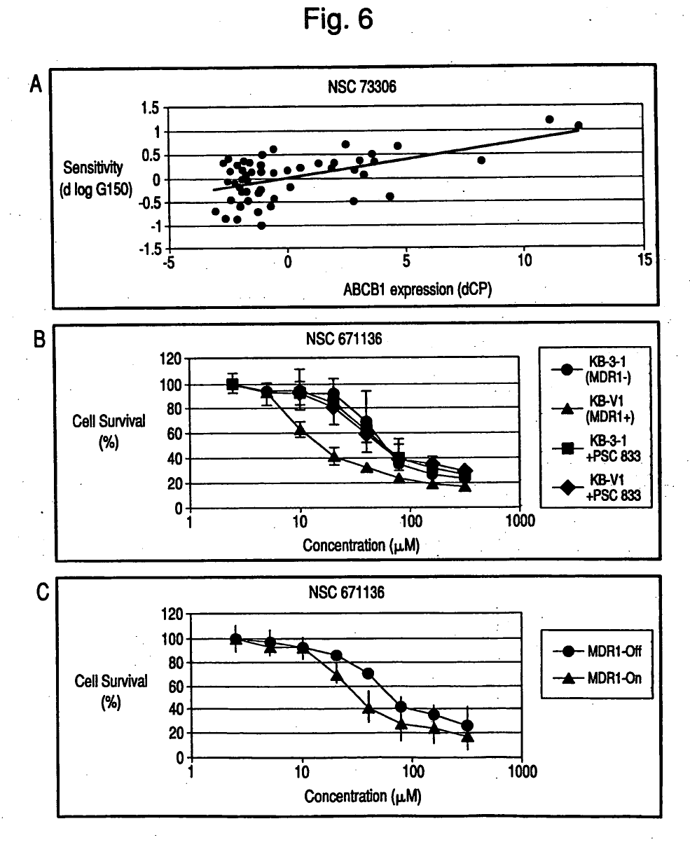

To confirm that compounds identified via the correlation analysis had an anti-proliferative activity that was potentiated by the ABCB1 transporter, the MTT assay using the KB-3-1/KB-V1 cell pair was employed to test the top-scoring compound that was available from DTP, NSC 73306. FIG. 6, Panel B shows that KB-V1 cells are four- to five-fold more sensitive than the parental KB-3-1. The finding that PSC 833 completely reversed sensitivity of KB-V1 cells to NSC 73306 (FIG. 6, Panel B) strongly suggests that the increased sensitivity is due to the function of MDR1, not to other, nonspecific properties of the KB-V1 cells.

Two other homologs of NSC 73306, NSC 73304 and NSC 73305, are also tested in the assay system described in the above paragraphs. Similar to the results obtained with NSC 73306, assays on these other two compounds show that KB-V1 cells are several-fold more sensitive than the parental KB-3-1 and that PSC 833 completely reverses sensitivity of KB-V1 cells to NSC 73304 and NSC 73305.

To substantiate further that the observed potentiation of NSC 73306 was not due to nonspecific factors arising during the generation of KB-V1, MTT assays are repeated using HeLa-transfectants in which human MDR1 is under tetracycline control. In these cells, addition of tetracycline suppresses transcription of MDR1 mRNA, and, over a period of a few days, MDR1 disappears from the cells, providing a near-isogenic model for well-controlled experiments (Aleman et al., 2003, Cancer Res. 63:3084-3091). FIG. 6, Panel C shows that the MDR1-expressing cells (MDR1-On) are two- to four-fold more sensitive than are MDR1-Off 14 cells, providing strong evidence that the increased sensitivity to NSC 73306 is mediated by MDR1 function. NSC 73306 does not block MDR1-mediated transport of other molecules, suggesting that it might avoid the well-documented side-effects observed in clinical trials of “classical” MDR1 inhibitors (Kellen, 2003, J. Exper. Ther. Oncol. 3:5-13).

To further identify compounds having an anti-proliferative effect that is potentiated by ABCB1, a larger set comprising 7500 DTP compounds is analyzed for positive correlations between antiproliferative activity and ABCB1 expression. The results of this analysis are presented in Table 9 below. It was assumed that any correlation with P>=0.35 A was significant.

TABLE 9
Compounds with an Antiproliferative Activity that is Positively Correlated
with ABCB1 Expression (From a Screen of 7500 Compounds)

| | | | |
|---|---|---|---|
| NSC 679285 | 0.350366317 | NSC 627025 | 0.351128441 |
| NSC 635543 | 0.351435667 | NSC 697131 | 0.352229413 |
| NSC 607301 | 0.352335924 | NSC 615537 | 0.352621346 |
| NSC 627452 | 0.353386557 | NSC 715729 | 0.354420669 |
| NSC 697132 | 0.355789371 | NSC 117028 | 0.357893409 |
| NSC 648072 | 0.357917331 | NSC 617959 | 0.358620454 |
| NSC 641288 | 0.36428927 | NSC 371168 | 0.364298669 |
| NSC 310618 | 0.369760098 | NSC 693931 | 0.370781734 |
| NSC 617966 | 0.37150536 | NSC 687141 | 0.37744196 |
| NSC 693326 | 0.389182931 | NSC 627451 | 0.389391105 |
| NSC 645542 | 0.392411887 | NSC 697130 | 0.392495416 |
| NSC 625349 | 0.393535291 | NSC 622927 | 0.400100248 |
| NSC 356777 | 0.40211398 | NSC 347512 | 0.410933115 |
| NSC 626670 | 0.417661071 | NSC 617961 | 0.422969914 |
| NSC 617278 | 0.423975493 | NSC 697135 | 0.42485689 |
| NSC 697137 | 0.429481982 | NSC 697678 | 0.4314303 |
| NSC 697128 | 0.434881741 | NSC 697120 | 0.443463864 |
| NSC 627450 | 0.445356374 | NSC 623069 | 0.458995086 |
| NSC 697124 | 0.463558577 | NSC 697129 | 0.466276635 |
| NSC 168468 | 0.483909859 | NSC 13875 | 0.485599049 |
| NSC 73306 | 0.511026556 | NSC 617963 | 0.531661338 |
| NSC 86715 | 0.532301975 | NSC 697125 | 0.535768232 |
| NSC 693871 | 0.681092945 | | |

Another set of compounds that have an antiproliferative activity that is potentiated by ABCB1 are listed in Table 10 below. These compounds are identified in a two step process: (1) a DTP set of 40,000 compounds was screened for compounds with structural homology to NSC 73306; and (2) identified homologous compounds were then assessed to determine whether they had an antiproliferative activity that positively correlates with ABCB1 expression.

TABLE 10
Compounds with an Antiproliferative Activity that is
Positively Correlated with ABCB1 Expression and that
have Structural Homology with NSC 73306)

| | | | |
|---|---|---|---|
| NSC117028 | NSC123053 | NSC142055 | NSC143095 |
| NSC168468 | NSC178123 | NSC2053 | NSC310618 |
| NSC32079 | NSC329287 | NSC33052 | NSC356778 |
| NSC382035 | NSC43321 | NSC50922 | NSC602313 |
| NSC605762 | NSC617934 | NSC621959 | NSC625893 |
| NSC627452 | NSC629730 | NSC629914 | NSC632731 |
| NSC634605 | NSC635534 | NSC636098 | NSC637446 |
| NSC638048 | NSC641613 | NSC642581 | NSC645257 |
| NSC645888 | NSC646285 | NSC647100 | NSC648062 |
| NSC649424 | NSC653148 | NSC655280 | NSC657576 |
| NSC657589 | NSC657924 | NSC658228 | NSC658339 |
| NSC658891 | NSC659488 | NSC665733 | NSC666715 |
| NSC666998 | NSC666999 | NSC667057 | NSC667925 |
| NSC668486 | NSC668493 | NSC668494 | NSC668495 |
| NSC668496 | NSC668497 | NSC668498 | NSC668499 |
| NSC669446 | NSC670960 | NSC671843 | NSC672001 |
| NSC672068 | NSC672073 | NSC672090 | NSC672099 |
| NSC673117 | NSC673454 | NSC675810 | NSC676911 |
| NSC676920 | NSC678372 | NSC679534 | NSC681112 |
| NSC681125 | NSC681602 | NSC682575 | NSC682714 |
| NSC682716 | NSC682719 | NSC683238 | NSC683505 |
| NSC685288 | NSC685459 | NSC688942 | NSC689530 |
| NSC691081 | NSC691215 | NSC691808 | NSC691980 |
| NSC692754 | NSC692756 | NSC692758 | NSC692759 |
| NSC693323 | NSC693325 | NSC693326 | NSC693335 |
| NSC693872 | NSC695592 | NSC697120 | NSC697124 |
| NSC697125 | NSC697129 | NSC697130 | NSC697132 |
| NSC697137 | NSC697678 | NSC697881 | NSC697933 |
| NSC698794 | NSC702616 | NSC702986 | NSC716764 |
| NSC716765 | NSC716766 | NSC716771 | NSC716772 |
| NSC7833 | | | |

One of the compounds listed in Table 10, NSC 168468, was tested in the MTT assay using the KB-3-1/KB-V1 cell pair. These tests confirmed that the NSC 168468 compound had an anti-proliferative activity that was potentiated by the ABCB1 transporter to an extent that was equivalent to or greater than the potentiation effect observed for NSC 73306. PSC 833 completely reversed sensitivity of KB-V1 cells to NSC 168468.

Two other homologs of NSC 73306, NSC 73304 and NSC 73305, are also tested in the assay system described in the above paragraphs. Similar to the results obtained with NSC 73306, assays on these other two compounds show that KB-V1 cells are several-fold more sensitive than the parental KB-3-1 and that PSC 833 completely reverses sensitivity of KB-V1 cells to NSC 73304 and NSC 73305.

All publications and patents mentioned in this specification are herein incorporated by reference to the same extent as if each individual publication or patent application was specifically and individually indicated to be incorporated by reference. While the invention has been described in connection with specific embodiments thereof it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-11. (canceled)

12: A method of inhibiting the growth of neoplastic cells in a subject comprising administering to the subject an antiproliferative agent, wherein the antiproliferative effect of the agent is potentiated by the ABCB1 transporter, wherein the antiproliferative agent is a compound of Structure Y or Structure Z: 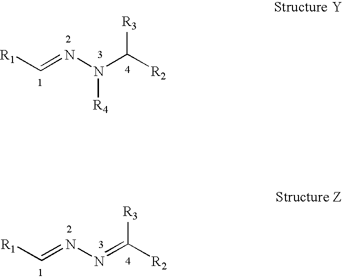
wherein R₁ may comprise one or two substituents on the carbon atom in position 1;
wherein each of R₁ are independently selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein when R₁ comprises two substituents on the carbon atom in position 1, the two substituents may cyclize to form a ring structure;
wherein each of R₁ may independently cyclize to form a ring structure;
wherein R₂ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein R₂ may cyclize to form a ring structure;
wherein R₃ comprises 0 or 1 substituents on the carbon atom at position 4;
wherein R₃ may be double bonded or single bonded to the carbon atom at position 4 of Structure Y or single bonded to the carbon atom at position 4 of Structure Z;
wherein R₃ is selected from the group consisting of a heteroatom, hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein R₃ may cyclize to form a ring structure;
wherein R₄ comprises 0 or 1 substituents on the nitrogen atom at position 3 of Structure Y or Structure Z;
wherein R₄ is selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group;
wherein R₄ may cyclize to form a ring structure.

13: A method according to claim 12 wherein R₂ is —N—R₅,
wherein R₂-may be single bonded or double bonded to the carbon atom at position of 4 of Structure Y or single bonded to the carbon atom at position 4 of Structure Z;
wherein R₅ comprises one or two substituents on the nitrogen atom;
wherein when R₅ comprises one substituent on the nitrogen atom and R₂ is single bonded to the carbon atom at position 4 of Structure Y or Z, R₅ may be double bonded to the nitrogen atom;
wherein each of R₅ may independently cyclize to form a ring structure;
wherein each of R₅ is independently selected from the group consisting of a hydrocarbon group, a substituted hydrocarbon group, a heterogeneous group, a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group.

14: The method of claim 1, wherein the antiproliferative agent is selected from NSC117028, NSC123053, NSC142055, NSC143095, NSC168468, NSC178123, NSC2053, NSC310618, NSC32079, NSC329287, NSC33052, NSC356778, NSC382035, NSC43321, NSC50922, NSC602313, NSC605762, NSC617934, NSC621959, NSC625893, NSC627452, NSC629730, NSC629914, NSC632731, NSC634605, NSC635534, NSC636098, NSC637446, NSC638048, NSC641613, NSC642581, NSC645257, NSC645888, NSC646285, NSC647100, NSC648062, NSC649424, NSC653148, NSC655280, NSC657576, NSC657589, NSC657924, NSC658228, NSC658339, NSC658891, NSC659488, NSC665733, NSC666715, NSC666998, NSC666999, NSC667057, NSC667925, NSC668486, NSC668493, NSC668494, NSC668495, NSC668496, NSC668497, NSC668498, NSC668499, NSC669446, NSC670960, NSC671843, NSC672001, NSC672068, NSC672073, NSC672090, NSC672099, NSC673117, NSC673454, NSC675810, NSC676911, NSC676920, NSC678372, NSC679534, NSC681112, NSC681125, NSC681602, NSC682575, NSC682714, NSC682716, NSC682719, NSC683238, NSC683505, NSC685288, NSC685459, NSC688942, NSC689530, NSC691081, NSC691215, NSC691808, NSC691980, NSC692754, NSC692756, NSC692758, NSC692759, NSC693323, NSC693325, NSC693326, NSC693335, NSC693872, NSC695592, NSC697120, NSC697124, NSC697125, NSC697129, NSC697130, NSC697933, NSC698794, NSC702616, NSC702986, NSC716764, NSC716765, NSC716766, NSC716771, NSC716772, NSC7833 or combinations thereof.

15: The method of claim 1, wherein the antiproliferative agent is selected from NSC 363997, NSC 359449, NSC 646946, NSC 363997, NSC 694268, NSC 634791, NSC 73304, NSC 73305, NSC 168468 or combinations thereof.

16: The method of claim 1, wherein the antiproliferative agent has the formula

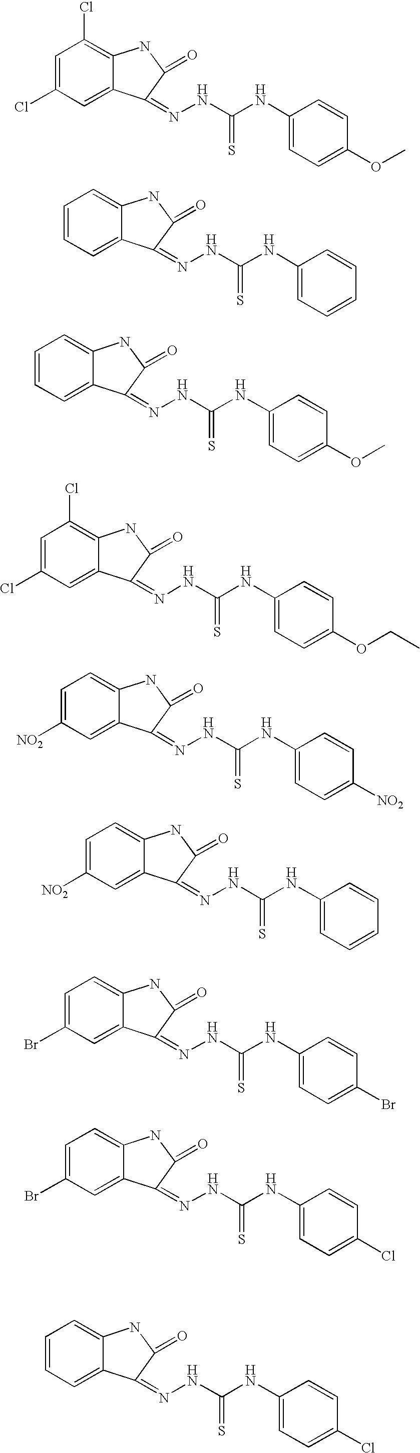

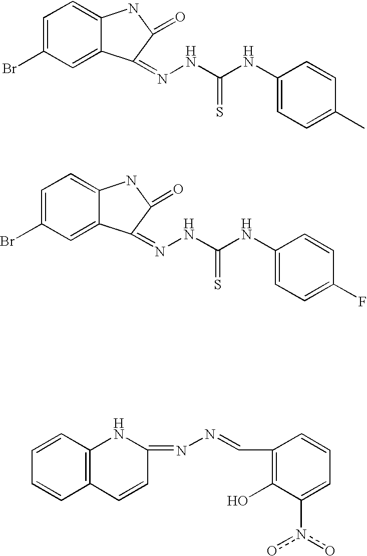

17: The method of claim 1, wherein the antiproliferative agent has the formula

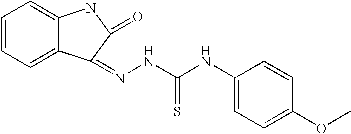

18: A method of inhibiting the growth of neoplastic cells in a subject comprising administering to a subject an antiproliferative agent, wherein the antiproliferative effect of the agent is potentiated by an ABCB1 transporter.

19: A method according to claim 1, wherein the neoplastic cells comprise a cancer in the subject and wherein the cancer exhibits a multidrug resistant phenotype.

20: A method according to claim 2, wherein the cancer exhibits a multidrug resistant phenotype at diagnosis.

21: A method according to claim 3, wherein the cancer is selected from the group consisting of colon carcinoma, renal carcinoma, hepatoma, adrenocortical carcinoma, and pancreatic carcinoma.

22: A method according to claim 2, wherein the subject has previously been treated with at least one anti-cancer therapeutic agent that is an ABCB1 substrate.

23: A method according to claim 5, wherein the anti-cancer therapeutic agent is selected from the group consisting of: a taxane, a vinca alkaloid, an anthracycline, and an epipodophyllotoxin.

24: A method according to claim 6 wherein the cancer is selected from the group consisting of breast cancer, ovarian cancer, sarcoma, small cell lung cancer, acute myeloid leukemia, chronic myeloid leukemia, acute lymphoblastic leukemia, non-Hodgkins lymphoma, B cell lymphoma, and T cell lymphoma.

25: A method of inhibiting the development of a multidrug resistance phenotype in a cancer in a subject comprising administering an antiproliferative agent to the subject, wherein the antiproliferative effect of the antiproliferative agent is potentiated by an ABCB1 transporter.

26: A method according to claim 8, wherein the antiproliferative agent is administered to the subject simultaneously with an anti-cancer therapeutic agent, wherein the anti-cancer therapeutic agent is an ABCB1 substrate.

27: A method of identifying therapeutic compounds having a therapeutic activity that is potentiated by the expression of an ABC gene comprising the steps of:
(a) determining the expression level of at least one ABC gene in a panel of cell lines;
(b) determining the level of therapeutic activity of at least one test compound on the panel of cell lines; and
(c) comparing the level of therapeutic activity with the expression level of the ABC gene, wherein a positive correlation between the level of therapeutic activity and the expression level of the ABC gene identifies the test compound as having an activity that is potentiated by the expression of an ABC gene.

28: A method of identifying therapeutic compounds as substrates for ABC transporters comprising the steps of:
(a) determining the expression level of at least one ABC gene in a panel of cell lines;
(b) determining the level of therapeutic activity of at least one test compound on the panel of cell lines; and
(c) comparing the level of therapeutic activity with the expression level of the ABC gene, wherein a negative correlation between the level of therapeutic activity and the expression level of the ABC gene identifies the test compound as a substrate of the ABC transporter encoded by an ABC gene.


SEQUENCE LISTING (94 sequences)

SEQ ID NO: 1 - DNA, 21 bp, Homo sapiens
gcactgaggaagatgctgaaa

SEQ ID NO: 2 - DNA, 23 bp, Homo sapiens
agttcctggaaggtcttgttcac

SEQ ID NO: 3 - DNA, 21 bp, Homo sapiens
catccccctggtgctgttctt

SEQ ID NO: 4 - DNA, 19 bp, Homo sapiens
gcttgggccgtgctattgg

SEQ ID NO: 5 - DNA, 23 bp, Homo sapiens
gccctctttacactcagttttca

SEQ ID NO: 6 - DNA, 23 bp, Homo sapiens
gacgagcagttgtcgtacctaat

SEQ ID NO: 7 - DNA, 23 bp, Homo sapiens
tggtcaaagcctgggaagaagta

SEQ ID NO: 8 - DNA, 23 bp, Homo sapiens
tccagggatacatgtcagggaat

SEQ ID NO: 9 - DNA, 23 bp, Homo sapiens
gggcccaatggtaggaggtagag

SEQ ID NO: 10 - DNA, 21 bp, Homo sapiens
tgaggaatgggcaagggaggt

SEQ ID NO: 11 - DNA, 19 bp, Homo sapiens
ccgtcaaggggctcaggaa

SEQ ID NO: 12 - DNA, 19 bp, Homo sapiens
gatggccacacggtcacac

SEQ ID NO: 13 - DNA, 23 bp, Homo sapiens
cccggccacgtgcgcatcaaaat

SEQ ID NO: 14 - DNA, 24 bp, Homo sapiens
ccaccgcgaaggctgccaagaaca

SEQ ID NO: 15 - DNA, 21 bp, Homo sapiens
agtgcgcgggctcttctttgt

SEQ ID NO: 16 - DNA, 24 bp, Homo sapiens
gttttccttcgcttttggctgata

SEQ ID NO: 17 - DNA, 23 bp, Homo sapiens
ccccatgatgaaagagcacagag

SEQ ID NO: 18 - DNA, 24 bp, Homo sapiens
aggatcccccaaaagacaataagg

SEQ ID NO: 19 - DNA, 24 bp, Homo sapiens
atggctcagatgatccctcctaca

SEQ ID NO: 20 - DNA, 24 bp, Homo sapiens
ctccgtttgaataagctccgtgaa

SEQ ID NO: 21 - DNA, 24 bp, Homo sapiens
tctcgccgaagtatatgggatgtt

SEQ ID NO: 22 - DNA, 21 bp, Homo sapiens
ggcttcggggagatgtgattg

SEQ ID NO: 23 - DNA, 25 bp, Homo sapiens
tgacatttattcaaagttaaaagca

SEQ ID NO: 24 - DNA, 25 bp, Homo sapiens
tagacactttatgcaaacatttcaa

SEQ ID NO: 25 - DNA, 21 bp, Homo sapiens
agggctggctggctgctttga

SEQ ID NO: 26 - DNA, 22 bp, Homo sapiens
acgtggcccatggtgttgttat

SEQ ID NO: 27 - DNA, 21 bp, Homo sapiens
acggctgagctcggataccac

SEQ ID NO: 28 - DNA, 18 bp, Homo sapiens
cctcggccccaaaactgc

SEQ ID NO: 29 - DNA, 21 bp, Homo sapiens
accgactgtctacggtccgaa

SEQ ID NO: 30 - DNA, 22 bp, Homo sapiens
tccatcggtttccacatcaagg

SEQ ID NO: 31 - DNA, 21 bp, Homo sapiens
tctggcccctcaaacctcacc

SEQ ID NO: 32 - DNA, 23 bp, Homo sapiens
tttcataccgccactgccaactc

SEQ ID NO: 33 - DNA, 21 bp, Homo sapiens
caaccgcaccaccatcgtagt

SEQ ID NO: 34 - DNA, 24 bp, Homo sapiens
aataagccagggaaaggagacaca

SEQ ID NO: 35 - DNA, 23 bp, Homo sapiens
tgggtcagggaaaagcacaatag

SEQ ID NO: 36 - DNA, 22 bp, Homo sapiens
ggggtccttcaaaatggctctt

SEQ ID NO: 37 - DNA, 19 bp, Homo sapiens
gggcccactgcattgtcgt

SEQ ID NO: 38 - DNA, 18 bp, Homo sapiens
cggccccggctttattgt

SEQ ID NO: 39 - DNA, 24 bp, Homo sapiens
gagggccgggtggactttgagaat

SEQ ID NO: 40 - DNA, 24 bp, Homo sapiens
cagtgggcaggccgtaggagatgt

SEQ ID NO: 41 - DNA, 24 bp, Homo sapiens
atgggcgatatctacggaaactga

SEQ ID NO: 42 - DNA, 21 bp, Homo sapiens
ggcgagctggataggcaaaat

SEQ ID NO: 43 - DNA, 24 bp, Homo sapiens
agggaaatcaagctcttaatgaag

SEQ ID NO: 44 - DNA, 25 bp, Homo sapiens
ataggtagacttatgatctacaaca

SEQ ID NO: 45 - DNA, 23 bp, Homo sapiens
agtggaacccctctctgtttaag

SEQ ID NO: 46 - DNA, 23 bp, Homo sapiens
cctgatacgtcttggtcttcatc

SEQ ID NO: 47 - DNA, 22 bp, Homo sapiens
tccttgcgcagctggattacat

SEQ ID NO: 48 - DNA, 22 bp, Homo sapiens
tcgctgaagtgagagtagattg

SEQ ID NO: 49 - DNA, 20 bp, Homo sapiens
cagagaaggtgcaggtgaca

SEQ ID NO: 50 - DNA, 20 bp, Homo sapiens
ctaaagcagcatagacgccc

SEQ ID NO: 51 - DNA, 20 bp, Homo sapiens
tgatgagccgtatgttttgc

SEQ ID NO: 52 - DNA, 20 bp, Homo sapiens
cttcggaacggacttgacat

SEQ ID NO: 53 - DNA, 24 bp, Homo sapiens
aggggcaagaaagagaaggtgagg

SEQ ID NO: 54 - DNA, 24 bp, Homo sapiens
gagggggtcgtccaggatgtagat

SEQ ID NO: 55 - DNA, 18 bp, Homo sapiens
ggcccgggcatccaggtt

SEQ ID NO: 56 - DNA, 24 bp, Homo sapiens
tttcatctacgcgagcattgttct

SEQ ID NO: 57 - DNA, 22 bp, Homo sapiens
catttttggccttcatcacatt

SEQ ID NO: 58 - DNA, 21 bp, Homo sapiens
tgccttccgagtcagtttcag

SEQ ID NO: 59 - DNA, 24 bp, Homo sapiens
ctgctaaaccggatcatcctagcc

SEQ ID NO: 60 - DNA, 24 bp, Homo sapiens
cgaggaacacaggtgtgacatagg

SEQ ID NO: 61 - DNA, 20 bp, Homo sapiens
gctacaaagttggcagaggc

SEQ ID NO: 62 - DNA, 24 bp, Homo sapiens
tcccaggcatacaattttagaagt

SEQ ID NO: 63 - DNA, 23 bp, Homo sapiens
ggctccggcaagtcttccctgtt

SEQ ID NO: 64 - DNA, 23 bp, Homo sapiens
agatagctccggcccccttcacc

SEQ ID NO: 65 - DNA, 20 bp, Homo sapiens
ccacggccctgcacaacaag

SEQ ID NO: 66 - DNA, 23 bp, Homo sapiens
ggaattgccaaaagccacgaaca

SEQ ID NO: 67 - DNA, 19 bp, Homo sapiens
caccgcctctatggactcc

SEQ ID NO: 68 - DNA, 20 bp, Homo sapiens
tcaatctcaggcactggggt

SEQ ID NO: 69 - DNA, 24 bp, Homo sapiens
accaggtgatctacccggactcag

SEQ ID NO: 70 - DNA, 24 bp, Homo sapiens
ctcacggcgctggtgcattcatcc

SEQ ID NO: 71 - DNA, 20 bp, Homo sapiens
tggcctgattcgacctctcc

SEQ ID NO: 72 - DNA, 23 bp, Homo sapiens
gtctgcagcgtttctcttccact

SEQ ID NO: 73 - DNA, 20 bp, Homo sapiens
ctcggcctgcacggtaagaa

SEQ ID NO: 74 - DNA, 24 bp, Homo sapiens
tggcagcgatgaagttgagtaagt

SEQ ID NO: 75 - DNA, 25 bp, Homo sapiens
ggatctgagcctaaagatctccgag

SEQ ID NO: 76 - DNA, 23 bp, Homo sapiens
gggtcccgtcagtgaagaatggc

SEQ ID NO: 77 - DNA, 21 bp, Homo sapiens
ggttgcctatccctcgtccag

SEQ ID NO: 78 - DNA, 21 bp, Homo sapiens
tgtcccctttgccagccttag

SEQ ID NO: 79 - DNA, 24 bp, Homo sapiens
acaggctggggaagaagagaaagt

SEQ ID NO: 80 - DNA, 23 bp, Homo sapiens
cagggctgcaaaaacattaccac

SEQ ID NO: 81 - DNA, 24 bp, Homo sapiens
tagggcgttaccatcagcatttac

SEQ ID NO: 82 - DNA, 23 bp, Homo sapiens
gaccagcatcataccaccctcaa

SEQ ID NO: 83 - DNA, 20 bp, Homo sapiens
ggggcatcagacacgctcac

SEQ ID NO: 84 - DNA, 21 bp, Homo sapiens
gttggggcagggcatagtcat

SEQ ID NO: 85 - DNA, 21 bp, Homo sapiens
caggaagattagacactgtgg

SEQ ID NO: 86 - DNA, 21 bp, Homo sapiens
gaaaggggaatggagagaaga

SEQ ID NO: 87 - DNA, 22 bp, Homo sapiens
ccgcgacagtttccaatgacct

SEQ ID NO: 88 - DNA, 24 bp, Homo sapiens
gccgaagagctgctgagaactgta

SEQ ID NO: 89 - DNA, 24 bp, Homo sapiens
ggtctggatagcgcctcttgtttc

SEQ ID NO: 90 - DNA, 23 bp, Homo sapiens
atggggcagggacctcgttcttc

SEQ ID NO: 91 - DNA, 23 bp, Homo sapiens
gccgactgtgcatgactgctctg

SEQ ID NO: 92 - DNA, 22 bp, Homo sapiens
ttacattcttgggtccgctcag

SEQ ID NO: 93 - DNA, 22 bp, Homo sapiens
ccgggggcttcatgataaactt

SEQ ID NO: 94 - DNA, 23 bp, Homo sapiens
ctgaggccaatgacgatgaggta